(19) United States
(12) Patent Application Publication
(10) Pub. No.: US 2024/0076704 A1
(43) Pub. Date: Mar. 7, 2024

(54) PRODUCTION OF BIOPRODUCT IN A HOST CELL

(71) Applicant: INBIOSE N.V., Zwijnaarde (BE)

(72) Inventors: Joeri Beauprez, Zwijnaarde (BE); Pieter Coussement, Zwijnaarde (BE); Nausicaä Lannoo, Zwijnaarde (BE); Gert Peters, Zwijnaarde (BE); Kristof Vandewalle, Zwijnaarde (BE); Annelies Vercauteren, Zwijnaarde (BE); Sofie Aesaert, Zwijnaarde (BE); Thomas Decoene, Zwijnaarde (BE)

(21) Appl. No.: 17/767,400

(22) PCT Filed: Oct. 14, 2020
(86) PCT No.: PCT/EP2020/078830

§ 371 Date: Apr. 7, 2022

(30) Foreign Application Priority Data
Oct. 14, 2019 (EP) 19202978.3

(51) Int. Cl. C12P 19/02; C07K 14/245; C12N 1/20; C12N 15/70; C12P 19/04; C12P 19/12; C12P 19/44

(52) U.S. Cl. CPC C12P 19/02; C07K 14/245; C12N 1/20; C12N 15/70; C12P 19/04; C12P 19/12; C12P 19/44; C12R 2001/19

(57) ABSTRACT

Described is a method of producing bioproducts by fermentation with a genetically modified cell, as well as to the genetically modified cell used in the method. The cell is genetically modified to produce a bioproduct and is further genetically modified by reducing the expression of at least one endogenous membrane protein encoding gene and/or mutating the expression of the endogenous membrane protein.

### CROSS-REFERENCE TO RELATED APPLICATIONS

This application is a national phase entry under 35 U.S.C. § 371 of International Patent Application PCT/EP2020/078830, filed Oct. 14, 2020, designating the United States of America and published as International Patent Publication WO 2021/074182 A1 on Apr. 22, 2021, which claims the benefit under Article 8 of the Patent Cooperation Treaty to European Union Patent Application Serial No. 19202978.3, filed Oct. 14, 2019.

### TECHNICAL FIELD

The present disclosure is in the technical field of synthetic biology and metabolic engineering. More particularly, the disclosure is in the technical field of fermentation of metabolically engineered host cells. The disclosure describes a method of producing bioproducts by fermentation with a genetically modified cell, as well as to the genetically modified cell used in the method. The cell is genetically modified to produce a bioproduct and is further genetically modified by reducing the expression of at least one endogenous membrane protein encoding gene and/or mutating the expression of the endogenous membrane protein.

### BACKGROUND

Industrial production of bioproducts can be performed by fermentation of cells enabled to produce the bioproducts. The preparation of cultures is labor intensive, occupying much space and equipment, and there is a considerable risk of contamination with spoilage bacteria and/or phages during the step of propagation. The failure of bacterial cultures by bacteriophage (phage) infection and multiplication is a major problem with the industrial use of bacterial cultures. There are many different types of phages with varying mechanisms to attack bacteria. Moreover, new strains of bacteriophages appear.

Strategies used in industry to minimize bacteriophage infection, and thus failure of a bacterial culture, include the use of: (i) mixed starter cultures; and (ii) the alternate use of strains having different phage susceptibility profiles (strain rotation).

The complex composition of mixed starter cultures ensures that a certain level of resistance to phage attack is present. However, repeated sub-culturing of mixed strain cultures leads to unpredictable changes in the distribution of individual strains and eventually undesired strain dominance. This in turn may lead to increased susceptibility to phage attack and risk of fermentation failures.

Rotation of selected bacterial strains that are sensitive to different phages is another approach to limit phage development. However, it is difficult and cumbersome to identify and select a sufficient number of strains having different phage type profiles to provide an efficient and reliable rotation program. In addition, the continuous use of strains requires careful monitoring for new infectious phages and the need to quickly substitute a strain that is infected by the new bacteriophage by a resistant strain, in manufacturing plants where large quantities of bulk starter cultures are made ahead of time, such a quick response is usually not possible.

There is a continuing need in the art to provide improved bacterial strains for use in fermentative production industry—such as bacterial strains producing a desired product. Improved bacterial strains that are phage resistant are particularly desirable.

The evolutionary pressure imposed by phage predation on bacteria has led to the development of efficient resistance systems to protect bacteria from phage infection. Makarova et al. (J Bacteriol. 2011, November, 193(21): 6039-6056) disclosed that a substantial fraction of bacterial and archaeal genomes is dedicated to phage defense and that the defense genes are typically clustered in genomic islands termed defense islands. Some of these systems include restriction-modification systems, abortive infection mechanisms, CRISPR/Cas adaptive defense system, prokaryotic argonaute system, BREX system (see WO2015/059690) and DISARM system (WO2018/142416). Others found that for specific bacterial strains specific deletion of a membrane protein encoding gene makes the cell more resistant to phage attack (e.g., fhuA gene as disclosed in V. Braun (2009) J Bacteriol. 191(11):3431-3436 and Link et al., 1997, J. Bact. 179: 6228-8237).

However, in the fermentative production of specific bioproducts, more in particular monosaccharide, phosphorylated monosaccharide, activated monosaccharide, disaccharide, oligosaccharide or glycolipid, hesitation exists for engineering other genetic loci than strictly needed for the production of the bioproduct as such further engineering steps might have a negative effect on growth of the cell and/or production of the desired bioproduct. Furthermore, full gene deletions may hamper normal cell function, depending on the importance of the role of the membrane protein.

### BRIEF SUMMARY

Described is a novel phage resistance system, which is to be used in E. coli producing bioproducts such as monosaccharide, phosphorylated monosaccharide, activated monosaccharide, disaccharide, oligosaccharide or glycolipid. The phage resistance system comprises a reduced expression of at least one endogenous membrane protein encoding gene and/or a mutation of the endogenous membrane protein encoding gene and more preferably a reduced expression and/or mutation of an endogenous outer membrane protein encoding gene. The newly discovered system effectively and efficiently protects against phages and at the same time is not negatively influencing the bioproduct productivity and/or growth of the fermenting E. coli bacteria.

Specifically, the phage resistance system confers complete or partial resistance against E. coli phages spanning a wide phylogeny of phage types, including lytic and temperate (also referred lysogenic) phages, even in the first cycle of infection.

This disclosure also provides methods for enhanced production of at least one desired bioproduct. The bioproduct is obtained with a genetically modified host cell comprising the phage resistance system of the disclosure.

The words used in this specification to describe this disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of this disclosure disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of this disclosure, and although specific terms are employed, the terms are used in a descriptive sense only and not for purposes of limitation, the scope of this disclosure being set forth in the following claims. It must be understood that the illustrated embodiments have been set forth only for the purposes of example and that it should not be taken as limiting this disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

According to the disclosure, the term “membrane protein” refers to a protein found in biological membranes or cell envelope and commonly known by a person skilled in the art (Lodish H, Berk A, Zipursky S L, et al., 2000 and Silhavy et al. 2010). It is the protein component of the cytoplasmic membrane, the outer membrane or the cell wall. Membrane proteins may be integral, peripheral or lipid anchored proteins or combinations there off. The term refers to proteins that are part of or interact with the cell membrane and can control, for instance, the flow of molecules, information across the cell or form a structural part of the membrane. The membrane proteins are preferably involved in transport, be it import into or export out of the cell.

The term “membrane protein encoding gene(s)” as used herein encompasses polynucleotides that include a sequence encoding a membrane protein of this disclosure. The term also encompasses polynucleotides that include a single continuous region or discontinuous regions encoding the membrane protein (for example, interrupted by integrated phage or an insertion sequence or editing) together with additional regions that also may contain coding and/or non-coding sequences.

According to the disclosure, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s),” according to the disclosure. Moreover, DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

The term “polynucleotide encoding a polypeptide” as used herein encompasses polynucleotides that include a sequence encoding a polypeptide of this disclosure. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized” or “synthetic,” as the term is used herein, means any synthetically generated sequence and not directly isolated from a natural source.

The term “recombinant” or “transgenic” or “genetically modified,” as used herein with reference to a cell or host cell indicates that the bacterial cell replicates a heterologous nucleic acid, or expresses a peptide or protein encoded by a heterologous nucleic acid (i.e., a sequence “foreign to the cell” or a sequence “foreign to the location or environment in the cell”). Such cells are described to be transformed with at least one heterologous or exogenous gene, or are described to be transformed by the introduction of at least one heterologous or exogenous gene. Recombinant or transgenic cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified or its expression has been modified without removing the nucleic acid from the cell; such modifications include those obtained by gene replacement, replacement of a promoter; site-specific mutation; and related techniques. Accordingly, a “recombinant polypeptide” is one that has been produced by a recombinant cell. A “heterologous sequence” or a “heterologous nucleic acid,” as used herein, is one that originates from a source foreign to the particular cell (e.g., from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g., by transfection, transformation, conjugation or transduction, into the genome of the host microorganism cell, wherein techniques may be applied that will depend on the host cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989).

The term “endogenous,” within the context of the disclosure refers to any polynucleotide, polypeptide or protein sequence that is a natural part of a cell and is occurring at its natural location in the cell chromosome.

The term “heterologous” or “exogenous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “modified expression” of a gene relates to a change in expression compared to the wild type expression of the gene in any phase of the production process of the encoded protein. The modified expression is either a lower or higher expression compared to the wild type, wherein the term “higher expression” is also defined as “overexpression” of the gene in the case of an endogenous gene or “expression” in the case of a heterologous gene that is not present in the wild type strain. Lower expression or reduced expression is obtained by way of common well-known technologies for a skilled person (such as the usage of siRNA, CrispR, CrispRi, recombineering, homologous recombination, ssDNA mutagenesis, RNAi, miRNA, asRNA, mutating genes, knocking-out genes, transposon mutagenesis, . . . ), which are used to change the genes in such a way that they are less-able (i.e., statistically significantly ‘less-able’ compared to a functional wild-type gene) or completely unable (such as knocked-out genes) to produce functional final products. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can be obtained, for instance, by mutating one or more base pairs in the promoter sequence or changing the promoter sequence fully to a constitutive promoter with a lower expression strength compared to the wild type or an inducible promoter that result in regulated expression or a repressible promoter that results in regulated expression. Overexpression or expression is obtained by way of common well-known technologies for a skilled person, wherein the gene is part of an “expression cassette” that relates to any sequence in which a promoter sequence, untranslated region sequence (containing either a ribosome binding sequence or Shine Dalgarno sequence), a coding sequence (for instance, a membrane protein gene sequence) and optionally a transcription terminator is present, and leading to the expression of a functional active protein. The expression is either constitutive or conditional or regulated.

The term “constitutive expression” is defined as expression that is not regulated by transcription factors other than the subunits of RNA polymerase (e.g., the bacterial sigma factors) under certain growth conditions. Non-limiting examples of such transcription factors are CRP, LacI, ArcA, Cra, and IclR in E. coli. These transcription factors bind on a specific sequence and may block or enhance expression in certain growth conditions. RNA polymerase binds a specific sequence to initiate transcription, for instance, via a sigma factor in prokaryotic hosts.

The term “regulated expression” is defined as expression that is regulated by transcription factors other than the subunits of RNA polymerase (e.g., bacterial sigma factors) under certain growth conditions. Examples of such transcription factors are described above. Commonly expression regulation is obtained by way of an inducer or repressor, such as but not limited to IPTG, arabinose, rhamnose, fucose, allo-lactose or pH shifts, or temperature shifts or carbon depletion or substrates or the produced product or chemical repression.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

The term “wild type” refers to the commonly known genetic or phenotypical situation as it occurs in nature.

“Variant(s)” as the term is used herein, is a polynucleotide or polypeptide that differs from a reference polynucleotide or polypeptide, respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the disclosure contemplates making functional variants by modifying the structure of a membrane protein as used in the disclosure. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, and in case of the disclosure to provide better yield, productivity, and/or growth speed than a cell without the variant.

The term “functional homolog” as used herein describes those molecules that have sequence similarity and also share at least one functional characteristic such as a biochemical activity. Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. A functional homolog and the reference polypeptide may be naturally occurring polypeptides, and the sequence similarity may be due to convergent or divergent evolutionary events. Functional homologs are sometimes referred to as orthologs, where “ortholog,” refers to a homologous gene or protein that is the functional equivalent of the referenced gene or protein in another species. Functional homologs can be identified by analysis of nucleotide and polypeptide sequence alignments. For example, performing a query on a database of nucleotide or polypeptide sequences can identify homologs of interesting polypeptides. Sequence analysis can involve BLAST, Reciprocal BLAST, or PSI-BLAST analysis of non-redundant databases using amino acid sequence of an interesting polypeptide as the reference sequence. Amino acid sequence is, in some instances, deduced from the nucleotide sequence. Typically, those polypeptides in the database that have greater than 40 percent sequence identity are candidates for further evaluation for suitability as an interesting polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in interesting polypeptides, e.g., conserved functional domains.

“Fragment,” with respect to a polynucleotide, refers to a clone or any part of a polynucleotide molecule, particularly a part of a polynucleotide that retains a usable, functional characteristic or in other cases the fragment is non-functional. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about 9 consecutive nucleotides, for example at least about 30 nucleotides or at least about 50 nucleotides of any of the sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide.

Fragments may additionally or alternatively include subsequences of polypeptides and protein molecules, or a subsequence of the polypeptide. In some cases, the fragment or domain is a subsequence of the polypeptide that performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example, at least about 20 amino acid residues in length, for example, at least about 30 amino acid residues in length. Preferentially a fragment is a functional fragment that has at least one property or activity of the polypeptide from which it is derived, such as, for example, the fragment can include a functional domain or conserved domain of a polypeptide. A domain can be characterized, for example, by a Pfam or Conserved Domain Database (CDD) designation. According to the disclosure, such functional fragment has a reduced and/or abolished bacteriophage binding capacity but still performs another of its properties or activities as the original, full length polypeptide.

The term “bioproduct” as used herein refers to the group of molecules comprising at least one monosaccharide as defined herein. More in particular, the term bioproduct is chosen from the list comprising, preferably including, monosaccharide, phosphorylated monosaccharide, activated monosaccharide, disaccharide, oligosaccharide and glycolipid.

The term “monosaccharide” as used herein refers to saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, Sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O—[(R)-1-carboxyethyl]-2-deoxy-D-glucopyranose, 2-Acetamido-3-O—[(R)-carboxyethyl]-2-deoxy-D-glucopyranose, 2-Glycolylamido-3-O—[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, glucose, galactose, N-acetylglucosamine, glucosamine, mannose, xylose, N-acetylmannosamine, N-acetylneuraminic acid, N-glycolylneuraminic acid, a sialic acid, N-acetylgalactosamine, galactosamine, fucose, rhamnose, glucuronic acid, gluconic acid, fructose and polyols.

The term “phosphorylated monosaccharide” as used herein refers to one of the above listed monosaccharides that is phosphorylated. Examples of phosphorylated monosaccharides include but are not limited to glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these phosphorylated monosaccharides are precursors or intermediates for the production of activated monosaccharide.

The term “activated monosaccharide” as used herein refers to activated forms of monosaccharides, such as the monosaccharides as listed here above. Examples of activated monosaccharides include but are not limited to GDP-fucose, GDP-mannose, CMP-N-acetylneuraminic acid, CMP-N-glycolylneuraminic acid, UDP-glucuronate, UDP-N-acetylgalactosamine, UDP-glucose, UDP-galactose, CMP-sialic acid; and UDP-N-acetylglucosamine. Activated monosaccharides, also known as nucleotide sugars, act as glycosyl donors in glycosylation reactions. Those reactions are catalyzed by a group of enzymes called glycosyltransferases.

The term “disaccharide” as used herein refers to a saccharide polymer containing two simple sugars, i.e., monosaccharides. Such disaccharides contain monosaccharides as described above and are preferably selected from the list of monosaccharides as used herein above. Examples of disaccharides comprise lactose, N-acetyllactosamine, and Lacto-N-biose.

“Oligosaccharide,” as the term is used herein, and as generally understood in the state of the art, refers to a saccharide polymer containing a small number, typically three to fifteen, of simple sugars, i.e., monosaccharides. Preferably, the oligosaccharide as described herein contains monosaccharides selected from the list as used herein above. Examples of oligosaccharides include but are not limited to Lewis-type antigen oligosaccharides, mammalian milk oligosaccharides and human milk oligosaccharides.

As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialylactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

As used herein, the term “Lewis-type antigens” comprise the following oligosaccharides: H1 antigen, which is Fucα1-2Galβ1-3GlcNAc, or in short 2′FLNB; Lewis^{a}, which is the trisaccharide Galβ1-3[Fucα1-4]GlcNAc, or in short 4-FLNB; Lewis^{b}, which is the tetrasaccharide Fucα1-2Galβ1-3[Fucα1-4]GlcNAc, or in short DiF-LNB; sialyl Lewis^{a}, which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H2 antigen, which is Fucα1-2Galβ1-4GlcNAc, or otherwise stated 2′fucosyl-N-acetyl-lactosamine, in short 2′FLacNAc; Lewis^{x}, which is the trisaccharide Galβ1-4[Fucα1-3]GlcNAc, or otherwise known as 3-Fucosyl-N-acetyl-lactosamine, in short 3-FLacNAc, Lewis^{y}, which is the tetrasaccharide Fucα1-2Galβ1-4[Fucα1-3]GlcNAc and sialyl Lewis^{x}, which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GlcNAc.

As used herein, a ‘sialylated oligosaccharide’ is to be understood as a charged sialic acid containing oligosaccharide, i.e., an oligosaccharide having a sialic acid residue. It has an acidic nature. Some examples are 3-SL (3′-sialyllactose), 3′-sialyllactosamine, 6-SL (6′-sialyllactose), 6′-sialyllactosamine, oligosaccharides comprising 6′-sialyllactose, SGG hexasaccharide (Neu5Acα-2,3Galβ-1,3GalNacβ-1,3Galα-1,4Galβ-1,4Gal), sialylated tetrasaccharide (Neu5Acα-2,3Galβ-1,4GlcNacβ-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue or residues, including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GD1a Neu5Acα-2,3Galβ-1,3GalNAcβ-1,4(Neu5Acα-2,3)Galβ-1,4Glc, GT1a Neu5Acα-2,8Neu5Acα-2,3Galβ-1,3GalNAcβ-1,4(Neu5Acα-2,3)Galβ-1,4Glc, GD2 GalNAcβ-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAc β-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAc β-1,4(Neu5Acα-2,8Neu5Acα-2,8Neu5Acα2,3)Galβ-1,4Glc, GP1c Neu5Acα-2,8Neu5Acα-2,3Galβ-1,3GalNAc β-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fuca-1,2Galβ-1,3GalNAcβ-1,4(Neu5Acα-2,3)Gal β-1,4Glc; all of which may be extended to the production of the corresponding gangliosides by reacting the above oligosaccharide moieties with ceramide or synthetizing the above oligosaccharides on a ceramide.

A ‘fucosylated oligosaccharide’ as used herein and as generally understood in the state of the art is an oligosaccharide that is carrying a fucose-residue. Examples comprise 2′-fucosyllactose (2′FL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNF I), Lacto-N-fucopentaose II (LNF II), Lacto-N-fucopentaose III (LNF III), lacto-N-fucopentaose V (LNF V), lacto-N-fucopentaose VI (LNF VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose.

A ‘neutral oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2′, 3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

A ‘fucosylation pathway’ as used herein is a biochemical pathway comprising the enzymes and their respective genes, mannose-6-phosphate isomerase, phosphomannomutase, mannose-1-phosphate guanylyltransferase, GDP-mannose 4,6-dehydratase, GDP-L-fucose synthase and/or the salvage pathway L-fucokinase/GDP-fucose pyrophosphorylase, combined with a fucosyltransferase leading to α1,2; α1,3 α1,4 or α1,6 fucosylated oligosaccharides or fucosylated oligosaccharide containing bioproduct.

A ‘sialylation pathway’ is a biochemical pathway comprising the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, Glucosamine 6-phosphate N-acetyltransferase, N-AcetylGlucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, and/or CMP-sialic acid synthase, combined with a sialyltransferase leading to α2,3; α2,6 α2,8 sialylated oligosaccharides or sialylated oligosaccharide containing bioproduct.

A ‘galactosylation pathway’ as used herein is a biochemical pathway comprising the enzymes and their respective genes, galactose-1-epimerase, galactokinase, glucokinase, galactose-1-phosphate uridylyltransferase, UDP-glucose 4-epimerase, glucose-1-phosphate uridylyltransferase, and/or glucophosphomutase, combined with a galactosyltransferase leading to an alpha or beta bound galactose on the 2, 3, 4, 6 hydroxyl group of a mono, di, oligo or polysaccharide containing bioproduct.

An ‘N-acetylglucosamine carbohydrate pathway’ as used herein is a biochemical pathway comprising the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridylyltransferase, glucosamine-1-phosphate acetyltransferase, and/or glucosamine-1-phosphate acetyltransferase, combined with a glycosyltransferase leading to an alpha or beta bound N-acetylglucosamine on the 3, 4, 6 hydroxylgroup of a mono, di, oligo or polysaccharide containing bioproduct.

As used herein, the term “glycolipid” refers to any of the glycolipids that are generally known in the art. Glycolipids (GLs) can be subclassified into Simple (SGLs) and Complex (CGLs) glycolipids. Simple GLs, sometimes called saccharolipids, are two-component (glycosyl and lipid moieties) GLs in which the glycosyl and lipid moieties are directly linked to each other. Examples of SGLs include glycosylated fatty acids, fatty alcohols, carotenoids, hopanoids, sterols or paraconic acids. Bacterially produced SGLs can be classified into rhamnolipids, glucolipids, trehalolipids, other glycosylated (non-trehalose containing) mycolates, trehalose-containing oligosaccharide lipids, glycosylated fatty alcohols, glycosylated macro-lactones and macro-lactams, glycomacrodiolides (glycosylated macrocyclic dilactones), glyco-carotenoids and glyco-terpenoids, and glycosylated hopanoids/sterols. Complex glycolipids (CGLs) are, however, structurally more heterogeneous, as they contain, in addition to the glycosyl and lipid moieties, other residues like, for example, glycerol (glycoglycerolipids), peptide (glycopeptidolipids), acylated-sphingosine (glycosphingolipids), or other residues (lipopolysaccharides, phenolic glycolipids, nucleoside lipids).

The term “phage insensitive” or “phage resistant” or “phage resistance” or “phage resistant profile” is understood to mean a bacterial strain that is less sensitive, and preferably insensitive to infection and/or killing by phage and/or growth inhibition.

As used herein, the terms “anti-phage activity” or “resistant to infection by at least one phage” refers to an increase in resistance of a bacterial cell expressing a functional phage resistance system to infection by at least one phage family in comparison to a bacterial cell of the same species under the same developmental stage (e.g., culture state) that does not express a functional phage resistance system, as may be determined by e.g., bacterial viability, phage lysogeny, phage genomic replication and phage genomic degradation. The phage can be a lytic phage or a temperate (lysogenic) phage as further described hereinbelow. According to specific embodiments, the cell is 100% resistant as described above.

According to other specific embodiments, the increase is by at least 5%, by at least a 10%, at least 20%, at least 30%, at least 40%, at least 50%, at least 60%, at least 70%, at least 80%, at least 90% or more than 99% as compared to a fully phage resistant cell.

Assays for testing phage resistance are well known in the art and described hereinbelow.

As used herein, “abortive infection (Abi)” refers to a controlled cell death of an infected bacterial cell that takes place prior to the production of phage progeny, thus protecting the culture from phage propagation. Methods of analyzing Abi include, but are not limited to cell survival assays using high multiplicity of infection, one step growth assays and determination of phage DNA replication by e.g., DNA sequencing and southern blot analysis as further described hereinbelow.

As used herein, “adsorption” refers to the attachment to the host (e.g., bacteria) cell surface via plasma membrane proteins and glycoproteins. Methods of analyzing phage adsorption include, but are not limited to enumerating free phages in bacterial cultures infected with the phages immediately after phage addition and at early time points (e.g., 30 minutes) following phage addition as further described hereinbelow.

As used herein, the term “prevent” or “preventing” refers to a decrease in activity (e.g., phage genomic replication, phage lysogeny) in bacteria expressing a functional phage resistance system in comparison to bacteria of the same species under the same developmental stage (e.g., culture state) that does not express a functional phage resistance system. According to specific embodiments, the decrease is at least 1.5 fold, at least 2 fold, at least 3 fold, at least 5 fold, at least 10 fold, or at least 20 fold as compared to same in the absence of the functional phage resistance system. According to other specific embodiments, the decrease is by at least 5%, by at least a 10%, at least 20%, at least 30%, at least 40%, at least 50%, at least 60%, at least 70%, at least 80%, at least 90%, at least 95% or 99% or 100% as compared to same in the absence of the functional phage resistance system.

As used herein, “phage genomic replication” refers to production of new copies of the phage genome that can be dsDNA or ssDNA. Methods of analyzing phage genomic replication are well known in the art and described e.g., in Goldfarb et al., EMBO J, 34, 169-183.

As used herein, the term “lysogeny” refers to the incorporation of the phage genetic material inside the genome of the host (e.g., bacteria). Methods of analyzing phage lysogeny are well known in the art and include, but not limited to, DNA sequencing and PCR analysis. Typically, when a temperate phage infects a bacterium, its genetic material becomes circular before it incorporates into the bacterial genome. Circularization of phage genome can be analyzed by methods well known in the art including, but not limited to, PCR analysis as described in the art. When referring to “degradation of phage genome” the meaning is the cleavage of the foreign phage genome by the host bacteria. Method of analyzing genomic degradation are well known in the art including, but not limited to, DNA sequencing and PCR analysis.

As used herein, the phrase “reducing and/or abolishing the bacteriophage binding capacity” refers to a reduced or decreased ability of the membrane protein to bind bacteriophage, such decrease is by at least 5%, by at least 10%, at least 20%, at least 30%, at least 40%, at least 50%, at least 60%, at least 70%, at least 80%, at least 90%, at least 95%, at least 96%, at least 97%, at least 98%, at least 99% or 100% as compared to same in the absence of the functional phage resistance system.

The term “non-native” as used herein with reference to a bioproduct indicates that the bioproduct is i) not naturally produced or ii) when naturally produced not in the same amounts by the cell; and that the cell has been genetically modified to be able to produce the bioproduct or have a higher production of the bioproduct.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, glycolipids, proteins or nucleic acids of this disclosure are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For example, for oligosaccharides, e.g., 3-fucosyllactose, purity can be determined using methods such as but not limited to thin layer chromatography, gas chromatography, NMR, HPLC, capillary electrophoresis or mass spectroscopy.

The terms “identical” or “percent identity” or “% identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity can be determined using BLAST and PSI-BLAST (Altschul et al., 1990, J Mol Biol 215:3, 403-410; Altschul et al., 1997, Nucleic Acids Res 25: 17, 3389-402). For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

As used herein, the term “cell productivity index (CPI)” refers to the mass of the product produced by the recombinant cells divided by the mass of the recombinant cells produced in the culture.

As used herein, the term “normalized production” or “normalized productivity” refers to the mass of the product produced by the recombinant cells divided by the mass of the recombinant cells produced in the culture (CPI), and further normalized to a particular reference value (which is unless otherwise stated the averaged CPI value of a reference strain in the same experiment).

### BRIEF DESCRIPTION OF THE DRAWINGS

The following drawings and examples will serve as further illustration and clarification of the disclosure and are not intended to be limiting.

FIG. 1 shows the normalized absorbance measured at 600 nm after 72 hours of cultivation of a 2′FL and DiFL production strain with the wild type tolC gene or the tolC_IS1 or tolC_2 mutation.

FIG. 2 shows the normalized production of 2′FL and DiFL after 72 hours of cultivation of a 2′FL and DiFL production strain with the wild type tolC gene or the tolC_IS1 or tolC_2 mutation.

FIG. 3 shows the normalized growth speed of a 2′FL and DiFL production strain with the wild type tolC gene or the tolC_IS1 or tolC_2 mutation.

FIG. 4 shows the normalized production of 2′FL or 3FL after 72 hours of cultivation by strains with the wild type tolC or the tolC_2 mutation.

FIG. 5 shows the normalized production of LNnT after 72 hours of cultivation of a strain with the wild type fhuA gene (=reference strain, Ref) or the fhuA-fs mutation.

FIG. 6 shows the normalized growth speed of both strains after 72 hours of cultivation of a strain with the wild type fhuA gene (=reference strain, Ref) or the fhuA-fs mutation.

FIG. 7 shows the normalized production of 6′SL after 72 hours of cultivation of a strain with the wild type fhuA gene (=reference strain, Ref) or the fhuA::IS2 mutation.

FIG. 8 shows the normalized growth speed of both strains after 72 hours of cultivation of a strain with the wild type fhuA gene (=reference strain, Ref) or the fhuA::IS2 mutation.

FIG. 9 shows the normalized production of LNnT after 72 hours of cultivation of the reference and mutant strains.

FIG. 10 shows the normalized production of 2′FL after 72 hours of cultivation, and the normalized growth speed of the reference and mutant strains where various OMP genes were deleted.

FIG. 11 shows the normalized production of 3FL after 72 hours of cultivation, and the normalized growth speed of the reference and mutant strains where various OMP genes were deleted.

FIG. 12 shows the normalized production of DiFL after 72 hours of cultivation, and the normalized growth speed of the reference and mutant strains where various OMP genes were deleted.

FIG. 13 shows the normalized production of 6′SL after 72 hours of cultivation, and the normalized growth speed of the reference and mutant strains where various OMP genes were deleted.

FIG. 14 shows the normalized production of 3R′SL after 72 hours of cultivation, and the normalized growth speed of the reference and mutant strains where various OMP genes were deleted.

FIG. 15 shows the normalized production of LNnT after 72 hours of cultivation of the reference and mutant strains where various OMP genes were deleted.

FIG. 16 shows the normalized production of LN3 and LNT after 72 hours of cultivation of the reference and mutant strains where various OMP genes were deleted.

### DETAILED DESCRIPTION

In a first range of embodiments, the disclosure provides a transgenic Escherichia coli cell genetically modified to produce at least one bioproduct of the list comprising, preferably including, monosaccharide, phosphorylated monosaccharide, activated monosaccharide, disaccharide, oligosaccharide or glycolipid. The cell comprises an endogenous membrane protein encoding gene that has a reduced expression and/or the endogenous membrane protein encoding gene is mutated. The endogenous membrane protein is any one of a protein as described in table 1. Table 1 further also comprises lists of exemplary genes conforming to the description of the respective membrane protein.

TABLE 1

| | | Example |
|---|---|---|
| Description membrane protein | exemplary genes | in Uniprot |
| outer membrane porin A | Tut, Con, TolG, OmpA, outer membrane protein II*, | P0A910 |
| | polypeptide II*, protein II*, outer membrane protein | |
| | D, outer membrane protein 3a, outer membrane | |
| | protein 0-11, outer membrane protein B | |
| outer membrane porin C | Par, MeoA, outer membrane protein A₂, outer | P06996 |
| | membrane protein Ib, outer membrane protein 4, | |
| | outer membrane protein 0-8, OmpC | |
| outer membrane porin F | NfxB, TolF, Cry, CmlB, outer membrane protein F, | P02931 |
| | colB, outer membrane protein A₁, outer membrane | |
| | protein la, outer membrane protein 4, outer | |
| | membrane protein b, outer membrane protein 0-9, | |
| | OmpF | |
| outer membrane protease VII | OmpT, omptin, protease 7 | P09169 |
| (outer membrane protein 3b) | | |
| cobalamin/cobinamide outer | Cer, Bfe, BtuB | P06129 |
| membrane transporter | | |
| outer membrane channel | WeeA, Toc, Refl, MukA, MtcB, TolC | P02930 |
| maltose outer membrane channel/ | MalL, MalB, LamB | P02943 |
| phage lambda receptor protein | | |
| ferrichrome outer membrane | TonA, FhuA | P06971 |
| transporter/phage receptor | | |
| Ton complex subunit TonB | ExbA, TonB | P02929 |
| long-chain fatty acid outer | Ttr, FadL | P10384 |
| membrane channel/ | | |
| bacteriophage T2 receptor | | |
| nucleoside-specific channel- | NupA, Tsx, nucleoside channel; receptor of phage T6 | P0A927 |
| forming protein | and colicin K | |
| ferric enterobactin outer | Fep, Cbt, Cbr, FeuB, FepA | P05825 |
| membrane transporter | | |
| putative TonB-dependent outer | YncD | P76115 |
| membrane receptor | | |
| outer membrane porin, outer | OmpE, outer membrane pore protein E | P02932 |
| membrane phosphoporin | (E, Ic, NmpAB), PhoE | |
| bacteriophage N4 receptor, outer | NfrA | P31600 |
| membrane protein | | |
| L-methionine/D-methionine | MetD, Abc, metN | P30750 |
| ABC transporter ATP binding | | |
| subunit | | |
| cell division protein FtsX | FtsS, FtsX | P0AC30 |
| cytochrome c menaquinol | TorC | P33226 |
| dehydrogenase TorC | | |
| cytochrome c quinol | YecK, TorY | P52005 |
| dehydrogenase TorY | | |
| soluble lytic murein | SltY, Slt, Slt70 | P0AGC3 |
| transglycosylase | | |
| outer membrane lipoprotein Blc | YjeL, Blc, outer membrane lipoprotein (lipocalin) | P0A901 |
| surface-exposed outer membrane | YaiW | P77562 |
| lipoprotein | | |
| DNA-binding transcriptional | EnvY, envelope protein; thermoregulation of porin | P10805 |
| activator EnvY | biosynthesis | |
| multidrug efflux pump accessory | AcrZ, YbhT, small membrane protein that interacts | P0AAW9 |
| protein AcrZ | with the AcrAB-TolC multidrug efflux pump | |
| adhesin-like autotransporter | YpjA | P52143 |
| YpjA | | |
| inner membrane protein IgaA | YrfF, IgaA | P45800 |
| Type II secretion system protein | YheK, GspL | P45763 |
| GspL | | |
| peptidoglycan | Mgt, YrbM, MtgA | P46022 |
| glycosyltransferase MtgA | | |
| UDP-N- | WecA, Rfe | P0AC78 |
| acetylglucosamine&mdash;undecaprenyl- | | |
| phosphate N- | | |
| acetylglucosaminephosphotransferase | | |
| myristoyl-acyl carrier protein- | WaaN, Mlt, LpxM, MsbB | P24205 |
| dependent acyltransferase | | |
| bifunctional (p)ppGpp | SpoT | P0AG24 |
| synthase/hydrolase SpoT | | |
| GDP/GTP pyrophosphokinase | RelA, stringent factor, ppGpp synthetase I, PSI, | P0AG20 |
| | ppGpp synthase I, (p)ppGpp synthetase I | |
| putative ferritin-like protein | YecI, FtnB | P0A9A2 |
| ferritin iron storage protein | RsgA, FtnA, Ftn | P0A998 |
| phosphatidylglycerophosphatase | PgpB | P0A924 |
| B | | |
| lipoprotein NlpI | YhbM, NlpI | P0AFB1 |
| PF13488 family lipoprotein | YiaD | P37665 |
| YiaD | | |
| murein hydrolase activator NlpD | NlpD, NlpD divisome associated factor; activates | P0ADA3 |
| | peptidoglycan hydrolase | |
| lipoprotein YghG | YghG, GspS_{β} | Q46835 |
| small protein AppX | CbdX, AppX, YccB | P24244 |
| membrane-bound lytic murein | YgdM, Mlt, MltA, Mlt38 | P0A935 |
| transglycosylase A | | |
| outer membrane | PldA, OMPLA, Outer Membrane Phospholipase A | P0A921 |
| phospholipase A | | |
| outer membrane porin family | GusC, UidC | Q47706 |
| protein UidC | | |
| DUF1283 domain-containing | YnfB | P76170 |
| protein YnfB | | |
| putative outer membrane porin | YfeN | P45564 |
| YfeN | | |
| putative fimbrial usher protein | MatD, YagX, ecpC | P77802 |
| EcpC | | |
| inverse autotransporter adhesin | YeeJ | P76347 |
| outer membrane lipoprotein | YbhC | P46130 |
| YhbC | | |
| DUF1471 domain-containing | ComC, YcfR, BhsA, outer membrane protein | P0AB40 |
| multiple stress resistance outer | involved in copper permeability, stress resistance and | |
| membrane protein BhsA | biofilm formation | |
| putative fimbrial usher protein | HtrE | P33129 |
| HtrE | | |
| putative uncharacterized protein | YddL, putative truncated Porin_1 family protein | P77519 |
| YddL | YddL | |
| putative fimbrial usher protein | ElfC, YcbS | P75857 |
| ElfC | | |
| DUF1375 domain-containing | YceK | P0AB31 |
| lipoprotein YceK | | |
| translocation and assembly | TamA, YtfM | P0ADE4 |
| module subunit TamA | | |
| chitobiose outer membrane | ChiP, YbfM | P75733 |
| channel | | |
| metalloprotease LoiP | LoiP, YggG | P25894 |
| Type II secretion | YheF, GspD | P45758 |
| system protein GspD | | |
| lytic murein transglycosylase E | YcgP, SltZ, EmtA, MitE | P0C960 |
| Lipid IV_{A} palmitoyltransferase | YbeG, CrcA, PagP | P37001 |
| acid-inducible putative outer | YdiY | P76206 |
| membrane protein YdiY | | |
| DUF5508 domain-containing | YpjB | P76612 |
| protein YpjB | | |
| DLP12 prophage; putative | RzoD | P58041 |
| prophage lysis lipoprotein RzoD | | |
| peptidoglycan-associated outer | ExcC, protein 21K, Pal - outer membrane lipoprotein | P0A912 |
| membrane lipoprotein Pal | of the Tol-Pal system | |
| putative fimbrial usher protein | SfmD | P77468 |
| SfmD | | |
| outer membrane protein YaiO | YaiO | Q47534 |
| N-acetylmuramoyl-L-alanine | YbjR, AmiD | P75820 |
| amidase D | | |
| putative iron siderophore outer | Fiu, YbiL | P75780 |
| membrane transporter | | |
| EG10155-MONOMER | Cir, FeuA, CirA, colicin I receptor | P17315 |
| inhibitor of c-type lysozyme, | YdhA, MliC | P28224 |
| putative lipoprotein | | |
| putative fimbrial usher protein | YhcD | P45420 |
| YhcD | | |
| polyβ-1,6-N-acetyl-D- | YcdR, HmsF, PgaB | P75906 |
| glucosamine N-deacetylase and | | |
| β-1,6 glycoside hydrolase | | |
| lipopolysaccharide assembly | LptD, YabG, OstA, Imp | P31554 |
| protein LptD | | |
| fimbrial usher domain- | FmlC, YdeT, fimbrial usher protein, C-terminal | P76137 |
| containing protein YdeT | fragment | |
| DUF1190 domain-containing | YgiB | P0ADT2 |
| protein YgiB | | |
| outer membrane protein W | YciD, OmpW | P0A915 |
| outer membrane protein | YzzN, YzzY, EcfK, YaeT, BamA | P0A940 |
| assembly factor BamA | | |
| putative exopolysaccharide | YccZ, GfcE | P0A932 |
| export lipoprotein GfcE | | |
| protein RhsD | RhsD | P16919 |
| copper/silver export system | IbeB, CusC, YlcB | P77211 |
| outer membrane channel | | |
| outer membrane protein | DapX, NlpB, lipoprotein-34, BamC | P0A903 |
| assembly factor BamC | | |
| type I fimbriae usher protein | FimD_1, FimD, FimD_2 | P30130 |
| curli assembly component | CsgF | P0AE98 |
| putative fimbrial usher protein | YehB | P33341 |
| YehB | | |
| outer membrane porin N | YnaG, OmpN | P77747 |
| outer membrane protein | SmqA, small membrane protein A, BamE, SmpA | P0A937 |
| assembly factor BamE | | |
| MltA-interacting protein | YeaF, MipA, scaffolding protein that interacts | P0A908 |
| | with murein polymerase and murein hydrolase | |
| putative fimbrial usher protein | YbgQ | P75750 |
| YbgQ | | |
| starvation lipoprotein | Slp | P37194 |
| outer membrane porin G | OmpG | P76045 |
| cellulose biosynthesis protein | BcsC, YhjL | P37650 |
| BcsC | | |
| protein YjgL | YjgL | P39336 |
| DNA utilization protein HofQ | HopQ, HofQ, protein involved in | P34749 |
| | utilization of DNA as a carbon source | |
| putative multidrug efflux pump | MdtP, YjcP, SdsP | P32714 |
| outer membrane channel | | |
| intermembrane phospholipid | MlaA, VacJ | P76506 |
| transport system - outer | | |
| membrane lipoprotein MlaA | | |
| putative porin YfaZ | YfaZ | P76471 |
| outer membrane polysaccharide | Wza | P0A930 |
| export protein Wza | | |
| partially deacetylated polyβ-1,6- | YcdS, HmsH, PgaA | P69434 |
| N-acetyl-D-glucosamine outer | | |
| membrane porin | | |
| Rac prophage; putative | RzoR | P58042 |
| lipoprotein | | |
| outer membrane lipoprotein | SlyB | P0A905 |
| SlyB | | |
| ferric citrate outer membrane | FecA | P13036 |
| transporter | | |
| lipoprotein YqhH | YqhH | P65298 |
| sensor lipoprotein RcsF | RcsF, RcsF outer membrane lipoprotein - activates | P69411 |
| | the Rcs pathway during envelope stress | |
| outer membrane protein | EcfD, YfiO, BamD | P0AC02 |
| assembly factor BamD | | |
| putative outer membrane porin L | YshA, OmpL | P76773 |
| putative TonB-dependent | YddB | P31827 |
| receptor | | |
| outer membrane lipoprotein - | LpoB, YcfM | P0AB38 |
| activator of MrcB activity | | |
| outer membrane protein YiaT | YiaT | P37681 |
| N-acetylneuraminic acid outer | NanC, YjhA | P69856 |
| membrane channel | | |
| rhs element protein RhsB | RhsB | P16917 |
| protein YzcX | YzcX | P11291 |
| peptidoglycan DD- | YeiV, Spr, MepS | P0AFV4 |
| endopeptidase/peptidoglycan | | |
| LD-carboxypeptidase | | |
| outer membrane protein X | YbiG, OmpP, OmpX | P0A917 |
| carbohydrate-specific outer | YieC, BglH | P26218 |
| membrane porin, cryptic | | |
| outer membrane protein | YfgL, BamB | P77774 |
| assembly factor BamB | | |
| putative fimbrial usher protein | YraJ | P42915 |
| YraJ | | |
| ferric coprogen/ferric | FhuE, ferric coprogen | P16869 |
| rhodotorulic acid outer | outer membrane receptor | |
| membrane transporter | | |
| flagellar L-ring protein | FlaY, FlgH | P0A6S0 |
| flagellar basal-body rod protein | FlaL, FlgG | P0ABX5 |
| FlgG | | |
| rare lipoprotein RlpA | RlpA | P10100 |
| bacteriolytic entericidin B | YjeU, EcnB | P0ADB7 |
| lipoprotein | | |
| LysM domain-containing | YgeR, putative DNA-binding transcriptional | Q46798 |
| putative peptidase lipoprotein | regulator | |
| YgeR | | |
| DLP12 prophage; prophage | Iss, BorD, Bor, YbcU, VboR, lipoprotein bor | P77330 |
| lipoprotein BorD | homolog from lambdoid prophage DLP12 | |
| lipoprotein YfiB | YfiB | P07021 |
| YjbH family protein | YjbH | P32689 |
| translocation and assembly | TamB, YtfO, YtfN | P39321 |
| module subunit TamB | | |
| membrane-bound lytic murein | YafG, MltD, DniR | P0AEZ7 |
| transglycosylase D | | |
| lipoprotein YfgH | YfgH | P65290 |
| putative lipoprotein GfcD | YmcA, GfcD | P75882 |
| SoxR [2Fe—2S] reducing system | YdgP, RsxG | P77285 |
| protein RsxG | | |
| entericidin A lipoprotein, | EcnA | P0ADB4 |
| antidote to entericidin B | | |
| polyphosphate kinase | Ppk | P0A7B1 |
| intermembrane transport | PqiC, YmbA | P0AB10 |
| lipoprotein PqiC | | |
| putative invasin YchO | YchO, YchP | P39165 |
| murein lipoprotein | MlpA, Lpp | P69776 |
| outer membrane lipoprotein | QseG, YfhG | P0AD44 |
| QseG | | |
| periplasmic chaperone Skp | Skp, OmpH, HlpA, HLP-I | P0AEU7 |
| lipopolysaccharide transport | YhbN, LptA | P0ADV1 |
| system protein LptA | | |
| membrane-bound lytic murein | MitB, Slt, Slt35 | P41052 |
| transglycosylase B | | |
| outer membrane lipoprotein | YchC, LolB, HemM | P61320 |
| LolB | | |
| aldose sugar dehydrogenase YliI | YliI | P75804 |
| curli transport specificity factor | CsgE | P0AE95 |
| CP4-44 prophage; self | Agn, YeeQ, YzzX, Flu, Ag43 | P39180 |
| recognizing antigen 43 (Ag43) | | |
| autotransporter | | |
| curli secretion channel | CsgG | P0AEA2 |
| lipopolysaccharide assembly | LptE, RlpB | P0ADC1 |
| protein LptE | | |
| divisome-associated lipoprotein | EcfH, YraP | P64596 |
| YraP | | |
| membrane-bound lytic murein | YggZ, MltC | P0C066 |
| transglycosylase C | | |
| membrane-bound lytic murein | YfhD, MltF | P0AGC5 |
| transglycosylase F | | |
| outer membrane lipoprotein - | LpoA, YraM | P45464 |
| activator of MrcA activity | | |
| putative autotransporter adhesin | YfaJ, YfaK, YfaF, YfaL | P45508 |
| YfaL | | |
| curlin, minor subunit | CsgB | P0ABK7 |

In further embodiments, the disclosure provides a method for conferring bacteriophage resistance in an E. coli cell. First, an E. coli cell that is genetically modified to produce at least one bioproduct as described herein is provided. At least one endogenous membrane protein encoding gene of the cell is mutated and/or has a reduced expression. The membrane protein is any one of a protein as described in Table 1.

The disclosure also provides a method for producing at least one bioproduct as described herein with an E. coli cell. First, an E. coli cell that is genetically modified to produce at least one bioproduct as described herein is provided. At least one endogenous membrane protein encoding gene of the cell has been mutated and/or has a reduced expression. The membrane protein is any one of a protein as described in Table 1. The cell is cultivated in a medium under conditions permissive for the production of the desired bioproduct. Preferably, the bioproduct is separated from the cultivation. More preferably, the bioproduct is purified after separation from the cultivation.

In a further embodiment, the disclosure provides a method for increasing the production of at least one bioproduct as described herein with an E. coli cell that is genetically modified to produce at least one bioproduct as compared to an E. coli cell genetically modified to produce the bioproduct(s) but lacking the extra reduced expression and/or mutation described hereafter. An E. coli cell that is genetically modified to produce at least one bioproduct is further altered by providing a mutation in and/or a reduced expression of an endogenous membrane protein encoding gene. The cell is cultivated in a medium under conditions permissive for the production of the desired bioproduct. Preferably, the bioproduct is separated from the cultivation. The bioproduct can also be purified as described herein. The membrane protein is any one of the proteins as described in Table 1.

According to the disclosure, Escherichia coli (abbreviated herein as E. coli) can be, but not limited to, Escherichia coli B, Escherichia coli BL21, Escherichia coli C, Escherichia coli W, Escherichia coli Nissle, Escherichia coli K12. More specifically, the latter term relates to cultivated Escherichia coli strains—designated as E. coli K12 strains—that are well-adapted to the laboratory environment, and, unlike wild type strains, have lost their ability to thrive in the intestine. Well-known examples of the E. coli K12 strains are K12 Wild type, W3110, MG1655, JM109, DH1, M182, MC1000, MC1060, MC1061, MC4100, JM101, NZN111 and AA200. Hence, the disclosure preferably relates to a mutated and/or transformed Escherichia coli strain as indicated above wherein the E. coli strain is a K12 strain. More preferably, the disclosure relates to a mutated and/or transformed Escherichia coli strain as indicated above wherein the K12 strain is E. coli MG1655.

In a further embodiment, the membrane protein is chosen from the list comprising: COG groups COG4206, COG2067, COG4771, COG1629, COG4580, COG2885, COG3203, COG4571, COG1538, COG3248, COG0810, COG0457; an outer membrane porin, an outer membrane protease 7, a cobalamin/cobinamide outer membrane transporter, an outer membrane channel, a maltose outer membrane channel, a ferrichrome outer membrane transporter, a Ton complex subunit, a long-chain fatty acid outer membrane channel, a nucleoside-specific channel-forming protein, a ferric enterobactin outer membrane transporter, a putative TonB-dependent outer membrane receptor, an outer membrane protein, a phage receptor.

Preferably, the membrane protein is chosen from the list comprising, more preferably consisting, of: OmpA (SEQ ID NO: 2), OmpC (SEQ ID NO: 4), OmpF (SEQ ID NO: 6), OmpT (SEQ ID NO: 8), BtuB (SEQ ID NO: 10), TolC (SEQ ID NO: 12), LamB (SEQ ID NO: 14), FhuA (SEQ ID NO: 16), TonB (SEQ ID NO: 18), FadL (SEQ ID NO: 20), Tsx (SEQ ID NO: 22), FepA (SEQ ID NO: 24), YncD (SEQ ID NO: 26), PhoE (SEQ ID NO: 28), and NfrA (SEQ ID NO: 30), a functional homolog of any one of SEQ ID NOs: 2, 4, 6, 8, 10, 12, 14, 16, 18, 20, 22, 24, 26, 28, 30, or a membrane protein having at least 70% sequence identity to the full length amino acid sequence of any one of SEQ ID NOs: 2, 4, 6, 8, 10, 12, 14, 16, 18, 20, 22, 24, 26, 28, 30.

As used herein, a membrane protein having an amino acid sequence having at least 70% sequence identity to any of the enlisted membrane proteins, is to be understood as that the sequence has 70%, 71%, 72%, 73%, 74%, 75% 76%, 77% 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length of the amino acid sequence of the respective membrane protein.

The amino acid sequence of such membrane protein can be a sequence chosen from SEQ ID NOs: 2, 4, 6, 8, 10, 12, 14, 16, 18, 20, 22, 24, 26, 28 or 30 of the attached sequence listing, a functional homolog of any one of SEQ ID NOs: 2, 4, 6, 8, 10, 12, 14, 16, 18, 20, 22, 24, 26, 28, 30, or an amino acid sequence that has at least 70% sequence identity, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length amino acid sequence of any one of SEQ ID NOs: 2, 4, 6, 8, 10, 12, 14, 16, 18, 20, 22, 24, 26, 28 or 30.

According to a preferred embodiment of this disclosure, the mutation and/or reduced expression of the membrane protein encoding gene confers bacteriophage resistance to a bacteriophage selected from the bacteriophage families listed in table 2.

TABLE 2
bacteriophage families and their receptors

| Phage | Family | Main host | Receptor | References |
|---|---|---|---|---|
| 434 | Siphoviridae | Escherichia | OmpC | Hantke 1978 |
| | | coli | | |
| BF23 | Siphoviridae | Escherichia | BtuB | Bradbeer, Woodrow and Khalifah 1976 |
| | | coli | | |
| K3 | Myoviridae | Escherichia | OmpA | Skurray, Hancock and Reeves 1974; |
| | | coli | | Manning and Reeves 1976; Van Alphen, |
| | | | | Havekes and Lugtenberg 1977 |
| K10 | Siphoviridae | Escherichia | LamB | Roa 1979 |
| | | coli | | |
| Me1 | Myoviridae | Escherichia | OmpC | Verhoef, de Graaff and Lugtenberg 1977 |
| | | coli | | |
| M1 | Myoviridae | Escherichia | OmpA | Hashemolhosseini et al. 1994 |
| | | coli | | |
| Ox2 | Myoviridae | Escherichia | OmpA | Morona and Henning 1984 |
| | | coli | | |
| TLS | Siphoviridae | Escherichia | TolC | German and Misra 2001 |
| | | coli | | |
| TuIa | Myoviridae | Escherichia | OmpF | Datta, Arden and Henning 1977 |
| | | coli | | |
| TuIb | Myoviridae | Escherichia | OmpC | Datta, Arden and Henning 1977 |
| | | coli | | |
| TuII* | Myoviridae | Escherichia | OmpA | Datta, Arden and Henning 1977 |
| | | coli | | |
| T1 | Siphoviridae | Escherichia | FhuA // | Hantke and Braun 1975; Hancock and |
| | | coli | TonB | Braun 1976; Hantke and Braun 1978 |
| T2 | Myoviridae | Escherichia | OmpF // | Hantke 1978; Morona and Henning 1986; |
| | | coli | FadL | Black 1988 |
| T4 | Myoviridae | Escherichia | OmpC | Prehm et al. 1976; Mutoh, Furukawa and |
| | | coli | | Mizushima 1978; Goldberg, Grinius and |
| | | | | Letellier 1994 |
| T5 | Siphoviridae | Escherichia | FhuA | Braun, Schaller and Wolff 1973; Braun |
| | | coli | | and Wolff 1973; Heller and Braun 1982 |
| T6 | Myoviridae | Escherichia | Tsx | Manning and Reeves 1976; Manning and |
| | | coli | | Reeves 1978 |
| λ | Siphoviridae | Escherichia | LamB | Randall-Hazelbauer and Schwartz 1973 |
| | | coli | | |
| φ80 | Siphoviridae | Escherichia | FhuA // | Hantke and Braun 1975; Wayne and |
| | | coli | TonB | Neilands 1975; Hancock and Braun 1976; |
| | | | | Hantke and Braun 1978 |
| TC45 | | Escherichia | PhoE | Chai and Foulds, 1978 (PMID: 97266) |
| TC23 | | coli | | |
| T2 | Myoviridae | Escherichia | OmpT | Hashemolhosseini et al, 1994 (PMID: |
| | | coli | | 8027994) |
| N4 | | Escherichia | NfrA | McPartland et al, 2009 (PMID: 19011026) |
| | | coli | | |
| H8 | Siphoviridae | Escherichia | FepA | Rabsch et al, 2007 (PMID: 17526714) |
| | | coli | | |
| IME253 | | Escherichia | FepA | Li et al, 2019 (PMID: 31105661) |
| | | coli | | |
| IME347 | | Escherichia | YncD | Li et al, 2019 (PMID: 31105661); Li et al, |
| (T1-like) | | coli | | 2018 (PMID: 30146706) |

According to specific embodiments, the bacteriophage resistance is characterized by at least one of:
(a) not causing an abortive bacteriophage infection;
(b) preventing phage genomic replication in an E. coli cell;
(c) preventing phage lysogeny in an E. coli cell;
(d) reducing and/or abolishing the bacteriophage binding capacity of the membrane protein;
(e) not impairing bioproduct production;
(f) enhancing bioproduct production;
(g) enhancing productivity in a fermentation
(h) not impairing growth or growth speed of the cells;
(i) enhancing growth or growth speed of the cells;
(j) not impairing biomass production in a fermentation using the cell;
(k) enhancing biomass production in a fermentation using the cell; and/or
(l) reducing biomass production in a fermentation using the cell; each possibility represents a separate embodiment of the disclosure.

The functional phage resistance may be characterized by one, two, three, four, five, six, seven, eight, nine, ten, eleven or all of (a)-(l).

According to specific embodiments, the functional phage resistance is characterized by at least (a)+(b), (a)+(c), (a)+(d), (a)+(e), (a)+(f), (a)+(g), (a)+(h), (a)+(i), (a)+(j), (a)+(k), (a)+(l), (b)+(c), (b)+(d), (b)+(e), (b)+(f), (b)+(g), (b)+(h), (b)+(i), (b)+(j), (b)+(k), (b)+(l), (c)+(d), (c)+(e), (c)+(f), (c)+(g), (c)+(h), (c)+(i), (c)+(j), (c)+(k), (c)+(l), (d)+(e), (d)+(f), (d)+(g), (d)+(h), (d)+(i), (d)+(j), (d)+(k), (d)+(l), (e)+(f), (e)+(g), (e)+(i), (e)+(j), (e)+(k), (e)+(l), (f)+(g), (f)+(h), (f)+(i), (f)+(j), (f)+(k), (f)+(l), (g)+(h), (g)+(i), (g)+(j), (g)+(k), (g)+(l), (i)+(j), (i)+(k), (i)+(l), (j)+(k), (j)+(l), and/or (k)+(l).

According to specific embodiments, the functional phage resistance system is characterized by at least (d)+(e), (d)+(f), (d)+(g), (d)+(h), (d)+(i), (d)+(j), (d)+(k), and/or (d)+(l).

According to a specific embodiment, the functional phage resistance system is characterized by (d)+(f)+(g), (d)+(g)+(i), (d)+(g)+(k), (d)+(f)+(j), (d)+(g)+(l), (d)+(f)+(k), (d)+(f)+(l), (d)+(e)+(i), (d)+(e)+(k), (d)+(e)+(h)+(j), (d)+(f)+(h)+(k).

According to a specific embodiment, the functional phage resistance system is characterized by (d)+(e)+(h)+(j), (d)+(f)+(g)+(i), (d)+(g)+(e)+(k)+(i), (d)+(g)+(f)+(i)+(l), (d)+(g)+(e)+(k), (d)+(g)+(f)+(l).

In some embodiments of the disclosure, the mutation and/or reduced expression of the membrane protein encoding gene confers unaffected bioproduct production wherein similar or the same levels of bioproduct are produced as is produced by a cell having the same genetic make-up but lacking the modified expression of the membrane protein encoding gene. Similar or the same levels of bioproduct produced is to be understood to be at least 75% of the levels of bioproduct as produced by a cell having the same genetic make-up but lacking the modified expression of the membrane protein encoding gene. A production of at least 75% is to be understood as to be 75%, 76%, 77%, 78%, 79%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9%, 100% of the levels produced by a cell having the same genetic make-up but lacking the modified expression of the membrane protein encoding gene. Preferably, the mutation and/or reduced expression of the membrane protein encoding gene confers enhanced bioproduct formation in or by the cell wherein the cell produces more bioproduct in comparison to a cell having the same genetic make-up but lacking the mutation and/or reduced expression of the membrane protein encoding gene. In some other embodiments of the disclosure, the mutation and/or reduced expression of the membrane protein encoding gene confers unaffected cell growth, or cell growth speed, productivity and/or biomass production wherein similar or the same levels of cell growth speed and/or biomass is produced as the cell growth speed, productivity and or biomass produced by a cell having the same genetic make-up but lacking the mutation and/or reduced expression of the membrane protein encoding gene. Preferably, the mutation and/or reduced expression of the membrane protein encoding gene confers enhanced cell growth speed, productivity and/or biomass production in or by the cell wherein the cell produces more biomass, has a higher productivity and/or has an enhanced cell growth speed in comparison to a cell having the same genetic make-up but lacking the mutation and/or reduced expression of the membrane protein encoding gene.

According to some embodiments of the disclosure, the mutation and/or reduced expression of the membrane protein encoding gene confers reduced and/or abolished bacteriophage binding capacity of the membrane protein and/or to the cell.

According to specific embodiments of the disclosure, the reduced expression of the membrane protein encoding gene comprises any one or more of:
i) mutating the transcription unit of the membrane protein encoding gene;
ii) mutating the endogenous/homologous promoter of the membrane protein encoding gene;
iii) mutating the ribosome binding site of the membrane protein encoding gene;
iv) mutating an UTR of the membrane protein encoding gene; and/or
v) mutating the transcription terminator.

In some embodiments of the disclosure, the mutation of the membrane protein encoding gene is a point mutation. Such point mutation can result in either i) a membrane protein of the same length; ii) a shorter membrane protein due to the mutation creating a premature stop codon in the membrane protein encoding gene; iii) a shorter membrane protein being a fragment as defined herein; or iv) a longer membrane protein due to the mutation changing the normal stop codon to a codon coding for an amino acid and translation continuing till the next stop.

In some embodiments of the disclosure, the mutation of the membrane protein encoding gene renders the membrane protein shorter. This can be obtained by i) a point-mutation due to the mutation creating a premature stop codon in the membrane protein encoding gene, ii) other mutations creating a premature stop codon in the membrane protein encoding gene, iii) a fragment as defined herein, or iv) deletion of part of the membrane protein encoding gene's polynucleotide sequence. Such shorter proteins in some instances result in the same phenotype as a knock-out mutant.

In some embodiments of the disclosure, the mutation of the membrane protein encoding gene completely knocks out the membrane protein encoding gene to be obtained in ways as known by the person skilled in the art.

In other embodiments of the disclosure, the mutation of the membrane protein encoding gene renders the membrane protein longer. This can be obtained by an insertion or a C- or N-terminal addition of at least one base in the membrane protein encoding gene. Preferably, the mutation confers an insertion or addition of at least 2 amino acids into the encoded membrane protein's amino acid sequence. More preferably the mutation confers an insertion of more than 2, 2,3,4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 amino acids. Even more preferred, the mutation confers an insertion ranging between 15 and 45 amino acids, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 amino acids. Preferably, the insertion extends the extracellular loops in the 3 dimensional space of the protein, and that mutation confers resistance to any bacteriophage that is able to infect the cell by binding to the phage receptor protein. Preferably, the mutation does not decrease i) bioproduct production; ii) growth of the cell, iii) productivity and/or iv) biomass production. More preferably, the mutation increases and/or enhances i) bioproduct production; ii) growth of the cell, iii) productivity and/or iv) biomass production.

According to the disclosure, the mutation of the membrane protein encoding gene is any one of an in-frame mutation, an out-of-frame mutation or a partial or complete knock-out mutation.

In a preferred embodiment, a cell is provided according to the disclosure, wherein the mutation occurs in a tolC (SEQ ID NO: 12) encoding gene or a gene encoding a functional homolog of SEQ ID NO: 12 or a gene encoding a protein having at least 70% sequence identity of the full length of SEQ ID NO: 12, and wherein the mutation comprises an 11 amino acid duplication of the amino acid sequence VGLSFSLPIYQ (SEQ ID NO: 31).

In a further preferred embodiment, the cell and/or the method comprises at least two endogenous membrane protein encoding genes that are mutated and/or have a reduced expression. The endogenous membrane proteins are at least any two of the proteins as described in table 1. More preferably, at least 2,2,3,4,5,6,7,8,9, 10, 11, 12, 13, 14, 15, 16, 17, 18, 19, 20, 21, 22, 23, 24, 25 endogenous membrane protein encoding genes are mutated and/or have a reduced expression.

It is to be understood that a person skilled in the art will, upon reading this disclosure, be able to identify any other mutation to the membrane protein encoding gene, such as the exemplary publicly available mutations as listed in Table 3. However, not all of the mutations will prove useful in the production of the bioproduct by the genetically modified cell. The skilled person will however learn from the disclosure which membrane proteins and what kind of mutation (knock out, elongation, truncation, fragment of the protein) and/or reduced expression will provide unimpaired, or even enhanced i) bioproduct production; ii) growth of the cell, iii) productivity and/or iv) biomass production, when compared to production by a cell having the same genetic make-up but lacking the mutation and/or reduced expression in the membrane protein encoding gene.

TABLE 3
mutations conferring resistance to bacteriophage

| | | Resistance to | |
|---|---|---|---|
| omp | mutation | phage families | reference |
| tolC | full_deletion | TLS | German and Misra et al, 2001 |
| | | | (PMID: 11350161) |
| tolC | Insertion_AA73 (YS) | TLS | German and Misra et al, 2001 |
| | | | (PMID: 11350161) |
| tolC | Insertion_AA99 | TLS | German and Misra et al, 2001 |
| | (SYRDANGINSNATSASLOLTQSIF.) | | (PMID: 11350161) |
| tolC | Deletion_AA80-86 | TLS | German and Misra et al, 2001 |
| | | | (PMID: 11350161) |
| tolC | G75V | TLS | German and Misra et al, 2001 |
| | | | (PMID: 11350161) |
| tolC | S279P | TLS | German and Misra et al, 2001 |
| | | | (PMID: 11350161) |
| tolC | G302C | TLS | German and Misra et al, 2001 |
| | | | (PMID: 11350161) |
| tolC | G302D | TLS | German and Misra et al, 2001 |
| | | | (PMID: 11350161) |
| tolC | Q303P | TLS | German and Misra et al, 2001 |
| | | | (PMID: 11350161) |
| tolC | Deletion_AA295-303, N304D | TLS | German and Misra et al, 2001 |
| | | | (PMID: 11350161) |
| tolC | Y283D | TLS | German and Misra et al, 2001 |
| | | | (PMID: 11350161) |
| fepA | full_deletion | H8 | Rabsch et al, 2007 |
| | | | (PMID: 17526714) |
| tonB | full_deletion | H8 | Rabsch et al, 2007 |
| | | | (PMID: 17526714) |
| fepA | Deletion_AA1-150 | H8 | Rabsch et al, 2007 |
| | | | (PMID: 17526714) |
| fepA | Deletion_AA199-206 | H8 | Rabsch et al, 2007 |
| | | | (PMID: 17526714) |
| fepA | Deletion_AA315-326 | H8 | Rabsch et al, 2007 |
| | | | (PMID: 17526714) |
| fepA | Deletion_AA467-497 | H8 | Rabsch et al, 2007 |
| | | | (PMID: 17526714) |
| fepA | Deletion_AA592-603 | H8 | Rabsch et al, 2007 |
| | | | (PMID: 17526714) |
| fepA | Deletion_AA681-708 | H8 | Rabsch et al, 2007 |
| | | | (PMID: 17526714) |
| fepA | R313A R316A | H8 | Rabsch et al, 2007 |
| | | | (PMID: 17526714) |
| fepA | Y260A F329A | H8 | Rabsch et al, 2007 |
| | | | (PMID: 17526714) |
| fepA | Y260A Y272A | H8 | Rabsch et al, 2007 |
| | | | (PMID: 17526714) |
| yncD | full_deletion | T1-like | Li et al, 2018 |
| | | (IME347) | (PMID: 30146706) |
| fhuA | Deletion_AA675-704 | IME18 | Li et al, 2019 |
| | | | (PMID: 31105661) |
| fhuA | T629-fs | IME18 | Li et al, 2019 |
| | | | (PMID: 31105661) |
| fhuA | F519-fs | IME18 | Li et al, 2019 |
| | | | (PMID: 31105661) |
| fepA | full_deletion | IME253 | Li et al, 2019 |
| | | | (PMID: 31105661) |
| ompF | Deletion_AA79-128 | IME281 | Li et al, 2019 |
| | | | (PMID: 31105661) |
| ompF | D76-fs | IME281 | Li et al, 2019 |
| | | | (PMID: 31105661) |
| ompF | full_deletion | IME281 | Li et al, 2019 |
| | | | (PMID: 31105661) |
| tsx | F18-fs | IME339 | Li et al, 2019 |
| | | | (PMID: 31105661) |
| tsx | L149E | IME339 | Li et al, 2019 |
| | | | (PMID: 31105661) |
| tsx | full_deletion | IME339 | Li et al, 2019 |
| | | | (PMID: 31105661) |
| ompA | V122-fs | IME340 | Li et al, 2019 |
| | | | (PMID: 31105661) |
| ompA | full_deletion | IME340 | Li et al, 2019 |
| | | | (PMID: 31105661) |
| ompA | Q38* | IME340 | Li et al, 2019 |
| | | | (PMID: 31105661) |
| fadL | D34* | IME341 | Li et al, 2019 |
| | | | (PMID: 31105661) |
| fadL | L161V | IME341 | Li et al, 2019 |
| | | | (PMID: 31105661) |
| fadL | L394E | IME341 | Li et al, 2019 |
| | | | (PMID: 31105661) |
| yncD | full_deletion | IME347 | Li et al, 2019 |
| | | | (PMID: 31105661) |
| LamB | G43V | Lambda | Chatterjee and Rothenberg, 2012 |
| | | | (PMID: 23202520) |
| LamB | E173K | Lambda | Chatterjee and Rothenberg, 2012 |
| | | | (PMID: 23202520) |
| LamB | G176D | Lambda | Chatterjee and Rothenberg, 2012 |
| | | | (PMID: 23202520) |
| LamB | S177F | Lambda | Chatterjee and Rothenberg, 2012 |
| | | | (PMID: 23202520) |
| LamB | S179F | Lambda | Chatterjee and Rothenberg, 2012 |
| | | | (PMID: 23202520) |
| LamB | F180S | Lambda | Chatterjee and Rothenberg, 2012 |
| | | | (PMID: 23202520) |
| LamB | Y188D | Lambda | Chatterjee and Rothenberg, 2012 |
| | | | (PMID: 23202520) |
| LamB | T189P | Lambda | Chatterjee and Rothenberg, 2012 |
| | | | (PMID: 23202520) |
| LamB | G270R | Lambda | Chatterjee and Rothenberg, 2012 |
| | | | (PMID: 23202520) |
| LamB | G270V | Lambda | Chatterjee and Rothenberg, 2012 |
| | | | (PMID: 23202520) |
| LamB | S272L | Lambda | Chatterjee and Rothenberg, 2012 |
| | | | (PMID: 23202520) |
| LamB | G274D | Lambda | Chatterjee and Rothenberg, 2012 |
| | | | (PMID: 23202520) |
| LamB | S275F | Lambda | Chatterjee and Rothenberg, 2012 |
| | | | (PMID: 23202520) |
| LamB | F284V | Lambda | Chatterjee and Rothenberg, 2012 |
| | | | (PMID: 23202520) |
| LamB | G407D | Lambda | Chatterjee and Rothenberg, 2012 |
| | | | (PMID: 23202520) |
| LamB | G407V | Lambda | Chatterjee and Rothenberg, 2012 |
| | | | (PMID: 23202520) |
| LamB | G426D | Lambda | Chatterjee and Rothenberg, 2012 |
| | | | (PMID: 23202520) |
| PhoE | R179H | TC45 | Korteland et al, 1985 |
| | | | (PMID: 2414105) |
| ompF | full_deletion | bacteriophage | Traurig and Misra, 1999 |
| | | K20 | (PMID: 10564794) |
| ompF | Deletion_AA222-229 | bacteriophage | Traurig and Misra, 1999 |
| | | K20 | (PMID: 10564794) |
| ompF | N264K | bacteriophage | Traurig and Misra, 1999 |
| | | K20 | (PMID: 10564794) |
| ompF | G271R | bacteriophage | Traurig and Misra, 1999 |
| | | K20 | (PMID: 10564794) |
| ompF | G307D | bacteriophage | Traurig and Misra, 1999 |
| | | K20 | (PMID: 10564794) |
| ompF | G307D G69D | bacteriophage | Traurig and Misra, 1999 |
| | | K20 | (PMID: 10564794) |
| ompF | L227F | bacteriophage | Traurig and Misra, 1999 |
| | | K20 | (PMID: 10564794) |
| ompF | G271S | bacteriophage | Traurig and Misra, 1999 |
| | | K20 | (PMID: 10564794) |
| ompF | G307S | bacteriophage | Traurig and Misra, 1999 |
| | | K20 | (PMID: 10564794) |
| ompF | G307R | bacteriophage | Traurig and Misra, 1999 |
| | | K20 | (PMID: 10564794) |
| btuB | Deletion_AA464-468 | phage BF23 | Full-Schaefer et al, 2005 |
| | | | (PMID: 15716445) |
| btuB | Deletion_AA514-519 | phage BF23 | Full-Schaefer et al, 2005 |
| | | | (PMID: 15716445) |
| btuB | Deletion_AA353-358 | phage BF23 | Full-Schaefer et al, 2005 |
| | | | (PMID: 15716445) |
| ompA | G175D | Ox2, Ox4, Ox5, | Morona et al, 1985 |
| | | M1, Ox2h5, | (PMID: 3902787) |
| | | Ox2h20 | |
| ompA | G175S | Ox2, Ox4, Ox5, | Morona et al, 1985 |
| | | Ox2h5, | (PMID: 3902787) |
| | | Ox2h20 | |
| ompA | G49V | Ox2, Ox4, Ox5, | Morona et al, 1985 |
| | | Ox2h20 | (PMID: 3902787) |
| ompA | G91D | Tull*-46, | Morona et al, 1985 |
| | | Tull*-60, | (PMID: 3902787) |
| | | Tull*-24, | |
| | | Tull*-6, K3, | |
| | | K4, K5, Ac3, | |
| | | Ox3, Tull*-26, | |
| | | Ox2, Ox4, Ox5, | |
| | | Ox2h20 | |
| ompA | G86D | Tull*- 46, | Morona et al, 1985 |
| | | Tull*- 60, | (PMID: 3902787) |
| | | Tull*- 6, Ox2, | |
| | | Ox4, Ox5 | |
| ompA | S129F | Tull*-60, | Morona et al, 1985 |
| | | Tull*-6, Ac3, | (PMID: 3902787) |
| | | Ox3 | |
| ompA | S129P | Tull*-46, | Morona et al, 1985 |
| | | Tull*-60, | (PMID: 3902787) |
| | | Tull*-6, Ac3, | |
| | | Ox3, Ox2, Ox4, | |
| | | Ox5, M1 | |
| ompA | V131D | Tull*-46, | Morona et al, 1985 |
| | | Tull*-60, | (PMID: 3902787) |
| | | Tull*-24, | |
| | | Tull*-6, K3, | |
| | | K4, K5, Ac3, | |
| | | Ox3, Tull*-26 | |
| ompA | Deletion_AA89 | Tull*-46, | Morona et al, 1985 |
| | | Tull*-60, | (PMID: 3902787) |
| | | Tull*-24, | |
| | | Tull*-6, K3, | |
| | | K4, K5, K3h1, | |
| | | Ac3, Ox3, | |
| | | Tull*-26, Ox2, | |
| | | Ox4, Ox5, | |
| | | Ox2h5, | |
| | | Ox2h20 | |
| ompA | E89K | Tull*-46, | Morona et al, 1985 |
| | | Tull*-60, | (PMID: 3902787) |
| | | Tull*-24, | |
| | | Tull*-6, K3, | |
| | | K4, K5, K3h1, | |
| | | Ac3, Ox3, | |
| | | Tull*-26, Ox2, | |
| | | Ox4, Ox5, | |
| | | Ox2h5, | |
| | | Ox2h20 | |
| ompA | I45N | Tull*-60, Ox2, | Morona et al, 1985 |
| | | Ox4, Ox5 | (PMID: 3902787) |
| ompA | G91V | Tull*-46, | Morona et al, 1985 |
| | | Tull*-60, | (PMID: 3902787) |
| | | Tull*-24, | |
| | | Tull*-6, K3, | |
| | | K4, K5, Ac3, | |
| | | Ox3, Tull*-26, | |
| | | Ox2, Ox4, Ox5, | |
| | | Ox2h5, | |
| | | Ox2h20 | |
| ompA | G91C | Tull*-60, Ac3, | Morona et al, 1985 |
| | | Ox3, Ox2, Ox4, | (PMID: 3902787) |
| | | Ox5, Ox2h20 | |
| fhuA | Deletion_AA381 | T1, T5, phi80 | Killmann et al, 1992 |
| | | | (PMID: 1534324) |
| fhuA | Deletion_AA364-374 | T1, T5, phi80 | Killmann et al, 1995 |
| | | | (PMID: 7836303) |
| fhuA | Deletion_AA380-386 | T1, T5, phi80 | Killmann et al, 1995 |
| | | | (PMID: 7836303) |
| fhuA | Deletion_AA349-358 | phi80 | Killmann et al, 1995 |
| | | | (PMID: 7836303) |
| ompC | Deletion_AA172-200 | ? | Vakharia and Misra, 1996 |
| | | | (PMID: 8820656) |
| tsx | N276Y | T6, H1, H3, H8, | Maier et al, 1990 |
| | | K9, K18, Ox1 | (PMID: 2199819) |
| tsx | full_deletion | T6, T6h3.1, | Schneider et al, 1993 |
| | | Ox1, H1, H3, | (PMID: 8491700) |
| | | H8, K18 | |
| tsx | N271K | (T6), T6h3.1, | Schneider et al, 1993 |
| | | Ox1, H1, H3, | (PMID: 8491700) |
| | | H8, K18 | |
| tsx | N276K | (T6), Ox1, H1, | Schneider et al, 1993 |
| | | H3, H8, K18 | (PMID: 8491700) |
| tsx | Deletion_AA261-266 | T6, T6h3.1, | Schneider et al, 1993 |
| | | Ox1, H1, H3, | (PMID: 8491700) |
| | | H8, K18 | |
| tsx | Duplication_AA251-259 | (T6), T6h3.1, | Schneider et al, 1993 |
| | | Ox1, H1, H3, | (PMID: 8491700) |
| | | H8, K18 | |

According to this disclosure, the cell is genetically modified for the production of at least one bioproduct. Such bioproduct can be a monosaccharide, a phosphorylated monosaccharide, an activated monosaccharide, a disaccharide, an oligosaccharide or a glycolipid.

In some embodiments, the bioproduct is a monosaccharide as described herein. Preferably, the monosaccharide is selected from the group comprising glucosamine, mannose, xylose, N-acetylmannosamine, N-acetylneuraminic acid, N-glycolylneuraminic acid, sialic acid, N-acetylgalactosamine, galactosamine, fucose, rhamnose, glucuronic acid, gluconic acid.

In some embodiments, the bioproduct is a phosphorylated monosaccharide as described herein.

Preferably, the phosphorylated monosaccharide is selected from the group comprising glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate.

In other embodiments of the disclosure, the bioproduct is an activated monosaccharide as described herein. Preferably, the activated monosaccharide is selected from the group comprising GDP-fucose, UDP-glucose, UDP-galactose, UDP-N-acetylglucosamine, CMP-sialic acid.

In other embodiments of this disclosure, the bioproduct is a disaccharide as described herein. Preferably, such disaccharide is lactose or N-acetyllactosamine (LacNAc). An example of fermentative production of lactose by the cell is provided in the examples. Fermentative production of LacNAc is possible by feeding the cell N-acetyllactosamine (GlcNAc) as described by Ruffing and Chen, Microb Cell Fact. 2006, 5: 25.

In some embodiments of this disclosure, the bioproduct is an oligosaccharide as defined herein. Preferably, the oligosaccharide is selected from the group of fucosyllactoses, sialyllactoses, Lacto-N-tetraoses, difucosyllacto-N-tetraose, sialyl-lacto-N-tetraoses, lacto-N-fucopentaoses, lewis-type antigens. More preferably, the oligosaccharide is selected from the group comprising 2′FL, 3FL, DiFL, Lacto-N-triose, LNT, LNnT, 3′SL, 6′SL, LSTa, LSTb, LSTc, LSTd, DFLNT, lacto-N-fucopentaose I, lacto-N-fucopentaose II, lacto-N-fucopentaose III, lacto-N-fucopentaose V, lacto-N-fucopentaose VI, H1 antigen, Lewis^{a}, Lewis^{b}, sialyl Lewis^{a}, H2 antigen, Lewis^{x}, Lewis^{y}; sialyl-Lewis^{X}. Examples of cells enabled to produce such oligosaccharides are described herein.

In other embodiments, the bioproduct is a glycolipid as described herein.

In one embodiment, the E. coli cell is transformed with at least one heterologous gene to produce a sialic acid pathway or sialylation pathway, or fucosylation pathway or galactosylation pathway or N-acetylglucosamine carbohydrate pathway. This cell is transformed by introduction of a heterologous gene, genetic cassette or set of genes as described in the art.

A further embodiment of the disclosure provides a method to produce a fucosylated, sialylated, galactosylated oligosaccharide, N-acetylglucosamine containing oligosaccharide, or sialic acid with a cell as described herein, respectively.

In one embodiment of the disclosure, the methods as described herein are producing the bioproduct LNnT and the membrane protein is preferably any one or more of LamB (SEQ ID NO: 14), FhuA (SEQ ID NO: 16), FadL (SEQ ID NO: 20), and NfrA (SEQ ID NO: 30), a functional homolog of any one of SEQ ID NO: 14, 16, 20 or 30, or a membrane protein having at least 70% sequence identity to the full length amino acid sequence of any one of SEQ ID NOs: 14, 16, 20, 30 and wherein preferably the mutation results in a knock-out phenotype of the gene.

In another embodiment of the disclosure, the methods as described herein are producing sialyllactose, preferably 6′SL, and preferably the membrane protein is FhuA (SEQ ID NO: 16), a functional homolog of SEQ ID NO: 16, or a membrane protein having at least 70% sequence identity to the full length amino acid sequence of SEQ ID NO: 16. Preferably, the mutation results in a knock-out phenotype of the gene.

In a further embodiment, the disclosure provides for the use of a cell as described herein for the production of a bioproduct, and preferably in the methods as described herein.

Moreover, the disclosure relates to the following specific embodiments:

1. An Escherichia coli cell genetically modified to produce at least one bioproduct of the list comprising monosaccharide, phosphorylated monosaccharide, activated monosaccharide, disaccharide, oligosaccharide or glycolipid, wherein i) the expression of an endogenous membrane protein encoding gene is reduced and/or ii) wherein the endogenous membrane protein encoding gene is mutated, preferably, the mutation results in reduced expression of the membrane protein encoding gene, and wherein the membrane protein is any one of a protein as described in Table 1.

2. Cell according to embodiment 1, wherein the membrane protein is chosen from the list comprising: COG groups COG4206, COG2067, COG4771, COG1629, COG4580, COG2885, COG3203, COG4571, COG1538, COG3248, COG0810, COG0457; an outer membrane porin, an outer membrane protease 7, a cobalamin/cobinamide outer membrane transporter, an outer membrane channel, a maltose outer membrane channel, a ferrichrome outer membrane transporter, a Ton complex subunit, a long-chain fatty acid outer membrane channel, a nucleoside-specific channel-forming protein, a ferric enterobactin outer membrane transporter, a putative TonB-dependent outer membrane receptor, an outer membrane protein, a phage receptor.

3. Cell according to any one of embodiments 1 or 2, wherein the membrane protein is chosen from the list comprising: OmpA (SEQ ID NO: 2), OmpC (SEQ ID NO: 4), OmpF (SEQ ID NO: 6), OmpT (SEQ ID NO: 8), BtuB (SEQ ID NO: 10), TolC (SEQ ID NO: 12), LamB (SEQ ID NO: 14), FhuA (SEQ ID NO: 16), TonB (SEQ ID NO: 18), FadL (SEQ ID NO: 20), Tsx (SEQ ID NO: 22), FepA (SEQ ID NO: 24), YncD (SEQ ID NO: 26), PhoE (SEQ ID NO: 28), and NfrA (SEQ ID NO: 30), a functional homolog of any one of SEQ ID NOs: 2, 4, 6, 8, 10, 12, 14, 16, 18, 20, 22, 24, 26, 28, 30, or a membrane protein having at least 70% sequence identity to the full length amino acid sequence of any one of SEQ ID NOs: 2, 4, 6, 8, 10, 12, 14, 16, 18, 20, 22, 24, 26, 28, 30.

4. Cell according to any one of embodiments 1, 2 or 3, wherein the reduced expression of the membrane protein encoding gene and/or mutation of the membrane protein encoding gene confers bacteriophage resistance and wherein the bacteriophage is selected from the bacteriophage families listed in table 2.

5. Cell according to any one of the previous embodiments, wherein the reduced expression of the membrane protein encoding gene and/or mutation of the membrane protein encoding gene confers unaffected and/or enhanced i) bioproduct production, ii) productivity, iii) biomass production, and/or iv) cell growth.

6. Cell according to any one of the previous embodiments, wherein the mutation and/or reduced expression comprises reducing and/or abolishing the bacteriophage binding capacity of the membrane protein.

7. Cell according to any one of the previous embodiments, wherein the E. coli cell is transformed with at least one heterologous gene to produce at least any one of a sialic acid pathway or sialylation pathway, or fucosylation pathway or galactosylation pathway or N-acetylglucosamine carbohydrate pathway, preferably the cell is transformed by introduction of a heterologous gene, genetic cassette or set of genes as described in the art.

8. Cell according to any one of the previous embodiments, wherein the mutation and/or reduced expression of the endogenous membrane protein comprises any one or more of:
i) mutating the transcription unit of the membrane protein encoding gene;
ii) mutating the endogenous/homologous promoter of the membrane protein encoding gene;
iii) mutating the ribosome binding site of the membrane protein encoding gene;
iv) mutating an UTR of the membrane protein encoding gene; and/or
v) mutating the transcription terminator.

9. Cell according to any one of the previous embodiments, wherein the mutation of the membrane protein encoding gene comprises rendering the membrane protein shorter, longer and/or completely knocks out the membrane protein.

10. Cell according to any one of embodiments 1 to 9, wherein the mutation of the membrane protein encoding gene is an in-frame mutation of the membrane protein encoding gene.

11. Cell according to embodiment 10, wherein the in-frame mutation is an insertion of at least 2 amino acids into the encoded membrane protein's amino acid sequence, preferably wherein the mutation comprises an insertion of more than 2 amino acids.

12. Cell according to any one of embodiments 9 to 11, wherein the mutation occurs in the tolC encoding gene, and wherein the mutation comprises an 11 amino acid duplication of the amino acid sequence VGLSFSLPIYQ (SEQ ID NO: 31).

13. Cell according to any one of the previous embodiments, wherein at least two of the membrane protein encoding genes are mutated and/or have a reduced expression.

14. Cell according to any one of the preceding embodiments, wherein the bioproduct is an oligosaccharide, preferably the oligosaccharide is selected from the group of fucosyllactoses, sialyllactoses, Lacto-N-tetraoses, difucosyllacto-N-tetraose, sialyl-lacto-N-tetraoses, lacto-N-fucopentaoses, lewis-type antigens, more preferably selected from the group comprising 2′FL, 3FL, DiFL, Lacto-N-triose, LNT, LNnT, 3′SL, 6′SL, LSTa, LSTb, LSTc, LSTd, DFLNT, lacto-N-fucopentaose I, lacto-N-fucopentaose II, lacto-N-fucopentaose III, lacto-N-fucopentaose V, lacto-N-fucopentaose VI, H1 antigen, Lewis^{a}, Lewis^{b}, sialyl Lewis^{a}, H2 antigen, Lewis^{x}, Lewis^{y}; sialyl-Lewis^{X}.

15. Cell according to any one of the embodiments 1 to 13, wherein the bioproduct is a disaccharide preferably selected from the group comprising N-acetyllactosamine, lactose; or wherein the bioproduct is a activated monosaccharide preferably selected from the group comprising GDP-fucose, UDP-glucose, UDP-galactose, UDP-N-acetylglucosamine, CMP-sialic acid; or wherein the bioproduct is a monosaccharide preferably selected from the group comprising glucosamine, mannose, xylose, N-acetylmannosamine, N-acetylneuraminic acid, N-glycolylneuraminic acid, sialic acid, N-acetylgalactosamine, galactosamine, fucose, rhamnose, glucuronic acid, gluconic acid, or wherein the bioproduct is a phosphorylated monosaccharide preferably selected from the group comprising glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate.

16. A method for conferring bacteriophage resistance in an E. coli cell, the method comprising:
providing an E. coli cell genetically modified to produce at least one bioproduct of the list comprising monosaccharide, phosphorylated monosaccharide, activated monosaccharide, disaccharide, oligosaccharide or glycolipid, and
reducing the expression of and/or mutating a membrane protein encoding gene of the E. coli cell,
wherein the membrane protein is any one of a protein as described in Table 1.

17. A method for producing at least one bioproduct of the list comprising monosaccharide, phosphorylated monosaccharide, activated monosaccharide, disaccharide, oligosaccharide or glycolipid with an E. coli cell, the method comprising:
providing an E. coli cell genetically modified to produce at least one bioproduct of the list comprising monosaccharide, phosphorylated monosaccharide, activated monosaccharide, disaccharide, oligosaccharide or glycolipid,
reducing the expression of and/or mutating a membrane protein encoding gene of the E. coli cell,
cultivating the cell in a medium under conditions permissive for the production of the desired bioproduct, and
preferably separating the bioproduct from the cultivation; wherein the membrane protein is any one of the proteins as described in Table 1.

18. A method for increasing the production of at least one bioproduct of the list comprising monosaccharide, phosphorylated monosaccharide, activated monosaccharide, disaccharide, oligosaccharide or glycolipid with an E. coli cell as compared to an E. coli cell genetically modified to produce the bioproduct(s), the method comprising:
providing an E. coli cell genetically modified to produce at least one bioproduct of the list comprising monosaccharide, phosphorylated monosaccharide, activated monosaccharide, disaccharide, oligosaccharide or glycolipid,
reducing the expression of and/or mutating a membrane protein encoding gene of the E. coli cell,
cultivating the cell in a medium under conditions permissive for the production of the desired bioproduct, and
preferably separating the bioproduct from the cultivation; wherein the membrane protein is any one of the proteins as described in Table 1.

19. Method according to any one of embodiments 16 to 18, wherein the membrane protein is chosen from the list comprising: COG groups COG4206, COG2067, COG4771, COG1629, COG4580, COG2885, COG3203, COG4571, COG1538, COG3248, COG0810, COG0457; an outer membrane porin, an outer membrane protease 7, a cobalamin/cobinamide outer membrane transporter, an outer membrane channel, a maltose outer membrane channel, a ferrichrome outer membrane transporter, a Ton complex subunit, a long-chain fatty acid outer membrane channel, a nucleoside-specific channel-forming protein, a ferric enterobactin outer membrane transporter, a putative TonB-dependent outer membrane receptor, an outer membrane protein, a phage receptor.

20. Method according to any one of embodiments 16 to 19, wherein the membrane protein is chosen from the list comprising: OmpA (SEQ ID NO: 2), OmpC (SEQ ID NO: 4), OmpF (SEQ ID NO: 6), OmpT (SEQ ID NO: 8), BtuB (SEQ ID NO: 10), TolC (SEQ ID NO: 12), LamB (SEQ ID NO: 14), FhuA (SEQ ID NO: 16), TonB (SEQ ID NO: 18), FadL (SEQ ID NO: 20), Tsx (SEQ ID NO: 22), FepA (SEQ ID NO: 24), YncD (SEQ ID NO: 26), PhoE (SEQ ID NO: 28), and NfrA (SEQ ID NO: 30), a functional homolog of any one of SEQ ID NOs: 2, 4, 6, 8, 10, 12, 14, 16, 18, 20, 22, 24, 26, 28, 30, or a membrane protein having at least 70% sequence identity to the full length amino acid sequence of any one of SEQ ID NOs: 2, 4, 6, 8, 10, 12, 14, 16, 18, 20, 22, 24, 26, 28, 30.

21. Method according to any one of embodiments 16 to 20, wherein the modified expression and/or mutation of the membrane protein encoding gene confers bacteriophage resistance and wherein the bacteriophage is selected from the bacteriophage families listed in table 2.

22. Method according to any one of embodiments 16 to 21, wherein the modified expression and/or mutation of the membrane protein encoding gene confers unaffected and/or enhanced bioproduct production.

23. Method according to any one of embodiments 16 to 22, wherein the modified expression and/or mutation comprises reducing and/or abolishing the bacteriophage binding capacity of the membrane protein.

24. Method according to any one of the embodiments 16 to 23, wherein the E. coli cell is genetically modified to produce at least one bioproduct chosen from a fucosylated, sialylated, galactosylated oligosaccharide, N-acetylglucosamine containing oligosaccharide, or sialic acid.

25. Method according to any one of the embodiments 16 to 24, wherein the modified expression of the endogenous membrane protein encoding gene is a lower or reduced expression, preferably the lower expression comprises any one or more of:
i) mutating the transcription unit of the membrane protein encoding gene;
ii) mutating the endogenous/homologous promoter of the membrane protein encoding gene;
iii) mutating the ribosome binding site of the membrane protein encoding gene;
iv) mutating an UTR of the membrane protein encoding gene; and/or
v) mutating the transcription terminator

26. Method according to any one of the embodiments 16 to 25, wherein the mutation of the membrane protein encoding gene comprises rendering the membrane protein shorter, longer or completely knocks out the membrane protein.

27. Method according to any one of embodiments 16 to 26, wherein the mutation is an in-frame mutation of the membrane protein encoding gene, preferably the in-frame mutation is an insertion of at least 2 amino acids into the encoded membrane protein's amino acid sequence, more preferably wherein the mutation comprises an insertion of more than 2 amino acids.

28. Method according to any one of embodiments 16 to 27, wherein the mutation occurs in the tolC gene of Escherichia coli or in a functional homolog of the tolC gene in an E. coli, and wherein the mutation provides resistance against the TLS family of bacteriophages, and wherein the mutation gives rise to an 11 amino acid duplication of the amino acid sequence VGLSFSLPIYQ (SEQ ID NO: 31)

29. Method according to any one of embodiments 16 to 28, wherein at least two of the membrane protein encoding genes are mutated and/or have a reduced expression.

30. Method according to any one of embodiments 16 to 29, wherein the bioproduct is an oligosaccharide, preferably the oligosaccharide is selected from the group of fucosyllactoses, sialyllactoses, Lacto-N-tetraoses, difucosyllacto-N-tetraose, sialyl-lacto-N-tetraoses, lacto-N-fucopentaoses, lewis-type antigens, more preferably, 2′FL, 3FL, DiFL, Lacto-N-triose, LNT, LNnT, 3′SL, 6′SL, LSTa, LSTb, LSTc, LSTd, DFLNT, lacto-N-fucopentaose I, lacto-N-fucopentaose II, lacto-N-fucopentaose III, lacto-N-fucopentaose V, lacto-N-fucopentaose VI, H1 antigen, Lewis^{a}, Lewis^{b}, sialyl Lewis^{a}, H2 antigen, Lewis^{x}, Lewis^{y}; sialyl-Lewis^{X}.

31. Method according to any one of embodiments 16 to 29, wherein the bioproduct is a disaccharide preferably selected from the group comprising LacNAc, lactose; or wherein the bioproduct is an activated monosaccharide preferably selected from the group comprising GDP-fucose, UDP-glucose, UDP-galactose, UDP-N-acetylglucosamine, CMP-sialic acid; or wherein the bioproduct is a monosaccharide preferably selected from the group comprising glucosamine, mannose, xylose, N-acetylmannosamine, N-acetylneuraminic acid, N-glycolylneuraminic acid, sialic acid, N-acetylgalactosamine, galactosamine, fucose, rhamnose, glucuronic acid, gluconic acid, or wherein the bioproduct is a phosphorylated monosaccharide preferably selected from the group comprising glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate.

32. Method for fermentative production of at least one bioproduct of the list comprising monosaccharide, phosphorylated monosaccharide, activated monosaccharide, disaccharide, oligosaccharide or glycolipid using genetically modified cells to produce the bioproduct(s), comprising the steps of:
providing a cell as described in any one of the embodiments 1 to 15;
cultivating the cell in a medium under conditions permissive for the production of the desired bioproduct; and
preferably separating the bioproduct from the cultivation.

33. Method according to any one of the embodiments 16 to 32, wherein the bioproduct is LNnT, wherein preferably the membrane protein is any one or more of LamB (SEQ ID NO: 14), FhuA (SEQ ID NO: 16), FadL (SEQ ID NO: 20), and NfrA (SEQ ID NO: 30), a functional homolog of any one of SEQ ID NO: 14, SEQ ID NO: 16, SEQ ID NO: 20 and SEQ ID NO: 30, or a membrane protein having at least 70% sequence identity to the full length amino acid sequence of any one of SEQ ID NOs 14, 16, 20, 30 and wherein preferably the mutation results in a knock-out phenotype of the gene.

34. Method according to any one of the embodiments 16 to 32, wherein the bioproduct is sialyllactose, preferably 6′SL, wherein preferably the membrane protein is FhuA (SEQ ID NO: 16), a functional homolog thereof or a membrane protein having at least 70% sequence identity to the full length amino acid sequence of SEQ ID NO: 16 and wherein preferably the mutation and/or reduced expression of the membrane protein encoding gene results in a knock-out phenotype of the gene.

35. Use of a cell as described in any one of the embodiments 1 to 15.

### EXAMPLES

### Example 1: Material and Methods Escherichia coli

### Media

The Luria Broth (LB) medium consisted of 1% tryptone peptone (Difco, Erembodegem, Belgium), 0.5% yeast extract (Difco) and 0.5% sodium chloride (VWR. Leuven, Belgium). The medium for the shake flasks experiments contained 2.00 g/L NH4Cl, 5.00 g/L (NH4)2SO4, 2.993 g/L KH2PO4, 7.315 g/L K2HPO4, 8.372 g/L MOPS, 0.5 g/L NaCl, 0.5 g/L MgSO4·7H2O, 14.26 g/L sucrose or another carbon source when specified in the examples, 1 ml/L vitamin solution, 100 μl/L molybdate solution, and 1 mL/L selenium solution. The medium was set to a pH of 7 with 1M KOH. Vitamin solution consisted of 3.6 g/L FeCl2·4H2O, 5 g/L CaCl2·2H2O, 1.3 g/L MnCl2·2H2O, 0.38 g/L CuCl2·2H2O, 0.5 g/L CoCl2·6H2O, 0.94 g/L ZnCl2, 0.0311 g/L H3BO4, 0.4 g/L Na2EDTA·2H2O and 1.01 g/L thiamine·HCl. The molybdate solution contained 0.967 g/L NaMoO4·2H2O. The selenium solution contained 42 g/L Seo2.

The minimal medium for fermentations contained 6.75 g/L NH4Cl, 1.25 g/L (NH4)2SO4, 2.93 g/L KH2PO4 and 7.31 g/L KH2PO4, 0.5 g/L NaCl, 0.5 g/L MgSO4.7H2O, 14.26 g/L sucrose or another carbon source as specified in the respective examples, 1 mL/L vitamin solution, 100 μL/L molybdate solution, and 1 mL/L selenium solution with the same composition as described above.

Complex medium was sterilized by autoclaving (121° C., 21′) and minimal medium by filtration (0.22 μm Sartorius). When necessary, the medium was made selective by adding an antibiotic (e.g., chloramphenicol (20 mg/L), carbenicillin (100 mg/L), spectinomycin (40 mg/L) and/or kanamycin (50 mg/L)).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007).

Plasmids were maintained in the host E. coli DH5alpha (F⁻, phi80dlacZΔM15, Δ(lacZYA-argF) U169, deoR, recA1, endA1, hsdR17(rk⁻, mk⁺), phoA, supE44, lambda⁻, thi-1, gyrA96, relA1) bought from Invitrogen.

### Strains and Mutations

Escherichia coli K12 MG1655 [λ⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain.

Transformants carrying a Red helper plasmid pKD46 were grown in 10 ml LB media with ampicillin, (100 mg/L) and L-arabinose (10 mM) at 30° C. to an OD_{600 nm} of 0.6. The cells were made electrocompetent by washing them with 50 ml of ice-cold water, a first time, and with 1 ml ice cold water, a second time. Then, the cells were resuspended in 50 μl of ice-cold water. Electroporation was done with 50 μl of cells and 10-100 ng of linear double-stranded-DNA product by using a Gene Pulser™ (BioRad) (600 Ω, 25 μFD, and 250 volts).

After electroporation, cells were added to 1 ml LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity.

The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, repurified from an agarose gel, and suspended in elution buffer (5 mM Tris, pH 8.0).

The selected mutants (chloramphenicol or kanamycin resistant) were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers (Fw/Rv-gene-out).

For 2′FL, 3FL and diFL production, the mutant strains derived from E. coli K12 MG1655 have knock-outs of the genes lacZ, lacY, lacA, glgC, agp, pfkA, pfkB, pgi, arcA, icR, wcaJ, pgi, ion and thyA and additionally genomic knock-ins of constitutive expression constructs containing the E. coli lacY gene, a fructose kinase gene (frk) originating from Zymomonas mobilis and a sucrose phosphorylase (SP) originating from Bifidobacterium adolescentis. These genetic modifications are also described in WO2016075243 and WO2012007481. In addition, an alpha-1,2- and/or alpha-1,3-fucosyltransferase expression plasmid is added to the strains.

For LNT and LNnT production, the strain has a genomic knock out of the lacZ gene and nagB gene and knock-ins of constitutive expression constructs containing a galactoside beta-1,3-N-acetylglucosaminyltransferase (lgtA) from Neisseria meningitidis and either an N-acetylglucosamine beta-1,3-galactosyltransferase (wbgO) from Escherichia coli O55:H7 for LNT production or an N-acetylglucosamine beta-1,4-galactosyltransferase (lgtB) from Neisseria meningitidis for LNnT production.

For 3′SL and 6′SL production the strains are described in WO18122225. The mutant strain has the following gene knock-outs: lacZ, nagABCDE, nanATEK, manXYZ. Additionally, the strain has genomic knock-ins of constitutive expression constructs containing a mutated variant of the L-glutamine-D-fructose-6-phosphate aminotransferase (glmS) from Escherichia coli, a glucosamine 6-phosphate N-acetyltransferase (GNAI) from Saccharomyces cerevisiae, an N-acetylglucosamine 2-epimerase (BoAGE) from Bacteroides ovatus, an N-acetylneuraminate synthase (NeuB) from Campylobacter jejuni, a CMP-Neu5Ac synthetase (NeuA) from Campylobacter jejuni, and either a beta-galactoside alpha-2,3-sialyltransferase from Pasteurella multocida for 3′SL production or a beta-galactoside alpha-2,6-sialyltransferase from Photobacterium damselae for 6′SL production.

All constitutive promoters and UTRs originate from the libraries described by De Mey et al. (BMC Biotechnology, 2007) and Mutalik et al. (Nat. Methods 2013, No. 10, 354-360). All genes were ordered synthetically at Twist Bioscience (twistbioscience.com) or IDT (eu.idtdna.com) and the codon usage was adapted using the tools of the supplier.

All strains are stored in cryovials at −80° C. (overnight LB culture mixed in a 1:1 ratio with 70% glycerol).

### Cultivation Conditions

A preculture of 96well microtiter plate experiments was started from a cryovial, in 150 μL LB and was incubated overnight at 37° C. on an orbital shaker at 800 rpm. This culture was used as inoculum for a 96well square microtiter plate, with 400 μL MMsf medium by diluting 400×. These final 96-well culture plates were then incubated at 37° C. on an orbital shaker at 800 rpm for 72h, or shorter, or longer. At the end of the cultivation experiment samples were taken from each well to measure sugar concentrations in the broth supernatant (extracellular sugar concentrations, after spinning down the cells), or by boiling the culture broth for 15 min at 90° C. before spinning down the cells (=whole broth measurements, average of intra- and extracellular sugar concentrations).

A preculture for the bioreactor was started from an entire 1 mL cryovial of a certain strain, inoculated in 250 mL or 500 mL of MMsf medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition was set to 37° C., and maximal stirring; pressure gas flow rates were dependent on the strain and bioreactor. The pH was controlled at 6.8 using 0.5 M H2SO4 and 20% NH40H. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10M microplate reader, Tecan, Switzerland).

### Liquid Chromatography

Standards for 2′fucosyllactose, 3-fucosyllactose, difucosyllactose, Lacto-N-tetraose, Lacto-N-neotetraose, 3′sialyllactose and 6′sialyllactose were synthetized in house. Other standards such as but not limited to lactose, sucrose, glucose, fructose were purchased from Sigma.

Carbohydrates were analyzed via an UPLC-RI (Waters, USA) method, whereby RI (Refractive Index) detects the change in the refraction index of a mobile phase when containing a sample. All sugars were separated in an isocratic flow using an Acquity UPLC BEH Amide column (Waters, USA) and a mobile phase containing 75 mL acetonitrile, 25 mL Ultrapure water and 0.25 mL triethylamine (for 2′FL, 3FL, DiFL, LNT and LNnT) or containing 70 ml acetonitrile, 26 mL 150 mM ammonium acetate and 4 mL methanol with 0.05% pyrrolidine (for 3′SL and 6′SL). The column size was 2.1×50 mm with 1.7 μm particle size. The temperature of the column was set at 50° C. (for 2′FL, 3FL, DiFL, LNT, LnnT) or 25° C. (for 3′SL and 6′SL) and the pump flow rate was 0.130 mL/min.

### Example 2: Strain Resistant to a “T1-Like” or “TLS” Bacteriophage

An E. coli MG1655 K-12 strain modified to produce 2′-fucosyllactose and difucosyllactose containing the alpha-1,2-fucosyltransferase HpFutC from Helicobacter pylori (SEQ ID NO: 36) was further mutated with two distinct mutations, both in the tolC gene.

One mutation comprised an insertion of the E. coli IS1 element 374 bp downstream of the start codon and thus completely abolished the gene function of tolC (tolC_IS1, SEQ ID NO: 34).

A second mutation comprised a 33 bp duplication of the sequence (gttggcctgagcttctcgctgccgatttatcag, bp 916 to 948 of SEQ ID NO: 32), causing a direct repeat in the tolC ORF (tolC_2, SEQ ID NO: 32). This insertion causes an in-frame 11 amino acids extension in the tolC protein sequence (V306 to Q316, SEQ ID NO: 31), which, in the wild type sequence, is partially overlapping with the beta-strand transmembrane region (M301 to S311) and extending into the periplasmic domain of the protein.

Both above E. coli mutants showed to be resistant to a phage belonging to the order Caudovirales, family Siphoviridae, genus “T1-like viruses”, related to bacteriophage TLS as described in German and Misra (2001), as no lysis of the isolated cells could be detected after overnight incubation with the phage sample (shake flask culture with fermentation medium as described in example 1), while a control strain, the original 2′FL E. coli production strain, clearly was lysed (low biomass and high phage particle density)).

Without wishing to be bound by theory, it has been hypothesized that because of the 11 amino acid duplication, as a consequence, in the 3-dimensional protein structure model, the beta-sheets in the second region of the beta-barrel domain re-align and extend the outer loop in between the two beta-strands. It has further been hypothesized that this extended outer loop has an increased flexibility and hinders bacteriophage binding.

### Example 3: Evaluation of Growth and 2′FL and DiFL Production of Wild-Type tolC Vs Mutated tolC Variants in Escherichia coli

The novel “TLS” bacteriophage resistant strains described in Example 2 were evaluated in a growth experiment according to the cultivation conditions provided in Example 1. These strains contain an alpha-1,2-fucosyltransferase enzyme (HpFutC, SEQ ID NO: 36), and are able to produce 2-fucosyllactose and difucosyllactose, but differ in the tolC gene sequence present in their genome (tolC_WT: SEQ ID NO: 11; tolC_2, SEQ ID NO: 32; tolC_IS1: SEQ ID NO: 34). Each strain was grown in multiple wells of a 96-well plate. In all figures each datapoint corresponds to data from one well. The dashed horizontal line indicates the setpoint to which all datapoints were normalized. As shown in FIG. 1, the biomass obtained is clearly lower in samples of strains containing a completely inactivated tolC gene (tolC_IS1), while for strains with wild type tolC and the tolC gene variant with the 33 bp duplication (tolC_2) the obtained amount of biomass is comparable.

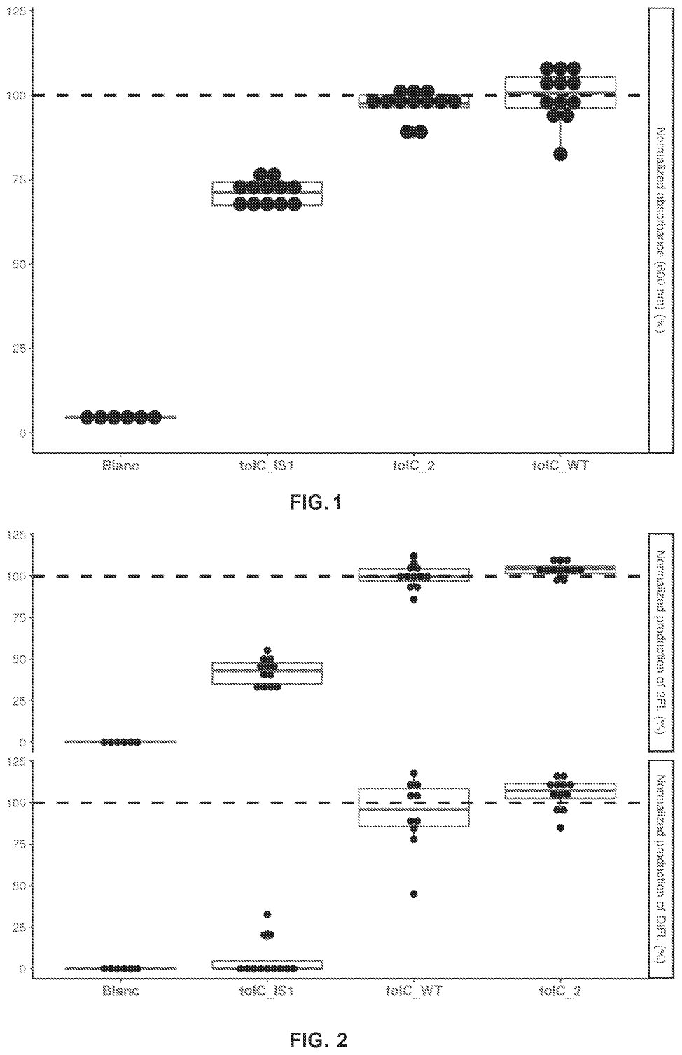

FIG. 2 shows that the production of both sugars is clearly lower in samples of strains containing a completely inactivated tolC gene (tolC_IS1), while for strains with wild type tolC and the tolC gene variant with the 33 bp duplication (tolC_2) the productivity is comparable.

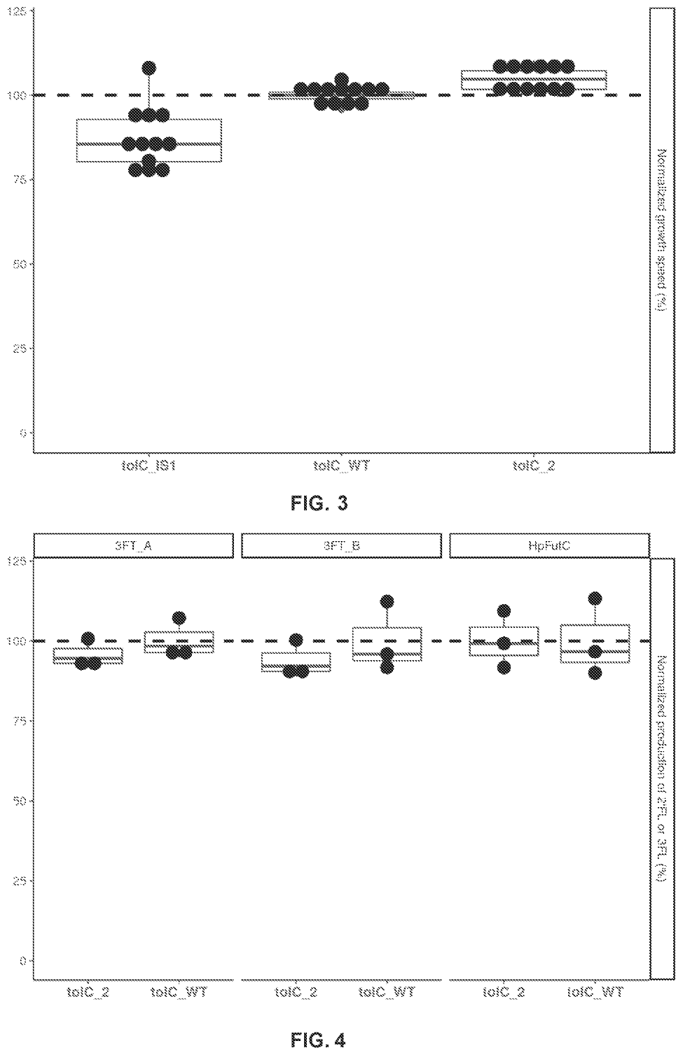

As can be seen in FIG. 3, the average growth speed is slightly lower in samples of strains containing a completely inactivated tolC gene (tolC_IS1), while for strains with wild type tolC and the tolC gene variant with the 33 bp duplication (tolC_2) this is comparable.

Altogether, these results suggest that the protein encoded by the tolC_2 gene variant is at least still partially active as a similar growth speed and 2′FL production capacity as the strain with wild type tolC is seen, while these parameters are drastically reduced in a strain carrying a completely inactivated tolC variant (tolC_IS1).

### Example 4: Evaluation of Escherichia coli Strains with a Wild Type or a Mutated tolC Gene in a Batch Fermentation for the Production of 2′Fucosyllactose

Mutant E. coli strains containing an alpha-1,2-fucosyltransferase (HpFutC, SEQ ID NO: 36) and either the wild type tolC gene sequence or the tolC variant with the 33 bp duplication conferring resistance to “TLS” bacteriophages as described in Examples 1 and 2 were evaluated in batch fermentations at bioreactor scale. The bioreactor runs were performed as described in Example 1. In these examples, sucrose was used as a carbon source. Lactose was added in the batch medium at 90 g/L as a precursor for 2′FL formation.

The batch length in time, the yield, the specific productivity and the 2′FL titer (concentration) at the end of the batch were similar for both strains. Strains with either wild type tolC or the 33 bp duplication variant of tolC (tolC_2) thus perform equally well in a biofermentation process.

### Example 5: Bacteriophage-Resistance Mutations in HMO-Producing E. coli Strains

E. coli MG1655 K-12 strains modified to produce either Lacto-N-neotetraose, 2′-fucosyllactose or 6′sialyllactose with genetic backgrounds as described in Example 1, were each further mutated with distinct mutations, all in the fhuA gene.

A first mutated strain contained an E555* point mutation introducing a premature stop codon (fhuA_E555*, SEQ ID NO: 42). A second mutated strain contained a 17 bp deletion (bp 1657 to 1673) (fhuA-fs, SEQ ID NO: 44). A third mutated strain contained an insertion of a transposon (fhuA::IS2, SEQ ID NO: 46). And a fourth mutated strain contained 75 bp in-frame deletion (bp 546 tot 620) that only partially deleted a 25 amino acid region of the protein (fhuA_2, SEQ ID NO: 48).

All of the above E. coli mutants showed to be resistant to bacteriophage T5 and T1 family (no lysis of the isolated cells after overnight incubation (shake flask culture with fermentation medium as described in example 1) while a control strain, the original oligosaccharide E. coli production strain, clearly was lysed (low biomass and high phage particle density).

All strains with and without these mutations were evaluated for growth and HMO production in both MTP growth experiments and biofermentation processes and performed equally well as or better than the control strains without these mutations on both sucrose and glycerol as carbon source.

### Example 6: Evaluation of Growth and 2′FL or 3FL Production Ofwild-Type tolC Vs Mutated tolC Variants in Escherichia coli

The wild type tolC gene of the mutant E. coli K12 MG1655 strain background, in which the fhuA gene was already replaced by the fhuA-2 (SEQ ID NO: 48) mutant gene conferring resistance to infection by bacteriophage families T5 and T1, was replaced by the tolC gene variant with the 33 bp duplication conferring TLS bacteriophage resistance (tolC_2, SEQ ID NO: 32) by the gene replacement technique as described in Example 1. Additionally, plasmids with genes coding for alpha-1,2-fucosyltransferase (HpFutC, SEQ ID NO: 36) or alpha-1,3-fucosyltransferase enzymes (3FT_A: SEQ ID NO: 38; 3FT_B: SEQ ID NO: 40) were introduced in both strains (wild type vs mutated tolC) for the production of 2′FL or 3FL, respectively. A growth experiment was performed with these strains according to the cultivation conditions provided in Example 1. Each strain was grown in multiple wells of a 96-well plate. As shown in FIG. 4, the production of both sugars is clearly similar for strains with wild type tolC vs tolC gene variant with the 33 bp duplication (tolC_2, SEQ ID NO: 32), and this for each fucosyltransferase that was tested. This tolC mutation conferring TLS bacteriophage resistance thus clearly does not impact the strain's production capabilities. This combination of both mutations in fhuA and tolC, together conferring resistance to infection against bacteriophages of the T1, T5 and TLS family, thus clearly does not impact the strain's production capabilities.

### Example 7: Evaluation of a fhuA Frame-Shift Mutation in LNnT-Producing E. coli Strains

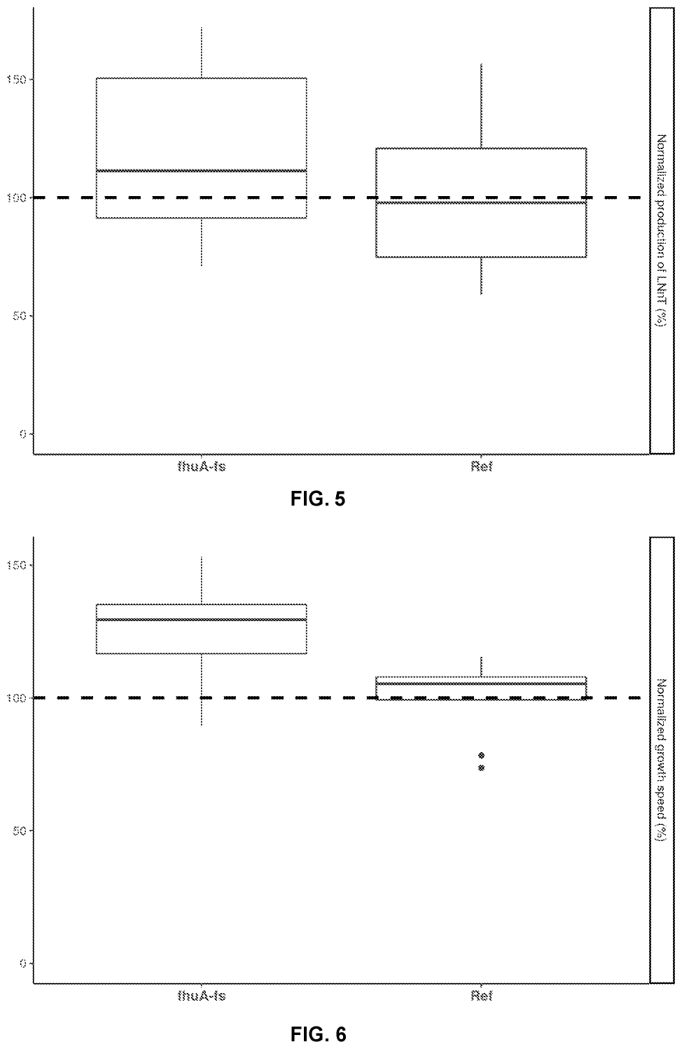

Lacto-N-neotetraose (LNnT) production strains with a genetic background as described in Example 1 with either a wild-type fhuA gene (“Ref,” SEQ ID NO: 15) or with a frame-shift mutation (17 bp deletion, bp 1657 bp 1673, “fhuA-fs,” SEQ IDNO: 44) were compared in a growth experiment according to the cultivation conditions provided in Example 1. Each strain was grown in multiple wells of a 96-well plate, and this experiment was repeated independently for 5 times. In FIGS. 5 and 6 each boxplot represents data of 15 individual datapoints in total (5 independent experiments with 3 biological replicates each). The dashed horizontal line indicates the setpoint to which all datapoints were normalized.

The production of LNnT, as shown in FIG. 5, is similar for the strain with a wild type fhuA compared to a fhuA frame-shift variant. This fhuA-fs mutation conferring resistance to the T5 and T1 family of phages thus clearly does not impact the strain's production capabilities. The growth speed, as shown in FIG. 6, is very similar for LNnT strains with a wild type fhuA or a fhuA frame-shift variant. This fhuA-fs mutation conferring resistance to the T5 and T1 family of phages thus clearly does not impact the strain's growth speed.

### Example 8: Evaluation of a fhuA::IS2 Mutation in 6′SL-Producing E. coli Strains

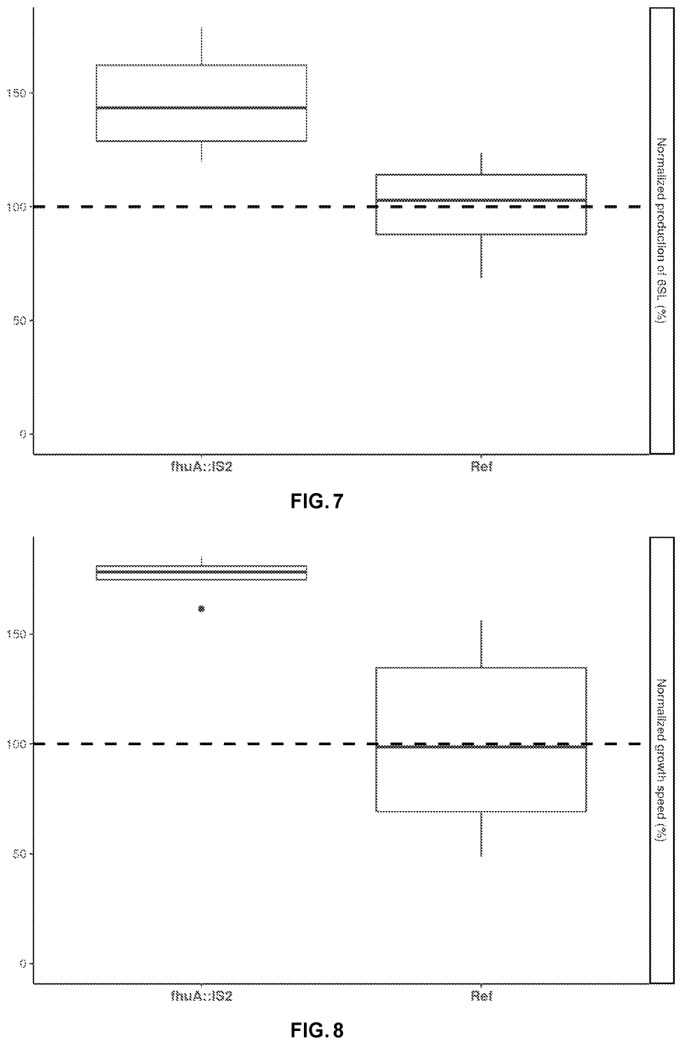

6′SL production strains with a genetic background as described in Example 1 with either a wild-type fhuA gene (“Ref,” SEQ ID NO: 15) or with a transposon insertion (“fhuA::IS2,” SEQ ID NO: 46) were compared in a growth experiment according to the cultivation conditions provided in Example 1. Each strain was grown in multiple wells of a 96-well plate, and this experiment was repeated independently for 5 times. In FIGS. 7 and 8, each boxplot represents data of 20 individual datapoints in total (5 independent experiments with 4 biological replicates each). The dashed horizontal line indicates the setpoint to which all datapoints were normalized. As can be seen in FIG. 7, the production of 6′SL is considerably higher for the strain with a fhuA::IS2 mutation compared to the strain with a wild type fhuA gene. FIG. 8 shows that the maximal growth speed is considerably higher for the strain with a fhuA::IS2 mutation compared to the strain with a wild type fhuA gene.

### Example 9: Evaluation of Knock-Outs of Various Outer Membrane Proteins in LNnT-Producing E. coli Strains

A lacto-N-neotetraose (LNnT) production strain with genetic background as described in Example 1, referred to as “REF1,” was further engineered to contain full gene knock-outs of the genes coding for the outer membrane proteins fadL (SEQ ID NO: 19), fhuA (SEQ ID NO: 15), lamB (SEQ ID NO: 13) or nfrA (SEQ ID NO: 29). Depending on the specific outer membrane protein knock-out, the strains thus gain resistance against the respective phage families as described in Table 3. These strains were compared in a growth experiment according to the cultivation conditions provided in Example 1. Each strain was grown in multiple wells of a 96-well plate.

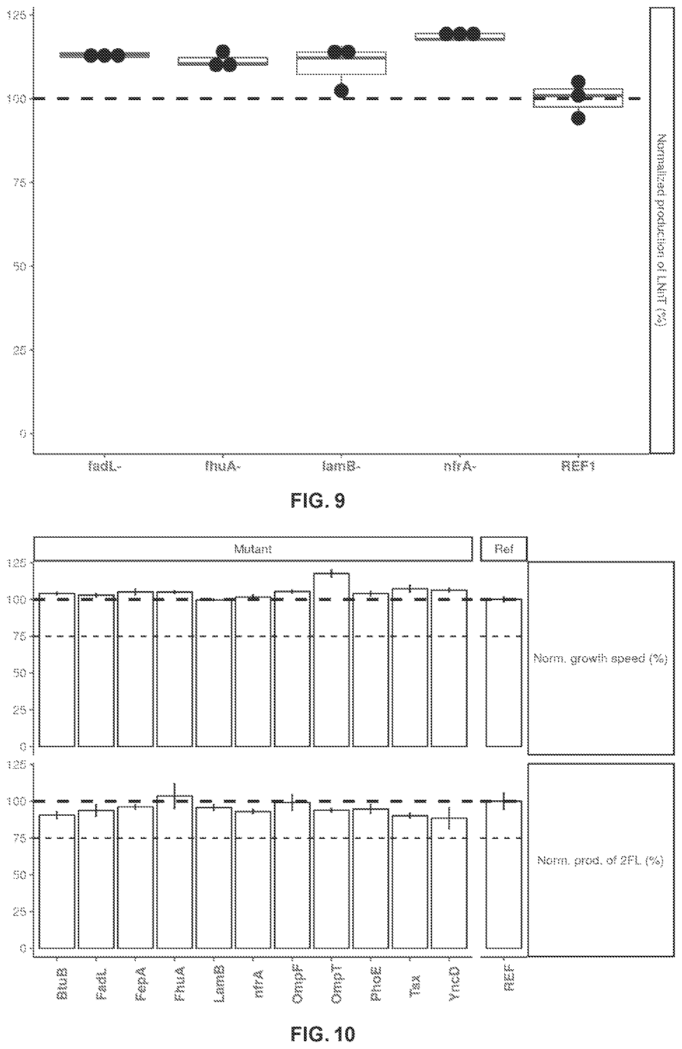

FIG. 9 shows that the production of LNnT is slightly higher for strains that are knocked out in fadL, fhuA, lamB or nfrA compared to the reference strain.

### Example 10: Evaluation of Knock-Outs of Various Outer Membrane Proteins in HMO-Producing E. coli Strains

Various strains for the production of 2′FL, 3FL, DiFL, LNT, LNnT, 3′SL and 6′SL, respectively (genetic backgrounds as described in Example 1), are engineered to contain full gene knock-outs of at least one of any one of the genes coding for the outer membrane proteins ompF (SEQ ID NO: 5), fadL (SEQ ID NO: 19), btuB (SEQ ID NO: 9), nfrA (SEQ ID NO: 29), ompA (SEQ ID NO: 1), lamB (SEQ ID NO: 13), fepA (SEQ ID NO: 23), fhuA (SEQ ID NO: 15), ompC (SEQ ID NO: 3), yncD (SEQ ID NO: 25), tsx (SEQ ID NO: 21), tolC (SEQ ID NO: 11), tonB (SEQ ID NO: 17), ompT (SEQ ID NO: 7), phoE (SEQ ID NO: 27). Depending on the specific outer membrane protein knock-out, the strains thus gain resistance against the respective phage families as described in Table 3. These strains are compared to their respective reference strains in a growth experiment according to the cultivation conditions provided in Example 1. Each strain is grown in multiple wells of a 96-well plate. The strains are evaluated on their fitness (maximal growth speed) and on their production capacity of the various HMOs as further described in Examples 18 to 22.

### Example 11: Bacteriophage Resistance in E. coli Strains Producing Phosphorylated Monosaccharides and/or Activated Monosaccharides

Mutations in membrane proteins conferring resistance to infection by certain families of bacteriophages as described herein, such as complete or partial knock-outs, in-frame or out-of-frame mutations in any one of ompF (SEQ ID NO: 5), fadL (SEQ ID NO: 19), btuB (SEQ ID NO: 9), nfrA (SEQ ID NO: 29), ompA (SEQ ID NO: 1), lamB (SEQ ID NO: 13), fepA (SEQ ID NO: 23), fhuA (SEQ ID NO: 15), ompC (SEQ ID NO: 3), yncD (SEQ ID NO: 25), tsx (SEQ ID NO: 21), tolC (SEQ ID NO: 11), tonB (SEQ ID NO: 17), ompT (SEQ ID NO: 7), phoE (SEQ ID NO: 27), are introduced in E. coli strains producing phosphorylated monosaccharides and/or activated monosaccharides. Examples of phosphorylated monosaccharides include but are not limited to glucose-1-phosphate, glucose-6-phosphate, glucose-1,6-bisophosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some but not all of these phosphorylated monosaccharides are precursors or intermediates for the production of activated monosaccharide. Examples of activated monosaccharides include but are not limited to GDP-fucose, UDP-glucose, UDP-galactose and UDP-N-acetylglucosamine. These phosphorylated monosaccharides and/or activated monosaccharides can be produced in higher amounts than naturally occurring in E. coli e.g., by introducing some of the genetic modifications as described in Example 1. An E. coli strain with active expression units of the sucrose phosphorylase and fructokinase genes (BaSP SEQ ID NO: 54, ZmFrk SEQ ID NO: 53) is able to grow on sucrose as a carbon source and can produce high(er) amounts of glucose-1P, as described in WO2012/007481. Such a strain additionally containing a knock-out of the genes pgi, pfkA and pfkB accumulate fructose-6-phosphate in the medium when grown on sucrose. Alternatively, by knocking out genes coding for (a) phosphatase(s) (agp), glucose 6-phosphate-1-dehydrogenase (zwf), phosphoglucose isomerase (pgi), glucose-1-phosphate adenylyltransferase (glgC), phosphoglucomutase (pgm) a mutant is constructed that accumulates glucose-6-phosphate.

Alternatively, the strain containing a sucrose phosphorylase and fructokinase with an additional overexpression of the wild type or variant protein of the L-glutamine-D-fructose-6-phosphate aminotransferase (glmS) from E. coli (SEQ ID NO: 57) can produce higher amounts of glucosamine-6P, glucosamine-1P and/or UDP-N-acetylglucosamine. Alternatively, by knocking out the E. coli gene wcaJ coding for the undecaprenyl-phosphate glucose phosphotransferase will have an increased pool of GDP-fucose. An increased pool of UDP-glucose and/or UDP-galactose could be achieved by overexpressing the E. coli enzymes glucose-1-phosphate uridyltransferase (galU) and/or UDP-galactose-4-epimerase (galE). Alternatively, by overexpressing genes coding for galactokinase (galK) and galactose-1-phosphate uridylyltransferase (for example, originating from Bifidobacterium bifidum) the formation of UDP-galactose is enhanced by additionally knocking out genes coding for (a) phosphatase(s) (agp), UDP-glucose, galactose-1P uridylyltransferase (galT), UDP-glucose-4-epimerase (galE) a mutant is constructed that accumulates galactose-1-phosphate.

Another example of an activated monosaccharide is CMP-sialic acid that is not naturally produced by E. coli. Production of CMP-sialic acid can e.g., be achieved by introducing genetic modifications as described in Example 1 for the 3′SL or 6′SL background strain (but without the necessity for a gene coding for a sialyltransferase enzyme).

Such strains can be used in a biofermentation process to produce these phosphorylated monosaccharides or activated monosaccharides in which the strains are grown on e.g., one or more of the following carbon sources: sucrose, glucose, glycerol, fructose, lactose, arabinose, maltotriose, sorbitol, xylose, rhamnose and mannose. Such strains additionally containing resistance mutations against one or more families of bacteriophages will have a serious advantage in industrial-scale fermentations as they will be less prone to bacteriophage infections.

### Example 12: Bacteriophage Resistance in E. coli Strains Producing Monosaccharides

Mutations in outer membrane proteins conferring resistance to infection by certain families of bacteriophages as described herein, such as complete or partial knock-outs, in-frame or out-of-frame mutations in ompF (SEQ ID NO: 5), fadL (SEQ ID NO: 19), btuB (SEQ ID NO: 9), nfrA (SEQ ID NO: 29), ompA (SEQ ID NO: 1), lamB (SEQ ID NO: 13), fepA (SEQ ID NO: 23), fhuA (SEQ ID NO: 15), ompC (SEQ ID NO: 3), yncD (SEQ ID NO: 25), tsx (SEQ ID NO: 21), tolC (SEQ ID NO: 11), tonB (SEQ ID NO: 17), ompT (SEQ ID NO: 7), phoE (SEQ ID NO: 27), can be introduced in E. coli production strains for monosaccharides. An example of such a monosaccharide is L-fucose. An E. coli fucose production strain can be created e.g., by starting from a strain that is able to produce 2′FL as described in Example 1 and by additionally knocking out the E. coli genes fucK and fucI (coding for an L-fucose isomerase and an L-fuculokinase) to avoid fucose degradation, and by expressing an 1,2-alpha-L-fucosidase (e.g., afcA from Bifidobacterium bifidum (GenBank accession no.: AY303700)) to degrade 2′FL into fucose and lactose. Such a strain can be used in a biofermentation process to produce L-fucose in which the strain is grown on sucrose, glucose or glycerol and in the presence of catalytic amounts of lactose as an acceptor substrate for the alpha-1,2-fucosyltransferase. Such a strain additionally containing resistance mutations against one or more families of bacteriophages will have a serious advantage in industrial-scale fermentations as it will be less prone to bacteriophage infections.

### Example 13: Bacteriophage Resistance in E. coli Strain Producing Disaccharides

Mutations in outer membrane proteins conferring resistance to infection by certain families of bacteriophages as described herein, such as complete or partial knock-outs, in-frame or out-of-frame mutations in ompF (SEQ ID NO: 5), fadL (SEQ ID NO: 19), btuB (SEQ ID NO: 9), nfrA (SEQ ID NO: 29), ompA (SEQ ID NO: 1), lamB (SEQ ID NO: 13), fepA (SEQ ID NO: 23), fhuA (SEQ ID NO: 15), ompC (SEQ ID NO: 3), yncD (SEQ ID NO: 25), tsx (SEQ ID NO: 21), tolC (SEQ ID NO: 11), tonB (SEQ ID NO: 17), ompT (SEQ ID NO: 7), phoE (SEQ ID NO: 27), can be introduced in E. coli strains aimed at producing disaccharides. An example of such a disaccharide is e.g., lactose (galactose-beta,1,4-glucose). An E. coli lactose production strain can be created e.g., by introducing in wild type E. coli at least one recombinant nucleic acid sequence encoding for a protein having a beta-1,4-galactosyltransferase activity and being able to transfer galactose on a free glucose monosaccharide to intracellularly generate lactose as e.g., described in WO2015150328. As such the sucrose is taken up or internalized into the host cell via a sucrose permease. Within the bacterial host cell, sucrose is degraded by invertase to fructose and glucose. The fructose is phosphorylated by fructokinase (e.g., frk from Zymomonas mobilis (SEQ ID NO: 53)) to fructose-6-phosphate, which can then be further converted to UDP-galactose by the endogenous E. coli enzymes phosphohexose isomerase (pgi), phosphoglucomutase (pgm), glucose-1-phosphate uridylyltransferase (galU) and UDP-galactose-4-epimerase (galE). A beta-1,4-galactosyltransferase (e.g., lgtB from Neisseria meningitidis, SEQ ID NO: 52) then catalyzes the reaction UDP−galactose+glucose=>UDP+lactose.

Preferably, the strain is further modified to not express the E. coli lacZ enzyme, a beta-galactosidase that would otherwise degrade lactose.

Such a strain can be used in a biofermentation process to produce lactose in which the strain is grown on sucrose as the sole carbon source. Such a strain additionally containing resistance mutations against one or more families of bacteriophages will have a serious advantage in industrial-scale fermentations as it will be less prone to bacteriophage infections.

### Example 14: Bacteriophage Resistance in E. coli Strains Producing Oligosaccharides and Grown on Carbon Sources Other than Sucrose

Mutations in outer membrane proteins conferring resistance to infection by certain families of bacteriophages as described herein, such as complete or partial knock-outs, in-frame or out-of-frame mutations in ompF (SEQ ID NO: 5), fadL (SEQ ID NO: 19), btuB (SEQ ID NO: 9), nfrA (SEQ ID NO: 29), ompA (SEQ ID NO: 1), IamB (SEQ ID NO: 13), fepA (SEQ ID NO: 23), fhuA (SEQ ID NO: 15), ompC (SEQ ID NO: 3), yncD (SEQ ID NO: 25), tsx (SEQ ID NO: 21), tolC (SEQ ID NO: 11), tonB (SEQ ID NO: 17), ompT (SEQ ID NO: 7), phoE (SEQ ID NO: 27), can be introduced in E. coli strains aimed at producing non-native or increased amounts of native saccharides, monosaccharides, phosphorylated monosaccharides, activated monosaccharides or oligosaccharides, such as, for example, human milk oligosaccharides including but not limited to 2′FL, 3FL, DiFL, LNT, LNnT, 3′SL or 6′SL. Such E. coli HMO production strains can be created e.g., by introducing one or multiple genetic modifications as described in example 1. All such strains can originate from any E. coli strain and preferably have a genomic knock out of the lacZ gene to avoid lactose degradation.

For example, for 2′FL, 3FL and diFL production, such mutant strains are further modified to contain an alpha-1,2- and/or alpha-1,3-fucosyltransferase expression construct, on a plasmid or inserted into the genome.

Another example, for LNT and LNnT production, the lacZ knock-out strain can be further modified to contain a galactoside beta-1,3-N-acetylglucosaminyltransferase (e.g., lgtA from Neisseria meningitidis, SEQ ID NO: 50) expression construct and either an N-acetylglucosamine beta-1,3-galactosyltransferase (e.g., wbgO from Escherichia coli O55:H7, SEQ ID NO: 51) for LNT production or an N-acetylglucosamine beta-1,4-galactosyltransferase (e.g., lgtB from Neisseria meningitidis, SEQ ID NO: 52) for LNnT production.

Another example, for 3′SL and 6′SL production, the lacZ knock-out strain can be further modified to contain a glucosamine 6-phosphate N-acetyltransferase (e.g., GNAI from Saccharomyces cerevisiae, SEQ ID NO: 58), an N-acetylglucosamine 2-epimerase (e.g., BoAGE from Bacteroides ovatus, SEQ ID NO: 59), an N-acetylneuraminate synthase (e.g., NeuB from Campylobacter jejuni, SEQ ID NO: 60), a CMP-Neu5Ac synthetase (e.g., NeuA from Campylobacter jejuni, SEQ ID NO: 61), and either a beta-galactoside alpha-2,3-sialyltransferase for 3′SL production (e.g., SEQ ID NO: 55) or a beta-galactoside alpha-2,6-sialyltransferase for 6′SL production (e.g., SEQ ID NO: 56).

These strains as exemplified above can further contain additional modifications to improve their productivity. Such strains can then be used in biofermentation processes to produce the desired oligosaccharide, after which the oligosaccharide is preferably purified from the broth. Such a biofermentation process needs lactose in the medium as an acceptor substrate and can be performed with any carbon source that E. coli is able to metabolize. Examples of such carbon sources include but are not limited to glucose, arabinose, maltotriose, glycerol, sorbitol, xylose, rhamnose and mannose, or any combination of two or more of these carbon sources. These strains additionally containing resistance mutations against one or more families of bacteriophages, as listed above, will have a serious advantage in industrial-scale fermentations as they will be less prone to bacteriophage infections.

### Example 15: Combinations of Mutations Conferring Resistance Against Bacteriophage Infection in E. coli Strains

Mutations in outer membrane proteins conferring resistance to infection by certain families of bacteriophages as described herein, such as complete or partial knock-outs, in-frame or out-of-frame mutations in ompF (SEQ ID NO: 5), fadL (SEQ ID NO: 19), btuB (SEQ ID NO: 9), nfrA (SEQ ID NO: 29), ompA (SEQ ID NO: 1), lamB (SEQ ID NO: 13), fepA (SEQ ID NO: 23), fhuA (SEQ ID NO: 15), ompC (SEQ ID NO: 3), yncD (SEQ ID NO: 25), tsx (SEQ ID NO: 21), tolC (SEQ ID NO: 11), tonB (SEQ ID NO: 17), ompT (SEQ ID NO: 7), phoE (SEQ ID NO: 27), can be introduced in E. coli strains aimed at producing non-native or increased amounts of native saccharides, monosaccharides, phosphorylated monosaccharides, activated monosaccharides or oligosaccharides, such as, for example, human milk oligosaccharides including but not limited to 2′FL, 3FL, DiFL, LNT, LNnT, 3′SL or 6′SL. Strains with any bacteriophage resistance mutation will have an advantage in industrial-scale fermentations as they will be less prone to bacteriophage infections. In addition, combinations of two or more of such mutations conferring bacteriophage resistance, in the same or in different outer membrane proteins, are possible. Preferably, each mutation is selected in such a way that the combination of these individual mutations gives rise to resistance against multiple families of bacteriophages. In addition, preferably each mutation individually as well as any combination of mutations increases or does not impair the strain's production as compared to a strain with the same genetic make-up but lacking the mutation in the membrane protein encoding genes. An example of two such mutations that can be combined in an HMO production strain is e.g., a 33 bp duplication in the tolC gene (SEQ ID NO: 32), which confers resistance against bacteriophages from the TLS family and any of the described mutations in fhuA (full knock-out, SEQ ID NO: 42, 44, 46 or 48) conferring resistance against bacteriophages from the T1, T5 and φ80 family. These individual mutations and any combination thereof will increase or will not decrease the strain's productivity. Combined in a single production strain, the strain will be resistant to infection by any bacteriophage of the TLS, T1, T5 and φ80 family. Such a strain can be further modified to contain additional mutations (for example, complete or partial knock-outs) in e.g., lamB (SEQ ID NO: 13) and/or fadL (SEQ ID NO: 19) and/or nfrA (SEQ ID NO: 29). These strains will in addition to their resistance against infection by bacteriophages of the TLS, T1, T5 and φ80 family also have gained resistance against bacteriophages of family K10 and/or family I and/or family T2 and/or family N4. These strains can be used in biofermentation processes to produce any of the listed sugars and can be performed with any carbon source that E. coli is able to metabolize. Examples of such carbon sources include but are not limited to glucose, arabinose, maltotriose, glycerol, sorbitol, xylose, rhamnose and mannose, or any combination of two or more of these carbon sources.

### Example 16: Identification of Membrane Protein Families

Membrane proteins were classified based on the COG (Cluster of Orthologous Groups) numbers in the eggnog database (ncbi.nlm.nih.gov/pmc/articles/PMC6324079/; eggnog.embl.de/#/app/home). The eggNOG database is a public database of orthology relationships, gene evolutionary histories and functional annotations. Identification of the COG group can be done by using a standalone version of eggNOG-mapper (https://github.com/eggnogdb/eggnog-mapper). For each of the COG groups an HMM-model can be downloaded on the eggNOG website and can be used for HMMsearch using the HMMER package (http://hmmer.org/) to protein databases.

Identification of COG group was done by using a standalone version of eggNOG-mapper, eggNOGv4.5 of eggNOG-mapperv1 (eggnogdb.embl.de/#/app/home).

The COG group of membrane proteins, as used in the disclosure, is listed in Table 4.

TABLE 4

| Membrane | | SEQ | |
|---|---|---|---|
| protein | Membrane protein description | ID NOs | COG |
| btuB | cobalamin/cobinamide outer | 09-10 | COG4206 |
| | membrane transporter | | |
| fadL | long-chain fatty acid outer membrane | 19-20 | COG2067 |
| | channel/bacteriophage T2 receptor | | |
| fepA | ferric enterobactin outer | 23-24 | COG4771 |
| | membrane transporter | | |
| fhuA | ferrichrome outer membrane | 16-16 | COG1629 |
| | transporter/phage receptor | | |
| lamB | maltose outer membrane channel/phage | 13-14 | COG4580 |
| | lambda receptor protein | | |
| ompA | outer membrane porin A | 01-02 | COG2885 |
| ompC | outer membrane porin C | 03-04 | COG3203 |
| ompF | outer membrane porin F | 05-06 | COG3203 |
| ompT | outer membrane protease VII | 07-08 | COG4571 |
| | (outer membrane protein 3b) | | |
| PhoE | outer membrane porin, | 27-28 | COG3203 |
| | outer membrane phosphoporin | | |
| TolC | outer membrane channel | 11-12 | COG1538 |
| tsx | nucleoside-specific | 21-22 | COG3248 |
| | channel-forming protein | | |
| tonB | Ton complex subunit | 17-18 | COG0810 |
| nfrA | bacteriophage N4 receptor, | 29-30 | COG0457 |
| | outer membrane protein | | |
| yncD | putative TonB-dependent | 25-26 | COG1629 |
| | outer membrane receptor | | |

### Example 17: Bacteriophage Resistance in E. coli Strains Producing Glycolipids

Mutations in outer membrane proteins conferring resistance to infection by certain families of bacteriophages as described herein, such as complete or partial knock-outs, in-frame or out-of-frame mutations in ompF (SEQ TD NO: 5), fadL (SEQ ID NO: 19), btuB (SEQ ID NO: 9), nfrA (SEQ ID NO: 29), ompA (SEQ ID NO: 1), lamB (SEQ ID NO: 13), fepA (SEQ ID NO: 23), fhuA (SEQ ID NO: 15), ompC (SEQ TD NO: 3), yncD (SEQ ID NO: 25), tsx (SEQ ID NO: 21), tolC (SEQ ID NO: 11), tonB (SEQ ID NO: 17), ompT (SEQ TD NO: 7), phoE (SEQ ID NO: 27), can be introduced in E. coli production strains for glycolipids.

An example of such a glycolipid is e.g., a rhamnolipid containing one or two rhamnose residues (mono- or dirhamnolipid). The production of monorhamnolipids can be catalyzed by the enzymatic complex rhamnosyltransferase 1 (Rt1), encoded by the rhlAB operon of Pseudomonas aeruginosa, using dTDP-L-rhamnose and beta-hydroxydecanoic acid precursors. Overexpression in an E. coli strain of this rhlAB operon, as well as overexpression of the Pseudomonas aeruginosa rmlBDAC operon genes to increase dTDP-L-rhamnose availability, allows for monorhamnolipids production, mainly containing a C10-C10 fatty acid dimer moiety. This can be achieved in various media such as rich LB medium or minimal medium with glucose as carbon source.

Such a strain additionally containing resistance mutations against one or more families of bacteriophages will have a serious advantage in industrial-scale fermentations as it will be less prone to bacteriophage infections.

### Example 18: Evaluation of Knock-Outs of Various Outer Membrane Proteins in 2′FL or 3FL Producing E. coli Strains

A strain intended for 2′FL or 3FL production with genetic background as described in Example 1, containing the fhuA_2 (SEQ ID NO: 48) mutant gene conferring resistance to infection by bacteriophage families T5 and T1, and a tolC gene variant with the 33 bp duplication conferring TLS bacteriophage resistance (tolC_2, SEQ ID NO: 32), was further engineered to contain full gene knock-outs of the genes coding for the outer membrane proteins (OMPs) ompF (SEQ ID NO: 5), fadL (SEQ ID NO: 19), btuB (SEQ ID NO: 9), nfrA (SEQ ID NO: 29), lamB (SEQ ID NO: 13), fepA (SEQ ID NO: 23), fhuA (SEQ ID NO: 15), yncD (SEQ ID NO: 25), tsx (SEQ ID NO: 21), ompT (SEQ ID NO: 7) or phoE (SEQ ID NO: 27). Depending on the specific outer membrane protein knock-out, the mutant strains thus obtained gain resistance against the respective phage families as described in Table 3. Next, a plasmid with a gene coding for an alpha-1,2-fucosyltransferase (HpFutC, SEQ ID NO: 36) or for an alpha-1,3-fucosyltransferase (3FT_A, SEQ ID NO: 38) was added to all mutant strains for the production of 2′FL or 3FL, respectively.

A growth experiment was performed with these strains according to the cultivation conditions provided in Example 1. Each strain was grown in multiple wells of a 96-well plate. As shown in FIGS. 10 and 11, the production of 2′FL or 3FL, respectively, remained higher than 75% or was almost identical compared to a reference 2′- or 3-fucosyllactose production strain lacking the additional outer membrane protein knock-out (Ref strain). Also, all tested outer membrane protein gene deletions had no or only a moderate impact on the growth of the mutant strains, reaching growth speed levels higher than 75% up till 100% of the growth speed of the reference strain. These additional OMP knock-outs together with both mutations in fhuA and tolC clearly do not impact the strain's production capabilities.

### Example 19: Evaluation of Knock-Outs of Various Outer Membrane Proteins in DiFL-Producing E. coli Strains

In a next step, another experiment was set-up with a strain intended for DiFL production with genetic background as described in Example 1, containing the fhuA_2 (SEQ ID NO: 48) mutant gene and the tolC gene variant with the 33 bp duplication (tolC_2, SEQ ID NO: 32). This strain was further engineered to contain full gene knock-outs of the genes coding for the outer membrane proteins (OMPs) ompF (SEQ ID NO: 5), fadL (SEQ ID NO: 19), btuB (SEQ ID NO: 9), nfrA (SEQ ID NO: 29), fepA (SEQ ID NO: 23), yncD (SEQ ID NO: 25), tsx (SEQ ID NO: 21), ompT (SEQ ID NO: 7) or phoE (SEQ ID NO: 27). Depending on the specific outer membrane protein knock-out, the mutant strains gain resistance against the respective phage families as described in Table 3. Next, a plasmid with a gene coding for an alpha-1,2-fucosyltransferase (HpFutC, SEQ ID NO: 36) and a plasmid with an alpha-1,3-fucosyltransferase (3FT_A, SEQ ID NO: 38) encoding gene were introduced to all mutant strains for the production of DiFL.

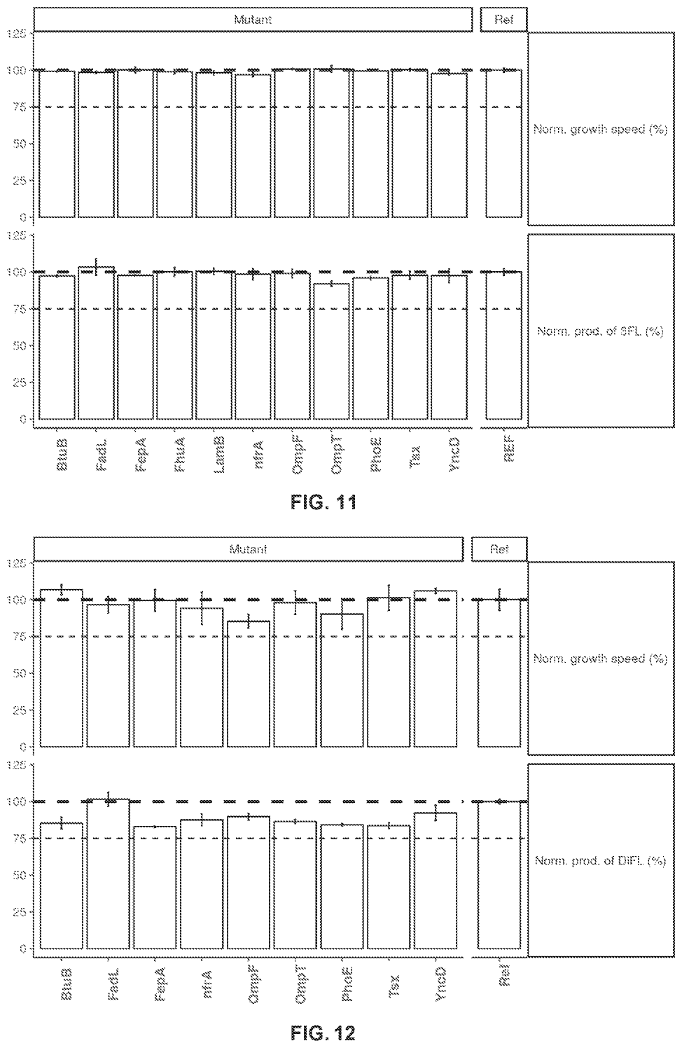

A growth experiment was performed with these strains according to the cultivation conditions provided in Example 1. Each strain was grown in multiple wells of a 96-well plate. As shown in FIG. 12, the production of DiFL remained higher than 75% or was almost identical compared to a reference strain lacking the additional outer membrane protein knock-out. Also, all tested outer membrane protein gene deletions had no or only a moderate impact on the growth of the mutant strains, reaching growth speed levels higher than 75% up till 100% of the growth speed of the reference strain. These additional OMP knock-outs together with both mutations in fhuA and tolC clearly do not impact the strain's production capabilities.

### Example 20: Evaluation of Knock-Outs of Various Outer Membrane Proteins in 6′SL or 3′SL Producing E. coli Strains

A strain intended for 6′SL or 3′SL production with genetic background as described in Example 1 was further engineered to contain full gene knock-outs of the genes coding for the outer membrane proteins (OMPs) ompF (SEQ ID NO: 5), fadL (SEQ ID NO: 19), btuB (SEQ ID NO: 9), nfrA (SEQ ID NO: 29), lamB (SEQ ID NO: 13), fepA (SEQ ID NO: 23), fhuA (SEQ ID NO: 15), yncD (SEQ ID NO: 25), tsx (SEQ ID NO: 21), ompT (SEQ ID NO: 7), phoE (SEQ ID NO: 27) or tonB (SEQ ID NO: 17). Depending on the specific outer membrane protein knock-out, the strains thus gain resistance against the respective phage families as described in Table 3. Next, a plasmid with a gene coding for an alpha-2,6-sialyltransferase (PdbST, SEQ ID NO: 56) or an alpha-2,3-sialyltransferase (PmultST3, SEQ ID NO: 55) was added to all mutant strains for the production of 6′SL or 3′SL, respectively.

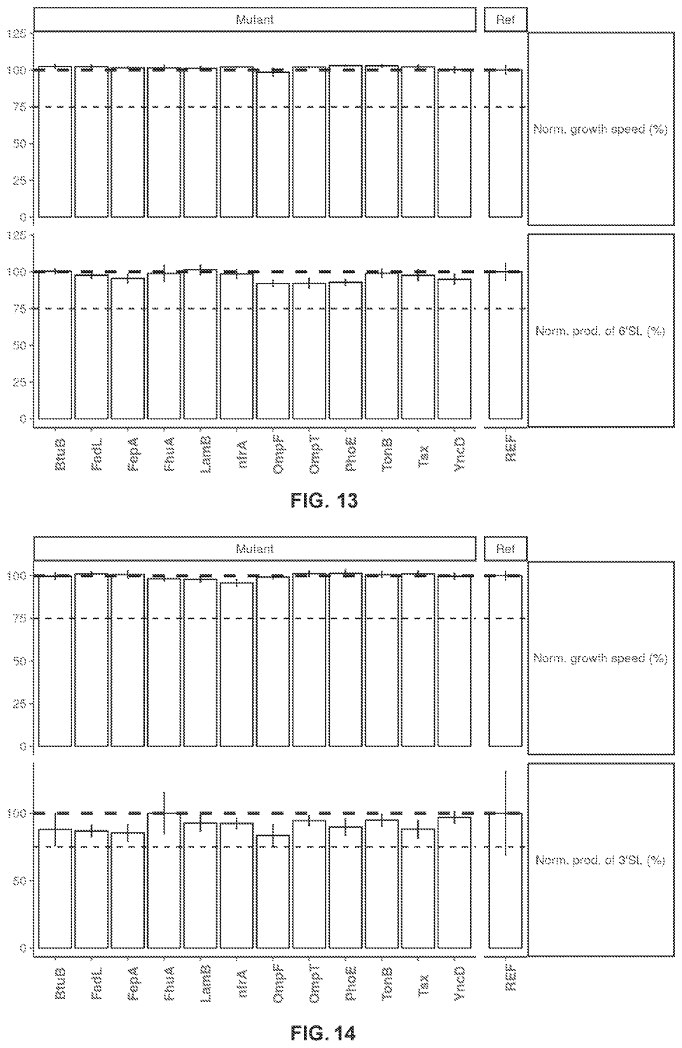

A growth experiment was performed with these strains according to the cultivation conditions provided in Example 1. Each strain was grown in multiple wells of a 96-well plate. As shown in FIGS. 13 and 14, the production of 6′SL or 3′SL, respectively, remained higher than 75% or was almost identical compared to a reference 6′- or 3′-sialyllactose production strain lacking the additional outer membrane protein knock-out. Also, all tested outer membrane protein gene deletions had no or only a moderate impact on the growth of the mutant strains, reaching growth speed levels higher than 75% up till 100% of the growth speed of the reference strain. These OMP knock-outs clearly do not impact the strain's production capabilities.

### Example 21: Evaluation of Knock-Outs of Various Outer Membrane Proteins in LNnT-Producing E. coli Strains

Additionally to the experiment described in Example 9, a mutant strain producing lacto-N-neotetraose (LNnT) and its intermediate compound lacto-N-triose (LN3) with genetic background as described in Example 1 was further engineered to contain full gene knock-outs of the genes coding for the outer membrane proteins (OMPs) ompF (SEQ ID NO: 5), btuB (SEQ ID NO: 9), fepA (SEQ ID NO: 23), yncD (SEQ ID NO: 25), tsx (SEQ ID NO: 21), ompT (SEQ ID NO: 7) or phoE (SEQ ID NO: 27). Depending on the specific outer membrane protein knock-out, the strains gain resistance against the respective phage families as described in Table 3.

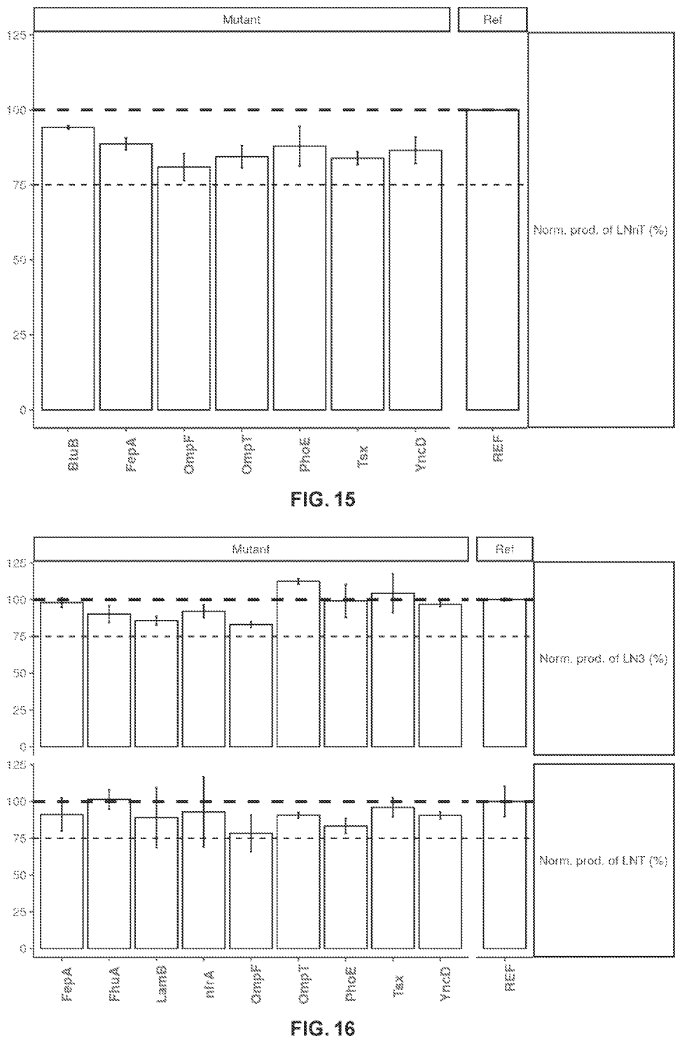

A growth experiment was performed with these strains according to the cultivation conditions provided in Example 1. Each strain was grown in multiple wells of a 96-well plate. FIG. 15 shows that the production of LNnT remained higher than 75% for all tested mutant strains as compared to the reference strain. As such, these new OMP knock-outs do not impact the strain's production capabilities. Due to the high conversion rate of LN3 to LNnT, the LN3 levels could not be measured in this experiment.

### Example 22: Evaluation of Knock-Outs of Various Outer Membrane Proteins in LNT-Producing E. coli Strains

In a next experiment, a mutant strain producing lacto-N-tetraose (LNT) and its intermediate compound lacto-N-triose (LN3) with genetic background as described in Example 1 was further engineered to contain full gene knock-outs of the genes coding for the outer membrane proteins (OMPs) ompF (SEQ ID NO: 5), nfrA (SEQ ID NO: 29), lamB (SEQ ID NO: 13), fepA (SEQ ID NO: 23), fhuA (SEQ ID NO: 15), yncD (SEQ ID NO: 25), tsx (SEQ ID NO: 21), ompT (SEQ ID NO: 7) or phoE (SEQ ID NO: 27). Depending on the specific outer membrane protein knock-out, the strains gain resistance against the respective phage families as described in Table 3.

A growth experiment was performed with these strains according to the cultivation conditions provided in Example 1. Each strain was grown in multiple wells of a 96-well plate. FIG. 16 shows that the production of the intermediate LN3 compound as well as the final LNT product remained higher than 75% for all tested mutant strains as compared to the reference strain. The mutant strain having a knock-out in ompT even showed higher LN3 production with limited effect on LNT production as compared to the reference strain with a similar genetic make-up lacking the ompT knock-out. As such, these new OMP knock-outs do not impact the strain's production capabilities.

1.-35. (canceled)

36. An Escherichia coli cell genetically modified to produce at least one bioproduct selected from the group consisting of monosaccharide, phosphorylated monosaccharide, activated monosaccharide, disaccharide, oligosaccharide, glycolipid, and any combination thereof, said cell comprising an endogenous membrane protein wherein
i) expression of the endogenous membrane protein encoding gene is reduced and/or
ii) the endogenous membrane protein encoding gene is mutated, optionally wherein the mutation results in reduced expression of the membrane protein encoding gene, and wherein the membrane protein is any one protein described in Table 1.

37. The cell of claim 36, wherein the membrane protein is selected from the group consisting of COG groups COG4206, COG2067, COG4771, COG1629, COG4580, COG2885, COG3203, COG4571, COG1538, COG3248, COG0810, COG0457; an outer membrane porin, an outer membrane protease 7, a cobalamin/cobinamide outer membrane transporter, an outer membrane channel, a maltose outer membrane channel, a ferrichrome outer membrane transporter, a Ton complex subunit, a long-chain fatty acid outer membrane channel, a nucleoside-specific channel-forming protein, a ferric enterobactin outer membrane transporter, a putative TonB-dependent outer membrane receptor, an outer membrane protein, and a phage receptor.

38. The cell of claim 36, wherein the membrane protein is selected from the group consisting of OmpA (SEQ ID NO: 2), OmpC (SEQ ID NO: 4), OmpF (SEQ ID NO: 6), OmpT (SEQ ID NO: 8), BtuB (SEQ ID NO: 10), TolC (SEQ ID NO: 12), LamB (SEQ ID NO: 14), FhuA (SEQ ID NO: 16), TonB (SEQ ID NO: 18), FadL (SEQ ID NO: 20), Tsx (SEQ ID NO: 22), FepA (SEQ ID NO: 24), YncD (SEQ ID NO: 26), PhoE (SEQ ID NO: 28), and NfrA (SEQ ID NO: 30), a functional homolog of any one of SEQ ID NOs: 2, 4, 6, 8, 10, 12, 14, 16, 18, 20, 22, 24, 26, 28, and 30, or a membrane protein having at least 70% sequence identity to the full length amino acid sequence of any one of SEQ ID NOs: 2, 4, 6, 8, 10, 12, 14, 16, 18, 20, 22, 24, 26, 28, and 30.

39. The cell of claim 36, wherein the reduced expression of the membrane protein encoding gene and/or mutation of the membrane protein encoding gene confers bacteriophage resistance and wherein the bacteriophage is selected from the bacteriophage families grouped in Table 2.

40. The cell of claim 36, wherein the reduced expression of the membrane protein encoding gene and/or mutation of the membrane protein encoding gene confers unaffected and/or enhanced i) bioproduct production, ii) productivity, iii) biomass production, and/or iv) cell growth.

41. The cell of claim 36, wherein the mutation and/or reduced expression comprises reducing and/or abolishing the bacteriophage binding capacity of the membrane protein.

42. The cell of claim 36, wherein the E. coli cell is transformed with at least one heterologous gene to produce at least any one of a sialic acid pathway, a sialylation pathway, a fucosylation pathway, a galactosylation pathway, or an N-acetylglucosamine carbohydrate pathway, and optionally wherein the cell is transformed by introduction of a heterologous gene, genetic cassette, or set of genes.

43. The cell of claim 36, wherein the mutation and/or reduced expression of the endogenous membrane protein comprises at least one of
i) mutating the transcription unit of the membrane protein encoding gene;
ii) mutating the endogenous/homologous promoter of the membrane protein encoding gene;
iii) mutating the ribosome binding site of the membrane protein encoding gene;
iv) mutating an UTR of the membrane protein encoding gene and/or
v) mutating the transcription terminator.

44. The cell of claim 36, wherein the mutation of the membrane protein encoding gene renders the membrane protein shorter, renders the membrane protein longer, and/or completely knocks out the membrane protein.

45. The cell of claim 36, wherein the mutation of the membrane protein encoding gene is an in-frame mutation of the membrane protein encoding gene.

46. The cell of claim 45, wherein the in-frame mutation is an insertion of at least two (2) amino acids into the encoded membrane protein's amino acid sequence.

47. The cell of claim 44, wherein the mutation occurs in the tolC encoding gene, and wherein the mutation comprises an eleven (11) amino acid duplication of the amino acid sequence VGLSFSLPIYQ (SEQ ID NO: 31).

48. The cell of claim 36, wherein at least two of the membrane protein encoding genes are mutated and/or have reduced expression.

49. The cell of claim 36, wherein the bioproduct is an oligosaccharide, optionally selected from the group consisting of fucosyllactoses, sialyllactoses, Lacto-N-tetraoses, difucosyllacto-N-tetraose, sialyl-lacto-N-tetraoses, lacto-N-fucopentaoses, lewis-type antigens, 2′FL, 3FL, DiFL, Lacto-N-triose, LNT, LNnT, 3′SL, 6′SL, LSTa, LSTb, LSTc, LSTd, DFLNT, lacto-N-fucopentaose I, lacto-N-fucopentaose II, lacto-N-fucopentaose III, lacto-N-fucopentaose V, lacto-N-fucopentaose VI, H1 antigen, Lewisa, Lewisb, sialyl Lewisa, H2 antigen, Lewisx, Lewisy; and sialyl-Lewisx.

50. The cell of claim 36,
wherein the bioproduct is a disaccharide optionally selected from the group consisting of N-acetyllactosamine and lactose;
wherein the bioproduct is an activated monosaccharide optionally selected from the group consisting of GDP-fucose, UDP-glucose, UDP-galactose, UDP-N-acetylglucosamine, and CMP-sialic acid;
wherein the bioproduct is a monosaccharide optionally selected from the group consisting of glucosamine, mannose, xylose, N-acetylmannosamine, N-acetylneuraminic acid, N-glycolylneuraminic acid, sialic acid, N-acetylgalactosamine, galactosamine, fucose, rhamnose, glucuronic acid, and gluconic acid, or
wherein the bioproduct is a phosphorylated monosaccharide optionally selected from the group consisting of glucose-1-phosphate, glucose-6-phosphate, glucose-1,6-bisphosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate, and fucose-1-phosphate.

51. A method for conferring bacteriophage resistance in an Escherichia coli cell, the method comprising:
providing an E. coli cell genetically modified to produce at least one bioproduct selected from the group consisting of monosaccharide, phosphorylated monosaccharide, activated monosaccharide, disaccharide, oligosaccharide, and glycolipid and any combination thereof, and
reducing the expression of and/or mutating a membrane protein encoding gene of the E. coli cell,
wherein the membrane protein is any one protein described in Table 1.

52. A method for producing at least one bioproduct selected from the group consisting of monosaccharide, phosphorylated monosaccharide, activated monosaccharide, disaccharide, oligosaccharide, and glycolipid and any combination thereof with an Escherichia coli cell, the method comprising:
providing an E. coli cell genetically modified to produce the at least one bioproduct,
reducing the expression of and/or mutating a membrane protein encoding gene of the E. coli cell,
cultivating the cell in a medium under conditions permissive for production of the bioproduct, and
optionally separating the bioproduct from the cultivation;
wherein the membrane protein is any one protein described in Table 1.

53. A method for increasing the production of at least one bioproduct selected from the group consisting of monosaccharide, phosphorylated monosaccharide, activated monosaccharide, disaccharide, oligosaccharide, and glycolipid and any combination thereof with an Escherichia coli cell in comparison to an E. coli cell genetically modified to produce the bioproduct(s), the method comprising:
providing an E. coli cell genetically modified to produce the at least one bioproduct,
reducing the expression of and/or mutating a membrane protein encoding gene of the E. coli cell,
cultivating the cell in a medium under conditions permissive for production of the bioproduct, and
optionally separating the bioproduct from the cultivation;
wherein the membrane protein is any one protein described in Table 1.

54. The method according to claim 51, wherein the membrane protein is selected from the group consisting of COG groups COG4206, COG2067, COG4771, COG1629, COG4580, COG2885, COG3203, COG4571, COG1538, COG3248, COG0810, COG0457; an outer membrane porin, an outer membrane protease 7, a cobalamin/cobinamide outer membrane transporter, an outer membrane channel, a maltose outer membrane channel, a ferrichrome outer membrane transporter, a Ton complex subunit, a long-chain fatty acid outer membrane channel, a nucleoside-specific channel-forming protein, a ferric enterobactin outer membrane transporter, a putative TonB-dependent outer membrane receptor, an outer membrane protein, and a phage receptor.

55. The method according to claim 51, wherein the membrane protein is selected from the group consisting of OmpA (SEQ ID NO: 2), OmpC (SEQ ID NO: 4), OmpF (SEQ ID NO: 6), OmpT (SEQ ID NO: 8), BtuB (SEQ ID NO: 10), TolC (SEQ ID NO: 12), LamB (SEQ ID NO: 14), FhuA (SEQ ID NO: 16), TonB (SEQ ID NO: 18), FadL (SEQ ID NO: 20), Tsx (SEQ ID NO: 22), FepA (SEQ ID NO: 24), YncD (SEQ ID NO: 26), PhoE (SEQ ID NO: 28), and NfrA (SEQ ID NO: 30), a functional homolog of any one of SEQ ID NOs: 2, 4, 6, 8, 10, 12, 14, 16, 18, 20, 22, 24, 26, 28, and 30, and a membrane protein having at least 70% sequence identity to the full length amino acid sequence of any one of SEQ ID NOs: 2, 4, 6, 8, 10, 12, 14, 16, 18, 20, 22, 24, 26, 28, and 30.

56. The method according to claim 51, wherein the modified expression and/or mutation of the membrane protein encoding gene confers bacteriophage resistance and wherein the bacteriophage is selected from the bacteriophage families grouped in Table 2.

57. The method according to claim 51, wherein the modified expression and/or mutation of the membrane protein encoding gene confers unaffected and/or enhanced bioproduct production.

58. The method according to claim 51, wherein the modified expression and/or mutation comprises reducing and/or abolishing bacteriophage binding capacity of the membrane protein.

59. The method according to claim 51, wherein the E. coli cell is genetically modified to produce at least one bioproduct selected from the group consisting of a fucosylated, sialylated, galactosylated oligosaccharide, N-acetylglucosamine containing oligosaccharide, and sialic acid.

60. The method according to claim 51, wherein the modified expression of the endogenous membrane protein encoding gene is a lower or reduced expression, optionally wherein the lower expression comprises at least one of
i) mutating the transcription unit of the membrane protein encoding gene;
ii) mutating the endogenous/homologous promoter of the membrane protein encoding gene;
iii) mutating the ribosome binding site of the membrane protein encoding gene;
iv) mutating an UTR of the membrane protein encoding gene; and/or
v) mutating the transcription terminator.

61. The method according to claim 51, wherein the mutation of the membrane protein encoding gene renders the membrane protein shorter, renders the membrane protein longer or completely knocks out the membrane protein.

62. The method according to claim 51, wherein the mutation is an in-frame mutation of the membrane protein encoding gene, optionally wherein the in-frame mutation is an insertion of at least two (2) amino acids into the encoded membrane protein's amino acid sequence.

63. The method according to claim 51, wherein the mutation occurs in the tolC gene of Escherichia coli or in a functional homolog of the tolC gene in an E. coli, and wherein the mutation provides resistance to the TLS family of bacteriophages, and wherein the mutation gives rise to an eleven (11) amino acid duplication of the amino acid sequence VGLSFSLPIYQ (SEQ ID NO: 31).

64. The method according to claim 51, wherein at least two of the membrane protein encoding genes are mutated and/or have a reduced expression.

65. The method according to claim 51, wherein the bioproduct is an oligosaccharide, optionally wherein the oligosaccharide is selected from the group consisting of fucosyllactoses, sialyllactoses, Lacto-N-tetraoses, difucosyllacto-N-tetraose, sialyl-lacto-N-tetraoses, lacto-N-fucopentaoses, Lewis-type antigens, 2′FL, 3FL, DiFL, Lacto-N-triose, LNT, LNnT, 3′SL, 6′SL, LSTa, LSTb, LSTc, LSTd, DFLNT, lacto-N-fucopentaose I, lacto-N-fucopentaose II, lacto-N-fucopentaose III, lacto-N-fucopentaose V, lacto-N-fucopentaose VI, H1 antigen, Lewisa, Lewisb, sialyl Lewisa, H2 antigen, Lewisx, Lewisy, and sialyl-Lewisx.

66. The method according to claim 51,
wherein the bioproduct is a disaccharide optionally selected from the group consisting of LacNAc, and lactose;
wherein the bioproduct is an activated monosaccharide optionally selected from the group consisting of GDP-fucose, UDP-glucose, UDP-galactose, UDP-N-acetylglucosamine, and CMP-sialic acid;
wherein the bioproduct is a monosaccharide, optionally selected from the group consisting of glucosamine, mannose, xylose, N-acetylmannosamine, N-acetylneuraminic acid, N-glycolylneuraminic acid, sialic acid, N-acetylgalactosamine, galactosamine, fucose, rhamnose, glucuronic acid, and gluconic acid, or
wherein the bioproduct is a phosphorylated monosaccharide optionally selected from the group consisting of glucose-1-phosphate, glucose-6-phosphate, glucose-1,6-bisphosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate, and fucose-1-phosphate.

67. A method for a fermentative production of at least one bioproduct selected from the group consisting of monosaccharide, phosphorylated monosaccharide, activated monosaccharide, disaccharide, oligosaccharide, and glycolipid and any combination thereof using the cell to produce the bioproduct(s), the method comprising:
using the cell of claim 36,
cultivating the cell in a medium under conditions permissive for the production of the desired bioproduct; and
optionally separating the bioproduct from the cultivation.

68. The method according to claim 51, wherein the bioproduct is LNnT, and optionally wherein the membrane protein is at least one of LamB (SEQ ID NO: 14), FhuA (SEQ ID NO: 16), FadL (SEQ ID NO: 20), and NfrA (SEQ ID NO: 30), a functional homolog of any one of SEQ ID NO: 14, SEQ ID NO: 16, SEQ ID NO: 20 and SEQ ID NO: 30, or a membrane protein having at least 70% sequence identity to the full length amino acid sequence of any one of SEQ ID NOs 14, 16, 20, 30 and wherein optionally the mutation results in a knock-out phenotype of the gene.

69. The method according to claim 51, wherein the bioproduct is sialyllactose, optionally 6′SL, optionally wherein the membrane protein is FhuA (SEQ ID NO: 16), a functional homolog thereof or a membrane protein having at least 70% sequence identity to the full length amino acid sequence of SEQ ID NO: 16 and wherein optionally the mutation and/or reduced expression of the membrane protein encoding gene results in a knock-out phenotype of the gene.


SEQUENCE LISTING (61 sequences)

SEQ ID NO: 1 - DNA, 1041 bp, Escherichia coli
atgaaaaagacagctatcgcgattgcagtggcactggctggtttcgctaccgtagcgcaggccgctccgaaagataacacctggtacactggtgctaaactgggctggtcccagtaccatgacactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggaccaacaacatcggtgacgcacacaccatcggcactcgtccggacaacggcatgctgagcctgggtgtttcctaccgtttcggtcagggcgaagcagctccagtagttgctccggctccagctccggcaccggaagtacagaccaagcacttcactctgaagtctgacgttctgttcaacttcaacaaagcaaccctgaaaccggaaggtcaggctgctctggatcagctgtacagccagctgagcaacctggatccgaaagacggttccgtagttgttctgggttacaccgaccgcatcggttctgacgcttacaaccagggtctgtccgagcgccgtgctcagtctgttgttgattacctgatctccaaaggtatcccggcagacaagatctccgcacgtggtatgggcgaatccaacccggttactggcaacacctgtgacaacgtgaaacagcgtgctgcactgatcgactgcctggctccggatcgtcgcgtagagatcgaagttaaaggtatcaaagacgttgtaactcagccgcaggcttaa

SEQ ID NO: 2 - protein, 346 aa, Escherichia coli
MKKTAIAIAVALAGFATVAQAAPKDNTWYTGAKLGWSQYHDTGFINNNGPTHENQLGAGAFGGYQVNPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGMLSLGVSYRFGQGEAAPVVAPAPAPAPEVQTKHFTLKSDVLFNFNKATLKPEGQAALDQLYSQLSNLDPKDGSVVVLGYTDRIGSDAYNQGLSERRAQSVVDYLISKGIPADKISARGMGESNPVTGNTCDNVKQRAALIDCLAPDRRVEIEVKGIKDVVTQPQA

SEQ ID NO: 3 - DNA, 1104 bp, Escherichia coli
atgaaagttaaagtactgtccctcctggtcccagctctgctggtagcaggcgcagcaaacgctgctgaagtttacaacaaagacggcaacaaattagatctgtacggtaaagtagacggcctgcactatttctctgacaacaaagatgtagatggcgaccagacctacatgcgtcttggcttcaaaggtgaaactcaggttactgaccagctgaccggttacggccagtgggaatatcagatccagggcaacagcgctgaaaacgaaaacaactcctggacccgtgtggcattcgcaggtctgaaattccaggatgtgggttctttcgactacggtcgtaactacggcgttgtttatgacgtaacttcctggaccgacgtactgccagaattcggtggtgacacctacggttctgacaacttcatgcagcagcgtggtaacggcttcgcgacctaccgtaacactgacttcttcggtctggttgacggcctgaactttgctgttcagtaccagggtaaaaacggcaacccatctggtgaaggctttactagtggcgtaactaacaacggtcgtgacgcactgcgtcaaaacggcgacggcgtcggcggttctatcacttatgattacgaaggtttcggtatcggtggtgcgatctccagctccaaacgtactgatgctcagaacaccgctgcttacatcggtaacggcgaccgtgctgaaacctacactggtggtctgaaatacgacgctaacaacatctacctggctgctcagtacacccagacctacaacgcaactcgcgtaggttccctgggttgggcgaacaaagcacagaacttcgaagctgttgctcagtaccagttcgacttcggtctgcgtccgtccctggcttacctgcagtctaaaggtaaaaacctgggtcgtggctacgacgacgaagatatcctgaaatatgttgatgttggtgctacctactacttcaacaaaaacatgtccacctacgttgactacaaaatcaacctgctggacgacaaccagttcactcgtgacgctggcatcaacactgataacatcgtagctctgggtctggtttaccagttctaa

SEQ ID NO: 4 - protein, 367 aa, Escherichia coli
MKVKVLSLLVPALLVAGAANAAEVYNKDGNKLDLYGKVDGLHYFSDNKDVDGDQTYMRLGFKGETQVTDQLTGYGQWEYQIQGNSAENENNSWTRVAFAGLKFQDVGSFDYGRNYGVVYDVTSWTDVLPEFGGDTYGSDNFMQQRGNGFATYRNTDFFGLVDGLNFAVQYQGKNGNPSGEGFTSGVTNNGRDALRQNGDGVGGSITYDYEGFGIGGAISSSKRTDAQNTAAYIGNGDRAETYTGGLKYDANNIYLAAQYTQTYNATRVGSLGWANKAQNFEAVAQYQFDFGLRPSLAYLQSKGKNLGRGYDDEDILKYVDVGATYYFNKNMSTYVDYKINLLDDNQFTRDAGINTDNIVALGLVYQF

SEQ ID NO: 5 - DNA, 1089 bp, Escherichia coli
atgatgaagcgcaatattctggcagtgatcgtccctgctctgttagtagcaggtactgcaaacgctgcagaaatctataacaaagatggcaacaaagtagatctgtacggtaaagctgttggtctgcattatttttccaagggtaacggtgaaaacagttacggtggcaatggcgacatgacctatgcccgtcttggttttaaaggggaaactcaaatcaattccgatctgaccggttatggtcagtgggaatataacttccagggtaacaactctgaaggcgctgacgctcaaactggtaacaaaacgcgtctggcattcgcgggtcttaaatacgctgacgttggttctttcgattacggccgtaactacggtgtggtttatgatgcactgggttacaccgatatgctgccagaatttggtggtgatactgcatacagcgatgacttcttcgttggtcgtgttggcggcgttgctacctatcgtaactccaacttctttggtctggttgatggcctgaacttcgctgttcagtacctgggtaaaaacgagcgtgacactgcacgccgttctaacggcgacggtgttggcggttctatcagctacgaatacgaaggctttggtatcgttggtgcttatggtgcagctgaccgtaccaacctgcaagaagctcaacctcttggcaacggtaaaaaagctgaacagtgggctactggtctgaagtacgacgcgaacaacatctacctggcagcgaactacggtgaaacccgtaacgctacgccgatcactaataaatttacaaacaccagcggcttcgccaacaaaacgcaagacgttctgttagttgcgcaataccagttcgatttcggtctgcgtccgtccatcgcttacaccaaatctaaagcgaaagacgtagaaggtatcggtgatgttgatctggtgaactactttgaagtgggcgcaacctactacttcaacaaaaacatgtccacctatgttgactacatcatcaaccagatcgattctgacaacaaactgggcgtaggttcagacgacaccgttgctgtgggtatcgtttaccagttctaa

SEQ ID NO: 6 - protein, 362 aa, Escherichia coli
MMKRNILAVIVPALLVAGTANAAEIYNKDGNKVDLYGKAVGLHYFSKGNGENSYGGNGDMTYARLGFKGETQINSDLTGYGQWEYNFQGNNSEGADAQTGNKTRLAFAGLKYADVGSFDYGRNYGVVYDALGYTDMLPEFGGDTAYSDDFFVGRVGGVATYRNSNFFGLVDGLNFAVQYLGKNERDTARRSNGDGVGGSISYEYEGFGIVGAYGAADRTNLQEAQPLGNGKKAEQWATGLKYDANNIYLAANYGETRNATPITNKFTNTSGFANKTQDVLLVAQYQFDFGLRPSIAYTKSKAKDVEGIGDVDLVNYFEVGATYYFNKNMSTYVDYIINQIDSDNKLGVGSDDTVAVGIVYQF

SEQ ID NO: 7 - DNA, 954 bp, Escherichia coli
atgcgggcgaaacttctgggaatagtcctgacaacccctattgcgatcagctcttttgcttctaccgagactttatcgtttactcctgacaacataaatgcggacattagtcttggaactctgagcggaaaaacaaaagagcgtgtttatctagccgaagaaggaggccgaaaagtcagtcaactcgactggaaattcaataacgctgcaattattaaaggtgcaattaattgggatttgatgccccagatatctatcggggctgctggctggacaactctcggcagccgaggtggcaatatggtcgatcaggactggatggattccagtaaccccggaacctggacggatgaaagtagacaccctgatacacaactcaattatgccaacgaatttgatctgaatatcaaaggctggctcctcaacgaacccaattaccgcctgggactcatggccggatatcaggaaagccgttatagctttacagccagaggtggttcctatatctacagttctgaggagggattcagagatgatatcggctccttcccgaatggagaaagagcaatcggctacaaacaacgttttaaaatgccctacattggcttgactggaagttatcgttatgaagattttgaactcggtggcacatttaaatacagcggctgggtggaatcatctgataacgatgaacactatgacccgggaaaaagaatcacttatcgcagtaaggtcaaagaccaaaattactattctgttgcagtcaatgcaggttattacgtcacacctaacgcaaaagtttatgttgaaggcgcatggaatcgggttacgaataaaaaaggtaatacttcactttatgatcacaataataacacttcagactacagcaaaaatggagcaggtatagaaaactataacttcatcactactgctggtcttaagtacacattttaa

SEQ ID NO: 8 - protein, 317 aa, Escherichia coli
MRAKLLGIVLTTPIAISSFASTETLSFTPDNINADISLGTLSGKTKERVYLAEEGGRKVSQLDWKFNNAAIIKGAINWDLMPQISIGAAGWTTLGSRGGNMVDQDWMDSSNPGTWTDESRHPDTQLNYANEFDLNIKGWLLNEPNYRLGLMAGYQESRYSFTARGGSYIYSSEEGFRDDIGSFPNGERAIGYKQRFKMPYIGLTGSYRYEDFELGGTFKYSGWVESSDNDEHYDPGKRITYRSKVKDQNYYSVAVNAGYYVTPNAKVYVEGAWNRVTNKKGNTSLYDHNNNTSDYSKNGAGIENYNFITTAGLKYTF

SEQ ID NO: 9 - DNA, 1845 bp, Escherichia coli
atgattaaaaaagcttcgctgctgacggcgtgttccgtcacggcattttccgcttgggcacaggataccagcccggatactctcgtcgttactgctaaccgttttgaacagccgcgcagcactgtgcttgcaccaaccaccgttgtgacccgtcaggatatcgaccgctggcagtcgacctcggtcaatgatgtgctgcgccgtcttccgggcgtcgatatcacccaaaacggcggttcaggtcagctctcatctatttttattcgcggtacaaatgccagtcatgtgttggtgttaattgatggcgtacgcctgaatctggcgggggtgagtggttctgccgaccttagccagttccctattgcgcttgtccagcgtgttgaatatatccgtgggccgcgctccgctgtttatggttccgatgcaataggcggggtggtgaatatcatcacgacgcgcgatgaacccggaacggaaatttcagcagggtggggaagcaatagttatcagaactatgatgtctctacgcagcaacaactgggggataagacacgggtaacgctgttgggcgattatgcccatactcatggttatgatgttgttgcctatggtaataccggaacgcaagcgcagacagataacgatggttttttaagtaaaacgctttatggcgcgctggagcataactttactgatgcctggagcggctttgtgcgcggctatggctatgataaccgtaccaattatgacgcgtattattctcccggttcaccgttgctcgatacccgtaaactctatagccaaagttgggacgccgggctgcgctataacggcgaactgattaaatcacaactcattaccagctatagccatagcaaagattacaactacgatccccattatggtcgttatgattcgtcggcgacgctcgatgagatgaagcaatacaccgtccagtgggcaaacaatgtcatcgttggtcacggtagtattggtgcgggtgtcgactggcagaaacagactacgacgccgggtacaggttatgttgaggatggatatgatcaacgtaataccggcatctatctgaccgggctgcaacaagtcggcgattttacctttgaaggcgcagcacgcagtgacgataactcacagtttggtcgtcatggaacctggcaaaccagcgccggttgggaattcatcgaaggttatcgcttcattgcttcctacgggacatcttataaggcaccaaatctggggcaactgtatggcttctacggaaatccgaatctggacccggagaaaagcaaacagtgggaaggcgcgtttgaaggcttaaccgctggggtgaactggcgtatttccggatatcgtaacgatgtcagtgacttgatcgattatgatgatcacaccctgaaatattacaacgaagggaaagcgcggattaagggcgtcgaggcgaccgccaattttgataccggaccactgacgcatactgtgagttatgattatgtcgatgcgcgcaatgcgattaccgacacgccgttgttacgccgtgctaaacagcaggtgaaataccagctcgactggcagttgtatgacttcgactggggtattacttatcagtatttaggcactcgctatgataaggattactcatcttatccttatcaaaccgttaaaatgggcggtgtgagcttgtgggatcttgcggttgcgtatccggtcacctctcacctgacagttcgtggtaaaatagccaacctgttcgacaaagattatgagacagtctatggctaccaaactgcaggacgggaatacaccttgtctggcagctacaccttctga

SEQ ID NO: 10 - protein, 614 aa, Escherichia coli
MIKKASLLTACSVTAFSAWAQDTSPDTLVVTANRFEQPRSTVLAPTTVVTRQDIDRWQSTSVNDVLRRLPGVDITQNGGSGQLSSIFIRGTNASHVLVLIDGVRLNLAGVSGSADLSQFPIALVQRVEYIRGPRSAVYGSDAIGGVVNIITTRDEPGTEISAGWGSNSYQNYDVSTQQQLGDKTRVTLLGDYAHTHGYDVVAYGNTGTQAQTDNDGFLSKTLYGALEHNFTDAWSGFVRGYGYDNRTNYDAYYSPGSPLLDTRKLYSQSWDAGLRYNGELIKSQLITSYSHSKDYNYDPHYGRYDSSATLDEMKQYTVQWANNVIVGHGSIGAGVDWQKQTTTPGTGYVEDGYDQRNTGIYLTGLQQVGDFTFEGAARSDDNSQFGRHGTWQTSAGWEFIEGYRFIASYGTSYKAPNLGQLYGFYGNPNLDPEKSKQWEGAFEGLTAGVNWRISGYRNDVSDLIDYDDHTLKYYNEGKARIKGVEATANFDTGPLTHTVSYDYVDARNAITDTPLLRRAKQQVKYQLDWQLYDFDWGITYQYLGTRYDKDYSSYPYQTVKMGGVSLWDLAVAYPVTSHLTVRGKIANLFDKDYETVYGYQTAGREYTLSGSYTF

SEQ ID NO: 11 - DNA, 1482 bp, Escherichia coli
atgaagaaattgctccccattcttatcggcctgagcctttctgggttcagttcgttgagccaggccgagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccttgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactga

SEQ ID NO: 12 - protein, 493 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 13 - DNA, 1341 bp, Escherichia coli
atgatgattactctgcgcaaacttcctctggcggttgccgtcgcagcgggcgtaatgtctgctcaggcaatggctgttgatttccacggctatgcacgttccggtattggttggacaggtagcggcggtgaacaacagtgtttccagactaccggtgctcaaagtaaataccgtcttggcaacgaatgtgaaacttatgctgaattaaaattgggtcaggaagtgtggaaagagggcgataagagcttctatttcgacactaacgtggcctattccgtcgcacaacagaatgactgggaagctaccgatccggccttccgtgaagcaaacgtgcagggtaaaaacctgatcgaatggctgccaggctccaccatctgggcaggtaagcgcttctaccaacgtcatgacgttcatatgatcgacttctactactgggatatttctggtcctggtgccggtctggaaaacatcgatgttggcttcggtaaactctctctggcagcaacccgctcctctgaagctggtggttcttcctctttcgccagcaacaatatttatgactataccaacgaaaccgcgaacgacgttttcgatgtgcgtttagcgcagatggaaatcaacccgggcggcacattagaactgggtgtcgactacggtcgtgccaacttgcgtgataactatcgtctggttgatggcgcatcgaaagacggctggttattcactgctgaacatactcagagtgtcctgaagggctttaacaagtttgttgttcagtacgctactgactcgatgacctcgcagggtaaagggctgtcgcagggttctggcgttgcatttgataacgaaaaatttgcctacaatatcaacaacaacggtcacatgctgcgtatcctcgaccacggtgcgatctccatgggcgacaactgggacatgatgtacgtgggtatgtaccaggatatcaactgggataacgacaacggcaccaagtggtggaccgtcggtattcgcccgatgtacaagtggacgccaatcatgagcaccgtgatggaaatcggctacgacaacgtcgaatcccagcgcaccggcgacaagaacaatcagtacaaaattaccctcgcacaacaatggcaggctggcgacagcatctggtcacgcccggctattcgtgtcttcgcaacctacgccaagtgggatgagaaatggggttacgactacaccggtaacgctgataacaacgcgaacttcggcaaagccgttcctgctgatttcaacggcggcagcttcggtcgtggcgacagcgacgagtggaccttcggtgcccagatggaaatctggtggtaa

SEQ ID NO: 14 - protein, 446 aa, Escherichia coli [sequence omitted]

SEQ ID NO: 15 - DNA, 2244 bp, Escherichia coli
atggcgcgttccaaaactgctcagccaaaacactcactgcgtaaaatcgcagttgtagtagccacagcggttagcggcatgtctgtttatgcacaggcagcggttgaaccgaaagaagacactatcaccgttaccgctgcacctgcgccgcaagaaagcgcatgggggcctgctgcaactattgcggcgcgacagtctgctaccggcactaaaaccgatacgccgattcaaaaagtgccacagtctatttctgttgtgaccgccgaagagatggcgctgcatcagccgaagtcggtaaaagaagcgcttagctacacgccgggtgtctctgttggtacgcgtggcgcatccaacacctatgaccacctgatcattcgcggctttgcggcagaaggccaaagccagaataactatctgaatggcctgaagttgcagggcaacttctataacgatgcggtcattgacccgtatatgctggaacgcgctgaaattatgcgtggcccggtttccgtgctttacggtaaaagcagtcctggcggcctgttgaatatggtcagcaagcgtccgaccaccgaaccgctgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtatgaagtcggcgtgaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 16 - protein, 747 aa, Escherichia coli
MARSKTAQPKHSLRKIAVVVATAVSGMSVYAQAAVEPKEDTITVTAAPAPQESAWGPAATIAARQSATGTKTDTPIQKVPQSISVVTAEEMALHQPKSVKEALSYTPGVSVGTRGASNTYDHLIIRGFAAEGQSQNNYLNGLKLQGNFYNDAVIDPYMLERAEIMRGPVSVLYGKSSPGGLLNMVSKRPTTEPLKEVQFKAGTDSLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQYEVGVKYVPEDRPIVVTGAVYNLTKTNNLMADPEGSFFSVEGGEIRARGVEIEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 17 - DNA, 720 bp, Escherichia coli
atgacccttgatttacctcgccgcttcccctggccgacgttactttcggtctgcattcatggtgctgttgtggcgggtctgctctatacctcggtacatcaggttattgaactacctgcgcctgcgcagccgatttctgtcacgatggttacgcctgctgatctcgaaccgccacaagccgttcagccgccaccggagccggtggtagagccagaaccggaacctgagccgatccccgaaccgccaaaagaagcaccggtggtcattgaaaagccgaagccgaaacctaagccaaaaccgaagccggtgaaaaaggtacaggagcagccaaaacgcgatgtcaaacccgtagagtcgcgtccggcatcaccgtttgaaaatacggcaccggcacgcctgacatcaagtacagcaacggctgcaaccagcaagccggttaccagtgtggcttcaggaccacgcgcattaagccgtaatcagccgcagtatccggcacgagcacaggcattgcgcattgaagggcaggttaaagttaaatttgacgttacgccggatggtcgcgtggataacgtacaaatcctctcagccaagcctgcgaacatgtttgagcgtgaggtgaaaaatgcgatgcgcagatggcgttatgagccgggtaagccaggcagtgggattgtggtgaatatcctgtttaaaattaacggcaccaccgaaattcagtaa

SEQ ID NO: 18 - protein, 239 aa, Escherichia coli
MTLDLPRRFPWPTLLSVCIHGAVVAGLLYTSVHQVIELPAPAQPISVTMVTPADLEPPQAVQPPPEPVVEPEPEPEPIPEPPKEAPVVIEKPKPKPKPKPKPVKKVQEQPKRDVKPVESRPASPFENTAPARLTSSTATAATSKPVTSVASGPRALSRNQPQYPARAQALRIEGQVKVKFDVTPDGRVDNVQILSAKPANMFEREVKNAMRRWRYEPGKPGSGIVVNILFKINGTTEIQ

SEQ ID NO: 19 - DNA, 1341 bp, Escherichia coli
atgagccagaaaaccctgtttacaaagtctgctctcgcagtcgcagtggcacttatctccacccaggcctggtcggcaggctttcagttaaacgaattttcttcctctggcctgggccgggcttattcaggggaaggcgcaattgccgatgatgcaggtaacgtcagccgtaaccccgcattgattactatgtttgaccgcccgacattttctgcgggtgcggtttatattgacccggatgtaaatatcagcggaacgtctccatctggtcgtagcctgaaagccgataacatcgcgcctacggcatgggttccgaacatgcactttgttgcaccgattaacgaccaatttggttggggcgcttctattacctctaactatggtctggctacagagtttaacgatacttatgcaggcggctctgtcgggggtacaaccgaccttgaaaccatgaacctgaacttaagcggtgcgtatcgcttaaataatgcatggagctttggtcttggtttcaacgccgtctacgctcgcgcgaaaattgaacgtttcgcaggcgatctggggcagttggttgctggccaaattatgcaatctcctgctggccaaactcagcaagggcaagcattggcagctaccgccaacggtattgacagtaataccaaaatcgctcatctgaacggtaaccagtggggctttggctggaacgccggaatcctgtatgaactggataaaaataaccgctatgcactgacctaccgttctgaagtgaaaattgacttcaaaggtaactacagcagcgatcttaatcgtgcgtttaataactacggtttgccaattcctaccgcgacaggtggcgcaacgcaatcgggttatctgacgctgaacctgcctgaaatgtgggaagtgtcaggttataaccgtgttgatccacagtgggcgattcactatagcctggcttacaccagctggagtcagttccagcagctgaaagcgacctcaaccagtggcgacacgctgttccagaaacatgaaggctttaaagatgcttaccgcatcgcgttgggtaccacttattactacgatgataactggaccttccgtaccggtatcgcctttgatgacagcccagttcctgcacagaatcgttctatctccattccggaccaggaccgtttctggctgagtgcaggtacgacttacgcatttaataaagatgcttcagtcgacgttggtgtttcttatatgcacggtcagagcgtgaaaattaacgaaggcccataccagttcgagtctgaaggtaaagcctggctgttcggtactaactttaactacgcgttctga

SEQ ID NO: 20 - protein, 446 aa, Escherichia coli
MSQKTLFTKSALAVAVALISTQAWSAGFQLNEFSSSGLGRAYSGEGAIADDAGNVSRNPALITMFDRPTFSAGAVYIDPDVNISGTSPSGRSLKADNIAPTAWVPNMHFVAPINDQFGWGASITSNYGLATEFNDTYAGGSVGGTTDLETMNLNLSGAYRLNNAWSFGLGFNAVYARAKIERFAGDLGQLVAGQIMQSPAGQTQQGQALAATANGIDSNTKIAHLNGNQWGFGWNAGILYELDKNNRYALTYRSEVKIDFKGNYSSDLNRAFNNYGLPIPTATGGATQSGYLTLNLPEMWEVSGYNRVDPQWAIHYSLAYTSWSQFQQLKATSTSGDTLFQKHEGFKDAYRIALGTTYYYDDNWTFRTGIAFDDSPVPAQNRSISIPDQDRFWLSAGTTYAFNKDASVDVGVSYMHGQSVKINEGPYQFESEGKAWLFGTNFNYAF

SEQ ID NO: 21 - DNA, 885 bp, Escherichia coli
atgaaaaaaacattactggcagccggtgcggtactggcgctctcttcgtcttttactgtcaacgcagctgaaaacgacaaaccgcagtatctttccgactggtggcaccagagcgttaacgttgtcggaagctatcacacccgtttcggaccgcagatccgcaacgatacctaccttgagtacgaagcattcgctaaaaaagactggttcgacttctatggttatgcggatgcgccggtattcttcggcggtaactccgatgctaaaggtatctggaaccacggttctccgctgtttatggaaatcgaaccacgtttctccatcgacaagctgaccaatactgaccttagcttcggtccgttcaaagagtggtacttcgcgaacaactacatttacgacatgggtcgtaataaagatggtcgccagagcacctggtacatgggtctgggtaccgatatcgacactggcctgccgatgagcctgtccatgaacgtctatgcgaaataccagtggcagaactatggcgcagcgaacgaaaacgagtgggacggttaccgtttcaaaattaaatactttgtgccgattaccgatctgtggggcggtcagctgagctacatcggcttcaccaacttcgactggggttccgatttaggggatgacagcggtaacgcaatcaacggtattaagacccgtactaataactctatcgcttccagccatattctggctctgaactacgatcactggcactactctgtcgtagctcgttactggcacgacggtggtcagtggaacgacgatgcagaactgaacttcggcaacggcaacttcaacgttcgctctaccggctggggtggttacctggtagtaggttacaacttctga

SEQ ID NO: 22 - protein, 294 aa, Escherichia coli
MKKTLLAAGAVLALSSSFTVNAAENDKPQYLSDWWHQSVNVVGSYHTRFGPQIRNDTYLEYEAFAKKDWFDFYGYADAPVFFGGNSDAKGIWNHGSPLFMEIEPRFSIDKLTNTDLSFGPFKEWYFANNYIYDMGRNKDGRQSTWYMGLGTDIDTGLPMSLSMNVYAKYQWQNYGAANENEWDGYRFKIKYFVPITDLWGGQLSYIGFTNFDWGSDLGDDSGNAINGIKTRTNNSIASSHILALNYDHWHYSVVARYWHDGGQWNDDAELNFGNGNFNVRSTGWGGYLVVGYNF

SEQ ID NO: 23 - DNA, 2241 bp, Escherichia coli
atgaacaagaagattcattccctggccttgttggtcaatctggggatttatggggtagcgcaggcacaagagccgaccgatactcctgtttcacatgacgatactattgtcgttaccgccgccgagcagaacttacaggcgcctggcgtttcgaccatcaccgcagatgaaatccgcaaaaacccggttgcccgcgatgtgtcgaagatcatccgtaccatgccaggcgttaacctgaccggtaactccaccagtggtcagcgtgggaataaccgacagattgatattcgcggtatgggtccggaaaacacgctgattttgattgacggcaagccggtaagcagccgtaactcggtgcgtcagggctggcgtggcgagcgcgatacccgtggtgatacttcctgggtgccacctgaaatgattgaacgtattgaagttctgcgtggtccggcagctgcgcgttatggcaacggcgcggcgggcggcgtggttaacatcattaccaaaaaaggcagcggcgagtggcacggctcctgggacgcatatttcaatgcgccagaacataaagaggaaggtgccaccaaacgcactaactttagcctgaccggtccgctgggcgacgaattcagcttccgtttgtatggcaacctcgacaaaacccaggctgacgcgtgggatatcaaccagggccatcagtccgcgcgtgccggaacgtatgccacgacgttaccagccgggcgcgaaggggtaatcaacaaagatattaatggcgtggtgcgctgggatttcgcgccattgcaatcgctggaactggaagcaggttacagccgccagggtaacctgtatgcgggcgacacccagaataccaactccgattcctatacccgctcgaaatatggcgatgaaaccaaccgtctgtatcgccagaactacgcgctgacctggaacggtggctgggataacggcgtgaccaccagcaactgggtgcagtacgaacacacccgtaactcgcgtattccggaaggtctggcgggcggtaccgaagggaaatttaacgaaaaagcgacacaggatttcgtcgatatcgatcttgatgacgtgatgctgcacagcgaagttaacctgccgattgatttcctcgttaaccagacgctgacgctgggtacggagtggaatcagcaacggatgaaggacttaagttccaacacccaggcactgaccggaacgaataccggtggcgctattgatggcgtgagtaccaccgaccgtagcccgtattcaaaagcagaaattttctcgctgtttgccgaaaacaacatggagctgactgacagcaccatcgtaacgccggggctgcgtttcgatcatcacagtattgtcggcaataactggagcccggcgctgaacatatcgcaaggtttaggcgatgacttcacgctgaaaatgggcatcgcccgtgcttataaagcgccgagcctgtaccagactaacccgaactacattctctacagtaaaggtcagggttgctatgccagcgcgggcggctgctatctgcaaggtaacgatgacctgaaagcagaaaccagcatcaacaaagagattggtctggagttcaaacgcgacgggtggctggcgggcgtcacctggttccgtaacgattatcgcaataagattgaagcgggctatgtggctgtagggcaaaacgcagtcggcaccgatctctatcagtgggataacgtgccgaaagcggtggttgaaggtctggaaggatcgttaaacgtaccggttagcgaaacggtgatgtggaccaataacatcacttatatgctgaagagtgaaaacaaaaccacgggcgaccgtttgtcgatcatcccggagtatacgttgaactcaacgctgagctggcaggcacgggaagatttgtcgatgcaaacgaccttcacctggtacggcaagcagcagccgaagaagtacaactataaaggtcagccagcggttggaccggaaaccaaagaaattagtccttacagcattgttggcctgagcgcgacctgggatgtgacgaagaatgtcagtctgaccggcggcgtggacaatctgttcgacaaacgtttgtggcgtgcgggtaatgcccagaccacgggcgatttggcaggggccaactatatcgccggtgccggggcgtatacctataacgagccgggacgtacgtggtatatgagcgtaaacacccacttctga

SEQ ID NO: 24 - protein, 746 aa, Escherichia coli
MNKKIHSLALLVNLGIYGVAQAQEPTDTPVSHDDTIVVTAAEQNLQAPGVSTITADEIRKNPVARDVSKIIRTMPGVNLTGNSTSGQRGNNRQIDIRGMGPENTLILIDGKPVSSRNSVRQGWRGERDTRGDTSWVPPEMIERIEVLRGPAAARYGNGAAGGVVNIITKKGSGEWHGSWDAYFNAPEHKEEGATKRTNFSLTGPLGDEFSFRLYGNLDKTQADAWDINQGHQSARAGTYATTLPAGREGVINKDINGVVRWDFAPLQSLELEAGYSRQGNLYAGDTQNTNSDSYTRSKYGDETNRLYRQNYALTWNGGWDNGVTTSNWVQYEHTRNSRIPEGLAGGTEGKFNEKATQDFVDIDLDDVMLHSEVNLPIDFLVNQTLTLGTEWNQQRMKDLSSNTQALTGTNTGGAIDGVSTTDRSPYSKAEIFSLFAENNMELTDSTIVTPGLRFDHHSIVGNNWSPALNISQGLGDDFTLKMGIARAYKAPSLYQTNPNYILYSKGQGCYASAGGCYLQGNDDLKAETSINKEIGLEFKRDGWLAGVTWFRNDYRNKIEAGYVAVGQNAVGTDLYQWDNVPKAVVEGLEGSLNVPVSETVMWTNNITYMLKSENKTTGDRLSIIPEYTLNSTLSWQAREDLSMQTTFTWYGKQQPKKYNYKGQPAVGPETKEISPYSIVGLSATWDVTKNVSLTGGVDNLFDKRLWRAGNAQTTGDLAGANYIAGAGAYTYNEPGRTWYMSVNTHF

SEQ ID NO: 25 - DNA, 2103 bp, Escherichia coli
atgaagattttttccgtccgacagaccgttttgcccgcactgcttgtcctttcccccgttgtttttgccgctgatgaacagactatgattgtcagtgccgcaccgcaggtggtttcagaactggataccccagcagcagtaagcgtggtggatggcgaggagatgcgcctggcaacaccgcgcattaacttgtccgaatcactgaccggcgtgcctggtttgcaggtacaaaaccggcagaactatgcgcaagatttacagctgtcgattcgcggatttggctcccgctccacttacggtattcgcggtattcgcctgtatgtggacggtattcccgccaccatgcccgacgggcaagggcaaacatccaacatcgatttaagcagtgtgcaaaatgtggaagtgctgcgtggccccttctctgccctgtatggcaacgcgtctggtggggtaatgaatgtcaccacccagaccggacaacagccaccaaccattgaagccagtagttactacggcagttttggcagctggcgctatgggctgaaagcaacgggcgcaacgggagacggcacacagcctggcgatgtcgattacaccgtctcaaccacgcgttttacgacccacggctatcgtgaccatagtggcgcacagaaaaatttagccaatgccaaactgggcgtacgcattgatgaagccagcaaattaagtctgattttcaatagtgtggatatcaaagcagatgacccaggtgggctaaccaaagcagaatggaaggctaatccacaacaagcgcctcgtgcagaacagtacgacacgcgaaaaaccatcaagcaaactcaggctgggttgcgctatgagcgtagcctgagttcgcgggatgatatgagtgtgatgatgtatgccggagagcgagaaacgacccagtaccagtcaatacccatggcaccacaacttaacccgtcacatgcgggcggcgtgattaccctgcaacgccattaccagggaatagacagccgctggacacaccgtggtgaactgggcgttccggtcacgttcactaccggcctgaactacgaaaacatgagtgaaaaccgcaagggctacaataacttccgcctgaatagcggcatgccggagtacgggcaaaaaggtgagttgcgtcgcgacgaacgcaatctgatgtggaacatcgatccctatttacagacgcagtggcagctgagcgaaaaactgtcgctggatgctggcgtgcgctacagctccgtgtggtttgattccaacgaccattacgttactccgggtaacggcgatgacagcggtgatgccagttatcataaatggctacctgccggttcgttaaaatatgcaatgaccgatgcctggaatatctatctggcagccgggcgaggttttgaaacgccgacgattaatgagctgtcttatcgtgctgatgggcaaagcggtatgaacttaggtttaaaaccatccaccaacgatacaattgagatcggcagtaaaacgcgtattggtgatgggctgcttagtctcgcattgtttcagaccgacactgatgatgaaattgttgtcgatagcagtagcggtgggcgtacgacttacaaaaatgccggaaagacccgtcgtcaaggcgctgaactggcatgggatcaacgtttcgcaggagattttcgcgtaaacgcgtcctggacctggcttgatgcgacctatcgcagcaatgtttgcaatgaacaggattgtaacggtaatcggatgccagggatcgcccgtaatatgggctttgcgtcgataggttatgtaccggaagatggttggtatgcaggcacggaagcgcgttatatgggcgatattatggcagatgatgaaaatacggcaaaagcgccgtcttatactctcgtcggcttattcaccgggtataaatacaattaccacaatttaactgtggatttatttggtcgtgtcgataatttattcgataaagaatacgttggttctgtcattgtcaatgagtcaaacgggcgatattacgaaccttcgcccggacgaaattatggtgtcggcatgaatattgcgtggagatttgagtaa

SEQ ID NO: 26 - protein, 700 aa, Escherichia coli
MKIFSVRQTVLPALLVLSPVVFAADEQTMIVSAAPQVVSELDTPAAVSVVDGEEMRLATPRINLSESLTGVPGLQVQNRQNYAQDLQLSIRGFGSRSTYGIRGIRLYVDGIPATMPDGQGQTSNIDLSSVQNVEVLRGPFSALYGNASGGVMNVTTQTGQQPPTIEASSYYGSFGSWRYGLKATGATGDGTQPGDVDYTVSTTRFTTHGYRDHSGAQKNLANAKLGVRIDEASKLSLIFNSVDIKADDPGGLTKAEWKANPQQAPRAEQYDTRKTIKQTQAGLRYERSLSSRDDMSVMMYAGERETTQYQSIPMAPQLNPSHAGGVITLQRHYQGIDSRWTHRGELGVPVTFTTGLNYENMSENRKGYNNFRLNSGMPEYGQKGELRRDERNLMWNIDPYLQTQWQLSEKLSLDAGVRYSSVWFDSNDHYVTPGNGDDSGDASYHKWLPAGSLKYAMTDAWNIYLAAGRGFETPTINELSYRADGQSGMNLGLKPSTNDTIEIGSKTRIGDGLLSLALFQTDTDDEIVVDSSSGGRTTYKNAGKTRRQGAELAWDQRFAGDFRVNASWTWLDATYRSNVCNEQDCNGNRMPGIARNMGFASIGYVPEDGWYAGTEARYMGDIMADDENTAKAPSYTLVGLFTGYKYNYHNLTVDLFGRVDNLFDKEYVGSVIVNESNGRYYEPSPGRNYGVGMNIAWRFE

SEQ ID NO: 27 - DNA, 1056 bp, Escherichia coli
atgaaaaagagcactctggcattagtggtgatgggcattgtggcatctgcatctgtacaggctgcagaaatatataataaagacggtaataaactggatgtctatggcaaagttaaagccatgcattatatgagtgataacgccagtaaagatggcgaccagagttatatccgttttggtttcaaaggcgaaacacaaattaacgatcaactgactggttatggtcgttgggaagcagagtttgccggtaataaagcagagagtgatactgcacagcaaaaaacgcgtctcgcttttgccgggttgaaatataaagatttgggttctttcgattatggtcgtaacctgggggcgttgtatgacgtggaagcctggaccgatatgttcccggaatttggtggcgattcctcggcgcagaccgacaactttatgaccaaacgcgccagcggtctggcgacgtatcggaacaccgacttcttcggcgttatcgatggcctgaacttaaccctgcaatatcaagggaaaaacgaaaaccgcgacgttaaaaagcaaaacggcgatggcttcggcacgtcattgacatatgactttggcggcagcgatttcgccattagtggggcctataccaactcagatcgcaccaacgagcagaacctgcaaagccgtggcacaggcaagcgtgcagaagcatgggcaacaggtctgaaatacgatgccaataatatttatctggcaactttctattctgaaacacgcaaaatgacgccaataactggcggctttgccaataagacacagaactttgaagcggtcgctcaataccagtttgactttggtctgcgtccatcgctgggttatgtcttatcgaaagggaaagatattgaaggtatcggtgatgaagatctggtcaattatatcgacgtcggtgctacgtattatttcaacaaaaatatgtcagcgtttgttgattataaaatcaaccaactggatagcgataacaaattgaatattaataatgatgatattgtcgcggttggcatgacgtatcagttttaa

SEQ ID NO: 28 - protein, 351 aa, Escherichia coli
MKKSTLALVVMGIVASASVQAAEIYNKDGNKLDVYGKVKAMHYMSDNASKDGDQSYIRFGFKGETQINDQLTGYGRWEAEFAGNKAESDTAQQKTRLAFAGLKYKDLGSFDYGRNLGALYDVEAWTDMFPEFGGDSSAQTDNFMTKRASGLATYRNTDFFGVIDGLNLTLQYQGKNENRDVKKQNGDGFGTSLTYDFGGSDFAISGAYTNSDRTNEQNLQSRGTGKRAEAWATGLKYDANNIYLATFYSETRKMTPITGGFANKTQNFEAVAQYQFDFGLRPSLGYVLSKGKDIEGIGDEDLVNYIDVGATYYFNKNMSAFVDYKINQLDSDNKLNINNDDIVAVGMTYQF

SEQ ID NO: 29 - DNA, 2973 bp, Escherichia coli
atgaaggagaataaccttaatcgcgtcatcggatggtctggtttactgctgacgtctttattgagtaccagcgcactcgcagacaatatcggcaccagcgcagaagagctggggctgagcgattatcgccattttgttatttatccccgtctcgataaagcgctgaaggcacagaaaaataacgacgaagcaaccgccatccgcgaatttgaatatatacaccagcaggtgccggataatattccgctgactttataccttgcggaagcctatcgccattttggtcatgatgaccgggcgcggctgttgcttgaggatcaactgaaacgtcacccaggagatgcccgacttgagcgcagtctggcggctattccggttgaagtgaaaagcgttacgacagttgaagaactgcttgcccagcaaaaagcgtgcgatgctgcgccgaccctgcgttgtcgcagtgaagtcgggcagaatgccctgcggctggcacagttacctgtcgccagagcgcaactgaacgatgcgacgtttgctgcatcgccggaaggaaaaacgctgcgaaccgatctgctgcaacgggcaatctacctgaaacaatggtcccaggcagatacgctatacaatgaagcacgccagcagaacacattaagcgcggcagaacgccgtcagtggtttgacgtgcttcttgccgggcagctggacgatcggatcctggcactgcaatcacaggggatcttcaccgatcctcagtcatatattacttacgcgaccgcgctggcttatcgtggcgaaaaagcacgcctccagcattatctcattgaaaataagccactatttaccacggacgcacaagagaaaagttggctctatctgttatctaaatacagcgctaaccccgttcaggcgttggcgaattatacggtacagtttgccgacaaccgccagtatgttgttggcgcgacgctaccggtgctgttaaaagaaggtcagtacgacgcagcgcaaaaactgctcgccaccctccccgccaatgaaatgcttgaggagcgttatgctgtcagcgtggcgacccgtaacaaggctgaagctctgcgtctggcacgattgctgtatcagcaagaaccggcaaatcttacccgcctggatcaactaacctggcaactgatgcagaacgagcagtcacgcgaagctgccgatttattgctgcaacgctatcctttccagggcgatgcgcgtgtcagccagactttaatggcgcgactggcgtctctgctggaaagtcatccttacctggcaacgccggcgaaggtggcgattttatcgaaacccttaccgctggcggagcaacgtcagtggcaaagtcagttgccgggtattgcagataattgcccggcaatagttcgcttgctgggcgatatgtcgccttcctacgatgccgccgcctggaaccgtctggcaaagtgttatcgggacacgctacccggtgtggcgttgtatgcatggcttcaggccgaacaacgacaaccgagcgcctggcaacatcgtgcggtagcctatcaggcgtatcaggttgaggactacgccaccgcactggcggcctggcagaaaatcagtcttcacgacatgagcaatgaggatctgcttgctgctgccaataccgcccaggcggcaggaaatggtgcggctcgcgatcgctggctacaacaggcagaaaaacgtggactgggaagcaatgccctctactggtggctgcatgcgcaacgttacattcctggtcagccggaactcgcactgaacgatctcacgcgctcaatcaatattgcgccttctgccaacgcttacgttgcgcgggcgacaatttatcgccaacgtcataatgtcccggccgcggtgagtgatttgcgcgccgcgctggaactggaaccgaataatagcaacacccaggcagcgcttggttacgccttgtgggatagcggtgatatcgcacagtcgcgggaaatgctcgaaccggcgcataaagggcttccggacgatccggcactgatccgacaactggcctacgtgaaccagcgtctggatgacatgcctgcgacgcagcactacgcccggctggtgattgatgacattgataatcaggcgctgataaccccactgaccccagaacaaaatcaacaacgcttcaatttccgccgtttgcatgaggaggtcggtcgccgctggacgttcagtttcgattcttccatcggcttgcgttccggcgcaatgagtaccgctaacaataatgtcggcggcgcagcgccagggaaaagctatcgtagctacggacaactggaagccgagtaccgcatcggacgcaatatgctgctggaaggcgacctgctctcagtttatagccgcgtctttgccgataccggagaaaacggggtgatgatgccggtgaaaaatccgatgtccggcaccggtctgcgctggaagccgctgcgcgatcagatctttttcatcgccgtcgaacagcagttgccgctgaacggccaaaatggcgcatccgataccatgctgcgcgccagcgcctcattctttaatggcggcaaatacagcgacgaatggcacccgaacggttcaggctggtttgcccaaaacctgtacctcgatgcggcgcaatatatccgccaggatattcaggcgtggacggcagattatcgcgtcagctggcatcagaaggtagctaacggacagactattgagccttacgctcacgttcaggacaacggctatcgtgataaaggcactcagggcgcgcagcttggcggagtcggggtccgctggaatatctggaccggcgagacgcactacgacgcctggccgcacaaagtcagtctcggcgtcgagtatcaacatacctttaaggcgattaatcaacgtaacggagagcgcaacaacgcgtttctcaccattggagtgcactggtaa

SEQ ID NO: 30 - protein, 990 aa, Escherichia coli
MKENNLNRVIGWSGLLLTSLLSTSALADNIGTSAEELGLSDYRHFVIYPRLDKALKAQKNNDEATAIREFEYIHQQVPDNIPLTLYLAEAYRHFGHDDRARLLLEDQLKRHPGDARLERSLAAIPVEVKSVTTVEELLAQQKACDAAPTLRCRSEVGQNALRLAQLPVARAQLNDATFAASPEGKTLRTDLLQRAIYLKQWSQADTLYNEARQQNTLSAAERRQWFDVLLAGQLDDRILALQSQGIFTDPQSYITYATALAYRGEKARLQHYLIENKPLFTTDAQEKSWLYLLSKYSANPVQALANYTVQFADNRQYVVGATLPVLLKEGQYDAAQKLLATLPANEMLEERYAVSVATRNKAEALRLARLLYQQEPANLTRLDQLTWQLMQNEQSREAADLLLQRYPFQGDARVSQTLMARLASLLESHPYLATPAKVAILSKPLPLAEQRQWQSQLPGIADNCPAIVRLLGDMSPSYDAAAWNRLAKCYRDTLPGVALYAWLQAEQRQPSAWQHRAVAYQAYQVEDYATALAAWQKISLHDMSNEDLLAAANTAQAAGNGAARDRWLQQAEKRGLGSNALYWWLHAQRYIPGQPELALNDLTRSINIAPSANAYVARATIYRQRHNVPAAVSDLRAALELEPNNSNTQAALGYALWDSGDIAQSREMLEPAHKGLPDDPALIRQLAYVNQRLDDMPATQHYARLVIDDIDNQALITPLTPEQNQQRFNFRRLHEEVGRRWTFSFDSSIGLRSGAMSTANNNVGGAAPGKSYRSYGQLEAEYRIGRNMLLEGDLLSVYSRVFADTGENGVMMPVKNPMSGTGLRWKPLRDQIFFIAVEQQLPLNGQNGASDTMLRASASFFNGGKYSDEWHPNGSGWFAQNLYLDAAQYIRQDIQAWTADYRVSWHQKVANGQTIEPYAHVQDNGYRDKGTQGAQLGGVGVRWNIWTGETHYDAWPHKVSLGVEYQHTFKAINQRNGERNNAFLTIGVHW

SEQ ID NO: 31 - protein, 11 aa, Artificial sequence
  in-frame 11 amino acids extension in the tolC protein sequence (SEQ ID NO 12)
VGLSFSLPIYQ

SEQ ID NO: 32 - DNA, 1515 bp, Escherichia coli
atgaagaaattgctccccattcttatcggcctgagcctttctgggttcagttcgttgagccaggccgagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccttgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcaggttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactga

SEQ ID NO: 33 - protein, 504 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQTLILNTATAYFNVLNAIDVLSYTQAQKEAIYRQLDQTTQRFNVGLVAITDVQNARAQYDTVLANEVTARNNLDNAVEQLRQITGNYYPELAALNVENFKTDKPQPVNALLKEAEKRNLSLLQARLSQDLAREQIRQAQDGHLPTLDLTASTGISDTSYSGSKTRGAAGTQYDDSNMGQNKVGLSFSLPIYQVGLSFSLPIYQGGMVNSQVKQAQYNFVGASEQLESAHRSVVQTVRSSFNNINASISSINAYKQAVVSAQSSLDAMEAGYSVGTRTIVDVLDATTTLYNAKQELANARYNYLINQLNIKSALGTLNEQDLLALNNALSKPVSTNPENVAPQTPEQNAIADGYAPDSPAPVVQQTSARTTTSNGHNPFRN

SEQ ID NO: 34 - DNA, 2259 bp, Escherichia coli
atgaagaaattgctccccattcttatcggcctgagcctttctgggttcagttcgttgagccaggccgagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccttgcagttaactcaatccatttttgatatgtcgaaatggcgtgcgttaacgctgcaggaaaaagcagcagggattcaggacgtcacgtatcagaccgatcagcaggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactga

SEQ ID NO: 35 - protein, 131 aa, Escherichia coli
MKKLLPILIGLSLSGFSSLSQAENLMQVYQQARLSNPELRKSAADRDAAFEKINEARSPLLPQLGLGADYTYSNGYRDANGINSNATSASLQLTQSIFDMSKWRALTLQEKAAGIQDVTYQTDQQVMLPTY

SEQ ID NO: 36 - DNA, 900 bp, Escherichia coli
atggcctttaaagttgttcagatttgtggtggtctgggcaatcagatgtttcagtatgcatttgcaaaaagcctgcagaaacatagcaatacaccggttctgctggatattaccagctttgattggagcaatcgtaaaatgcagctggaactgtttccgattgatctgccatatgcaagcgaaaaagaaattgcaattgccaaaatgcagcatctgccgaaactggttcgtaatgttctgaaatgcatgggttttgatcgtgtgagccaagaaatcgtgtttgaatatgaaccgaaactgctgaaaaccagccgtctgacctatttttatggctattttcaggatccgcgttattttgatgcaattagtccgctgatcaaacagacctttaccctgcctccgcctccggaaaatggtaataacaaaaaaaaagaagaagagtatcatcgtaaactggcactgattctggcagcaaaaaatagcgtgtttgtgcatattcgtcgcggtgattatgttggtattggttgtcagctgggcatcgattatcagaaaaaagcactggaatacatggcaaaacgtgttccgaatatggaactgtttgtgttttgcgaggacctggaatttacccagaatctggatctgggctatccgtttatggatatgaccacccgtgataaagaggaagaggcatattgggatatgctgctgatgcagagctgtaaacatggtattattgccaacagcacctatagttggtgggcagcatatctgattaataacccggaaaaaatcattattggtccgaaacattggctgtttggccatgaaaacatcctgtgtaaagaatgggtgaaaatcgaaagccactttgaagtgaaaagccagaaatataatgcctaa

SEQ ID NO: 37 - protein, 299 aa, Escherichia coli
MAFKVVQICGGLGNQMFQYAFAKSLQKHSNTPVLLDITSFDWSNRKMQLELFPIDLPYASEKEIAIAKMQHLPKLVRNVLKCMGFDRVSQEIVFEYEPKLLKTSRLTYFYGYFQDPRYFDAISPLIKQTFTLPPPPENGNNKKKEEEYHRKLALILAAKNSVFVHIRRGDYVGIGCQLGIDYQKKALEYMAKRVPNMELFVFCEDLEFTQNLDLGYPFMDMTTRDKEEEAYWDMLLMQSCKHGIIANSTYSWWAAYLINNPEKIIIGPKHWLFGHENILCKEWVKIESHFEVKSQKYNA

SEQ ID NO: 38 - DNA, 990 bp, Basilea psittacipulmonis
atgtcagtgctgaaaaaactggtaagaacgctgaaaaagaaaaaagatattcccagcgaaaatcaggaagatattaagccgcaggaatttggacatatcaaacattatcatttctggcctctgtcgaacgaaacgttttttaaccaatttgcgcaggaaaaaaaccttgatctgagccagacggccctaattagctgctttggggaactatcagcgattcctaaaatccctgaacggtataaagtgttttttacgggggaaaatatctatcatccggatcgcatctcatatgcggatccggagctctatcgcatggtggatctgtatttaggctttgaataccggacggaaccgaagtatctacgctttccgctgtgggtttggtatttatgcggcctgacgaaaaaaccgcatttttcacatgaatcaattgcggaatttattcggaaaatgaaccaacctgagtttcgcctgcagtcatcaaggaatcgcttttgcagccatattagcagccatgatacgaacggcattcggaaacggatgattgatttaattttaccgattgcgtcagtggattgcgccggcaagtttatgaacaacacggatgagttaaaagcgaagtttaatgatgacaagatcgactatctaaaacagtatcggtttaatctctgtcctgaaaactcagaatcagtcgggtatatcacggaaaagatttttgaatcaattatggccgggtgcattccaatttattggggaggagtcaagcagctttttgtcgaaccggatattttaaatccggaagcatttatttactacgaaaaagggaaagaagagcaattagcgaaacaggttgaagaactttggatatcacctaaacggtatgaagagtttgcagcaattgccccgtttaaagaggacgcagccgaagtgatttatacgtggattgaagaactggaaaaacggctacgggcatttgaaccaaaagcctaa

SEQ ID NO: 39 - protein, 329 aa, Basilea psittacipulmonis
MSVLKKLVRTLKKKKDIPSENQEDIKPQEFGHIKHYHFWPLSNETFFNQFAQEKNLDLSQTALISCFGELSAIPKIPERYKVFFTGENIYHPDRISYSDPELYRMVDLYLGFEYRTEPKYLRFPLWVWYLCGLTKKPHFSHESIAEFIRKMNQPEFRLQSSRNRFCSHISSHDTNGIRKRMIDLILPIASVDCAGKFMNNTDELKAKFNDDKIDYLKQYRFNLCPENSESVGYITEKIFESIMAGCIPIYWGGVKQLFVEPDILNPEAFIYYEKGKEEQLAKQVEELWISPKRYEEFAAIAPFKEDAAEVIYTWIEELEKRLRAFEPKA

SEQ ID NO: 40 - DNA, 978 bp, Azospirillum oryzae
atgattgatcagcgtaccagcgactttctaagcgagttcctggcgtcaagccaccgtgatccggcgcgtttagatagcttcctgcttcacgggccaggccgcggcgctcgcgcggcgaaaccgcgtctgaaaattgcgttttttgacttttggccggaatttgacccggctgcgaacttttttgtggacattctgagcgcgcgctttgatgtaagcgttgtggataacgatagcgacttagcgattgtgagcgtgtttggcacccgccatcgcgaagcgcgtaccgcgcgtagcatgttttttaccggcgaaaatgtgcgtccgccgttagatggcgtggatatgagcgtgagctttgatcgcattgatgacccacgccactatcgcctgccgttatatgtgatgcacgcgtgggatcataggcgtgaaggggcgacgccgcacttttgccagagcgtgctgccgccggtgccgccgacgcgtgaagaagcggcgaaacgtaagttttgcgcgtttttatataaaaatccaaactgcgcgaggcgcaacgactttttccagatgttatgcgcgaggcgccacgtggaaagcgtgggctggctgctgaataataccggcagcgtggtgaaaatgggctggctgccgaaaattcgtgtgttcagccgctaccgctttgcgtttgcgtttgaaaatgcgagccacccaggctacctgacggagaaaattttagatgcgtttcaagcgggcgcggtgccgttatattggggcgacccaggagtgctgcgcgacgttgcggccggcagctttattgatgtgagccgctacgcgagcgatgaagaagcgtgcgacgcgattctggccgcggatgatgattacgatacctaccgccgctaccgcagcacgccgccgtttctgggcgcggaagatttttactttgatgcgtttcggctggccgagtggattgagagccgtctataa

SEQ ID NO: 41 - protein, 325 aa, Azospirillum oryzae
MIDQRTSDFLSEFLASSHRDPARLDSFLLHGPGRGARAAKPRLKIAFFDFWPEFDPAANFFVDILSARFDVSVVDNDSDLAIVSVFGTRHREARTARSMFFTGENVRPPLDGVDMSVSFDRIDDPRHYRLPLYVMHAWDHRREGATPHFCQSVLPPVPPTREEAAKRKFCAFLYKNPNCARRNDFFQMLCARRHVESVGWLLNNTGSVVKMGWLPKIRVFSRYRFAFAFENASHPGYLTEKILDAFQAGAVPLYWGDPGVLRDVAAGSFIDVSRYASDEEACDAILAADDDYDTYRRYRSTPPFLGAEDFYFDAFRLAEWIESRL

SEQ ID NO: 42 - DNA, 1665 bp, Escherichia coli
atggcgcgttccaaaactgctcagccaaaacactcactgcgtaaaatcgcagttgtagtagccacagcggttagcggcatgtctgtttatgcacaggcagcggttgaaccgaaagaagacactatcaccgttaccgctgcacctgcgccgcaagaaagcgcatgggggcctgctgcaactattgcggcgcgacagtctgctaccggcactaaaaccgatacgccgattcaaaaagtgccacagtctatttctgttgtgaccgccgaagagatggcgctgcatcagccgaagtcggtaaaagaagcgcttagctacacgccgggtgtctctgttggtacgcgtggcgcatccaacacctatgaccacctgatcattcgcggctttgcggcagaaggccaaagccagaataactatctgaatggcctgaagttgcagggcaacttctataacgatgcggtcattgacccgtatatgctggaacgcgctgaaattatgcgtggcccggtttccgtgctttacggtaaaagcagtcctggcggcctgttgaatatggtcagcaagcgtccgaccaccgaaccgctgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtattag

SEQ ID NO: 43 - protein, 554 aa, Escherichia coli
MARSKTAQPKHSLRKIAVVVATAVSGMSVYAQAAVEPKEDTITVTAAPAPQESAWGPAATIAARQSATGTKTDTPIQKVPQSISVVTAEEMALHQPKSVKEALSYTPGVSVGTRGASNTYDHLIIRGFAAEGQSQNNYLNGLKLQGNFYNDAVIDPYMLERAEIMRGPVSVLYGKSSPGGLLNMVSKRPTTEPLKEVQFKAGTDSLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQY

SEQ ID NO: 44 - DNA, 2227 bp, Escherichia coli
atggcgcgttccaaaactgctcagccaaaacactcactgcgtaaaatcgcagttgtagtagccacagcggttagcggcatgtctgtttatgcacaggcagcggttgaaccgaaagaagacactatcaccgttaccgctgcacctgcgccgcaagaaagcgcatgggggcctgctgcaactattgcggcgcgacagtctgctaccggcactaaaaccgatacgccgattcaaaaagtgccacagtctatttctgttgtgaccgccgaagagatggcgctgcatcagccgaagtcggtaaaagaagcgcttagctacacgccgggtgtctctgttggtacgcgtggcgcatccaacacctatgaccacctgatcattcgcggctttgcggcagaaggccaaagccagaataactatctgaatggcctgaagttgcagggcaacttctataacgatgcggtcattgacccgtatatgctggaacgcgctgaaattatgcgtggcccggtttccgtgctttacggtaaaagcagtcctggcggcctgttgaatatggtcagcaagcgtccgaccaccgaaccgctgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaaggaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 45 - protein, 568 aa, Escherichia coli
MARSKTAQPKHSLRKIAVVVATAVSGMSVYAQAAVEPKEDTITVTAAPAPQESAWGPAATIAARQSATGTKTDTPIQKVPQSISVVTAEEMALHQPKSVKEALSYTPGVSVGTRGASNTYDHLIIRGFAAEGQSQNNYLNGLKLQGNFYNDAVIDPYMLERAEIMRGPVSVLYGKSSPGGLLNMVSKRPTTEPLKEVQFKAGTDSLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKEICTGRSSDCSYWCRV

SEQ ID NO: 46 - DNA, 3580 bp, Escherichia coli
atggcgcgttccaaaactgctcagccaaaacactcactgcgtaaaatcgcagttgtagtagccacagcggttagcggcatgtctgtttatgcacaggcagcggttgaaccgaaagaagacactatcaccgttaccgctgcacctgcgccgcaagaaagcgcatgggggcctgctgcaactattgcggcgcgacagtctgctaccggcactaaaaccgatacgccgattcaaaaagtgccacagtctatttctgttgtgaccgccgaagagatggcgctgcatcagccgaagtcggtaaaagaagcgcttagtagactggccccctgaatctccagacaaccaatatcacttaaataagtgatagtcttaatactagtttttagactagtcattggagaacagatgattgatgtcttagggccggagaaacgcagacggcgtaccacacaggaaaagatcgcaattgttcagcagagctttgaaccggggatgacggtctccctcgttgcccggcaacatggtgtagcagccagccagttatttctctggcgtaagcaataccaggaaggaagtcttactgctgtcgccgccggagaacaggttgttcctgcctctgaacttgctgccgccatgaagcagattaaagaactccagcgcctgctcggcaagaaaacgatggaaaatgaactcctcaaagaagccgttgaatatggacgggcaaaaaagtggatagcgcacgcgcccttattgcccggggatggggagtaagcttagtcagccgttgtctccgggtgtcgcgtgcgcagttgcacgtcattctcagacgaaccgatgactggatggatggccgccgcagtcgtcacactgatgatacggatgtgcttctccgtatacaccatgttatcggagagctgccaacgtatggttatcgtcgggtatgggcgctgcttcgcagacaggcagaacttgatggtatgcctgcgatcaatgccaaacgtgtttaccggatcatgcgccagaatgcgctgttgcttgagcgaaaacctgctgtaccgccatcgaaacgggcacatacaggcagagtggccgtgaaagaaagcaatcagcgatggtgctctgacgggttcgagttctgctgtgataacggagagagactgcgtgtcacgttcgcgctggactgctgtgatcgtgaggcactgcactgggcggtcactaccggcggcttcaacagtgaaacagtacaggacgtcatgctgggagcggtggaacgccgcttcggcaacgatcttccgtcgtctccagtggagtggctgacggataatggttcatgctaccgggctaatgaaacacgccagttcgcccggatgttgggacttgaaccgaagaacacggcggtgcggagtccggagagtaacggaatagcagagagcttcgtgaaaacgataaagcgtgactacatcagtatcatgcccaaaccagacgggttaacggcagcaaagaaccttgcagaggcgttcgagcattataacgaatggcatccgcatagtgcgctgggttatcgctcgccacgggaatatctgcggcagcgggcttgtaatgggttaagtgataacagatgtctggaaatataggggcaaatccacttagctacacgccgggtgtctctgttggtacgcgtggcgcatccaacacctatgaccacctgatcattcgcggctttgcggcagaaggccaaagccagaataactatctgaatggcctgaagttgcagggcaacttctataacgatgcggtcattgacccgtatatgctggaacgcgctgaaattatgcgtggcccggtttccgtgctttacggtaaaagcagtcctggcggcctgttgaatatggtcagcaagcgtccgaccaccgaaccgctgaaagaagttcagtttaaagccggtactgacagcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtatgaagtcggcgtgaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 47 - protein, 124 aa, Escherichia coli
MARSKTAQPKHSLRKIAVVVATAVSGMSVYAQAAVEPKEDTITVTAAPAPQESAWGPAATIAARQSATGTKTDTPIQKVPQSISVVTAEEMALHQPKSVKEALSLATRRVSLLVRVAHPTPMTT

SEQ ID NO: 48 - DNA, 2169 bp, Escherichia coli
atggcgcgttccaaaactgctcagccaaaacactcactgcgtaaaatcgcagttgtagtagccacagcggttagcggcatgtctgtttatgcacaggcagcggttgaaccgaaagaagacactatcaccgttaccgctgcacctgcgccgcaagaaagcgcatgggggcctgctgcaactattgcggcgcgacagtctgctaccggcactaaaaccgatacgccgattcaaaaagtgccacagtctatttctgttgtgaccgccgaagagatggcgctgcatcagccgaagtcggtaaaagaagcgcttagctacacgccgggtgtctctgttggtacgcgtggcgcatccaacacctatgaccacctgatcattcgcggctttgcggcagaaggccaaagccagaataactatctgaatggcctgaagttgcagggcaacttctataacgatgcggtcattgacccgtatatgctggaacgcgctgaaattatgcgtggcccggtttccgtgctttacggtaaaagcagtcctggcggcctgttccagactggttttgactttagcgattcgttggatgatgacggtgtttactcttatcgcctgaccggtcttgcgcgttctgccaatgcccagcagaaagggtcagaagagcagcgttatgctattgcaccggcgttcacctggcgtccggatgataaaaccaattttaccttcctttcttacttccagaacgagccggaaaccggttattacggctggttgccgaaagagggaaccgttgagccgctgccgaacggtaagcgtctgccgacagactttaatgaaggggcgaagaacaacacctattctcgtaatgagaagatggtcggctacagcttcgatcacgaatttaacgacacctttactgtgcgtcagaacctgcgctttgctgaaaacaaaacctcgcaaaacagcgtttatggttacggcgtctgctccgatccggcgaatgcttacagcaaacagtgtgcggcattagcgccagcggataaaggccattatctggcacgtaaatacgtcgttgatgatgagaagctgcaaaacttctccgttgatacccagttgcagagcaagtttgccactggcgatatcgaccacaccctgctgaccggtgtcgactttatgcgtatgcgtaatgacatcaacgcctggtttggttacgacgactctgtgccactgctcaatctgtacaatccggtgaataccgatttcgacttcaatgccaaagatccggcaaactccggcccttaccgcattctgaataaacagaaacaaacgggcgtttatgttcaggatcaggcgcagtgggataaagtgctggtcaccctaggcggtcgttatgactgggcagatcaagaatctcttaaccgcgttgccgggacgaccgataaacgtgatgacaaacagtttacctggcgtggtggtgttaactacctgtttgataatggtgtaacaccttacttcagctatagcgaatcgtttgaaccttcttcgcaagttgggaaggatggtaatattttcgcaccgtctaaaggtaagcagtatgaagtcggcgtgaaatatgtaccggaagatcgtccgattgtagttactggtgccgtgtataatctcactaaaaccaacaacctgatggcggaccctgagggttccttcttctcggttgaaggtggcgagatccgcgcacgtggcgtagaaatcgaagcgaaagcggcgctgtcggcgagtgttaacgtagtcggttcttatacttacaccgatgcggaatacaccaccgatactacctataaaggcaatacgcctgcacaggtgccaaaacacatggcttcgttgtgggctgactacaccttctttgacggtccgctttcaggtctgacgctgggcaccggtggtcgttatactggctccagttatggtgatccggctaactcctttaaagtgggaagttatacggtcgtggatgcgttagtacgttatgatctggcgcgagtcggcatggctggctccaacgtggcgctgcatgttaacaacctgttcgatcgtgaatacgtcgccagctgctttaacacttatggctgcttctggggcgcagaacgtcaggtcgttgcaaccgcaaccttccgtttctaa

SEQ ID NO: 49 - protein, 722 aa, Escherichia coli
MARSKTAQPKHSLRKIAVVVATAVSGMSVYAQAAVEPKEDTITVTAAPAPQESAWGPAATIAARQSATGTKTDTPIQKVPQSISVVTAEEMALHQPKSVKEALSYTPGVSVGTRGASNTYDHLIIRGFAAEGQSQNNYLNGLKLQGNFYNDAVIDPYMLERAEIMRGPVSVLYGKSSPGGLFQTGFDFSDSLDDDGVYSYRLTGLARSANAQQKGSEEQRYAIAPAFTWRPDDKTNFTFLSYFQNEPETGYYGWLPKEGTVEPLPNGKRLPTDFNEGAKNNTYSRNEKMVGYSFDHEFNDTFTVRQNLRFAENKTSQNSVYGYGVCSDPANAYSKQCAALAPADKGHYLARKYVVDDEKLQNFSVDTQLQSKFATGDIDHTLLTGVDFMRMRNDINAWFGYDDSVPLLNLYNPVNTDFDFNAKDPANSGPYRILNKQKQTGVYVQDQAQWDKVLVTLGGRYDWADQESLNRVAGTTDKRDDKQFTWRGGVNYLFDNGVTPYFSYSESFEPSSQVGKDGNIFAPSKGKQYEVGVKYVPEDRPIVVTGAVYNLTKTNNLMADPEGSFFSVEGGEIRARGVEIEAKAALSASVNVVGSYTYTDAEYTTDTTYKGNTPAQVPKHMASLWADYTFFDGPLSGLTLGTGGRYTGSSYGDPANSFKVGSYTVVDALVRYDLARVGMAGSNVALHVNNLFDREYVASCFNTYGCFWGAERQVVATATFRF

SEQ ID NO: 50 - DNA, 1047 bp, Neisseria meningitidis
atgccgagcgaagcatttcgtcgtcatcgtgcatatcgtgaaaacaaactgcagccgctggttagcgttctgatttgtgcatataatgtggaaaaatactttgcccagagcctggcagcagttgttaatcagacctggtgtaatctggatattctgattgttgatgatggtagcaccgatggcaccctggcaattgcacagcgttttcaagaacaggatggtcgtatcaaaattctggcacaggcacagaatagcggtctgattccgagcctgaatattggtctggatgaactggcaaaaagcggtatgggtgaatatattgcacgtaccgatgcagatgatattgcagcaccggattggattgaaaaaattgtgggtgagatggaaaaagatcgcagcattattgcaatgggtgcatggctggaagttctgagcgaagaaaaagatggtaatcgtctggcacgtcatcatgaacatggtaaaatttggaaaaaaccgacccgtcatgaagatatcgcagatttttttccgtttggcaacccgattcataacaacaccatgattatgcgtcgtagcgttattgatggtggtctgcgttataataccgaacgtgattgggcagaagattatcagttttggtatgatgttagcaaactgggtcgtctggcctattatccggaagcactggttaaatatcgcctgcatgcaaatcaggttagcagcaaatatagcattcgccagcatgaaattgcccagggtattcagaaaaccgcacgtaatgattttctgcagagcatgggctttaaaacccgttttgatagcctggaatatcgccagattaaagcagttgcctatgaactgctggaaaaacatctgccggaagaagattttgaacgcgcacgtcgttttctgtatcagtgttttaaacgcaccgataccctgcctgccggtgtttggctggattttgcagcaaatggtcgcatgcgtcgtctgtttaccctgcgtcagtattttggtattctgcatcgtctgctgaaaaaccgctaa

SEQ ID NO: 51 - DNA, 798 bp, Escherichia oli
atgattattgatgaagcggaaagcgcggaaagcacccatccagtggtgagcgtgattctgccggtgaataaaaaaaacccgtttctggatgaagcgattaatagcattctgagccagacctttagcagctttgaaattattattgtggcgaactgctgcaccgatgatttttataacgaactgaaacataaagtgaacgataaaattaaactgattcgcaccaatattgcgtatctgccgtatagcctgaataaagcgattgatctgagcaacggcgaatttattgcgcgcatggatagcgatgatattagccatccggatcgctttaccaaacaagtggattttctgaaaaataacccgtatgttgatgttgtgggcaccaacgcgatttttattgatgataaaggccgcgaaattaataaaaccaaactgccggaagaaaacctggatattgtgaaaaacctgccgtataaatgctgcattgtgcatccgagcgtgatgtttcgcaaaaaagtgattgcgagcattggcggctatatgtttagcaactatagcgaagattatgaactgtggaaccgcctgagcctggcgaaaattaaatttcagaacctgccggaatatctgttttattatcgcctgcatgaaggccagagcaccgcgaaaaaaaacctgtatatggtgatggtgaacgatctggtgattaaaatgaaatgcttttttctgaccggcaatattaactatctgtttggcggcattcgcaccattgcgagctttatttattgcaaatatattaaataa

SEQ ID NO: 52 - DNA, 828 bp, Neisseria meningitidis
atgcagaaccatgttattagccttgctagcgcggcagaacgccgagcccatatcgcggacacctttggccgacatggcattccgttccaattctttgatgcactgatgccgtcagaacgactggaacaagcaatggccgaattagtgccaggcttatcggcccatccatacttaagcggagttgaaaaagcgtgcttcatgagccatgcggtattatggaagcaagcattagatgaagggctgccgtacattaccgtattcgaggatgatgttcttttaggcgaaggggccgaaaaatttcttgcggaagatgcttggctgcaggaacgcttcgatccggacaccgcgttcattgtgcgcttagaaacgatgttcatgcatgtgctgacctcgccaagcggcgttgccgactattgcggacgcgcgttcccgctgttagaaagcgaacattggggaacggccggctacattatcagcagaaaagccatgcggttctttctggatcgattcgcggcgctgccgccagaaggactgcatccagtggacctgatgatgtttagcgacttctttgatcgagaaggaatgccggtttgccaattaaatccagcgttatgcgcgcaggagctgcactacgcgaagttccatgatcagaacagcgcattaggcagcctgattgaacatgatcgcttactgaaccgcaaacaacagcgacgcgacagcccagcgaacacatttaaacatcgcctgattcgcgcgttaaccaaaattagccgagaacgagaaaaacgccggcagcgacgcgaacaatttatcgttccttttcagtaa

SEQ ID NO: 53 - DNA, 906 bp, Zymomonas mobilis
atgaaaaacgataaaaaaatttatggatgcattgaaggcggcggaacgaaatttatgctcgcccttattgattctgaccggaagatgctggctgtggaacgtgttccgaccacaacccctgaagaaacgcttggtaaaagtgttgaattcttcaaaaaagcgcttcctcaatacgctgattcttttgcgtcttttggtatcgcgtcatttggtccgctttgcctcgatcgcaaaagcccgaaatggggctatatcaccaataccccgaaacctttctggccgaatacggacgttgtaacgccctttaaagaagcttttggttgcccggttgaaatcgacaccgatgtgaatggcgcagctttggctgaaaacttctggggcgcatcaaaaggcacccatacatctgtttatgtcacggttggcaccggcttcggcggtggcgtactgattgatggtaaaccgattcatggtctggctcaccctgaaatgggtcatggtattccgatccgtcatcctgatgatcgtgatttcgaaggttgctgcccttatcatggcggttgctatgaaggcctcgccagcggcacggctatccgtaaacgttggggcaaggctctgaacgaaatggaaccggccgaatttgaaaaagccagagaaatcattgctttctatttggctcacttcaacgtcacgcttcaggcctttatttcaccggaacgtatcgttttcggcggcggcgttatgcatgttgacggtatgctggcttctgttcgtcgtcagacggctgaaattgccaatagttattttgaaggtgctgacttcgaaaaaattatcgtattaccagggttaggtgatcaggccggtatgatgggtgccttcgccttggcattggctgcagaaaataaataa

SEQ ID NO: 54 - DNA, 1515 bp, Bifidobacterium adolescentis
atgaaaaacaaggtgcagctcatcacttacgccgaccgccttggcgacggcaccatcaagtcgatgaccgacattctgcgcacccgcttcgacggcgtgtacgacggcgttcacatcctgccgttcttcaccccgttcgacggcgccgacgcaggcttcgacccgatcgaccacaccaaggtcgacgaacgtctgggcagctgggacgacgtcgccgaactctccaagacccacaacatcatggtcgacgccatcgtcaaccacatgagttgggaatccaagcagttccaggacgtgctggccaagggcgaggagtccgaatactatccgatgttcctcaccatgagctccgtgttcccgaacggcgccaccgaagaggacctggccggcatctaccgtccgcgtccgggcctgccgttcacccactacaagttcgccggcaagacccgcctcgtgtgggtcagcttcaccccgcagcaggtggacatcgacaccgattccgacaagggttgggaatacctcatgtcgattttcgaccagatggccgcctctcacgtcagctacatccgcctcgacgccgtcggctatggcgccaaggaagccggcaccagctgcttcatgaccccgaagaccttcaagctgatctcccgtctgcgtgaggaaggcgtcaagcgcggtctggaaatcctcatcgaagtgcactcctactacaagaagcaggtcgaaatcgcatccaaggtggaccgcgtctacgacttcgccctgcctccgctgctgctgcacgcgctgagcaccggccacgtcgagcccgtcgcccactggaccgacatacgcccgaacaacgccgtcaccgtgctcgatacgcacgacggcatcggcgtgatcgacatcggctccgaccagctcgaccgctcgctcaagggcctcgtgccggatgaggacgtggacaacctcgtcaacaccatccacgccaacacccacggcgaatcccaggcagccactggcgccgccgcatccaatctcgacctctaccaggtcaacagcacctactattcggcgctcgggtgcaacgaccagcactacatcgccgcccgcgcggtgcagttcttcctgccgggcgtgccgcaagtctactacgtcggcgcgctcgccggcaagaacgacatggagctgctgcgtaagacgaataacggccgcgacatcaatcgccattactactccaccgcggaaatcgacgagaacctcaagcgtccggtcgtcaaggccctgaacgcgctcgccaagttccgcaacgagctcgacgcgttcgacggcacgttctcgtacaccaccgatgacgacacgtccatcagcttcacctggcgcggcgaaaccagccaggccacgctgacgttcgagccgaagcgcggcctcggtgtggacaacactacgccggtcgccatgttggaatgggaggattccgcgggcgaccaccgttcggatgatctgatcgccaatccgcctgtcgtcgcctga

SEQ ID NO: 55 - DNA, 807 bp, Pasteurella multocida
atggataagttcgcggaacatgaaataccaaaagcagttattgttgcggggaacggtgagagcctgagccagattgattataggttactgccaaaaaactatgatgtgtttcgctgcaaccagttttattttgaagaacggtattttctggggaataagataaaagcggttttcttcactccgggggtctttttggagcagtattatacgttgtatcatcttaagcggaataacgagtattttgttgataacgtgattctttcgtcgtttaaccatccaacggtcgatctggaaaagagccaaaaaatacaggcgttgtttattgatgtgatcaatgggtatgaaaagtatctgtcgaaacttacggcatttgatgtttatttacggtataaagaactgtatgagaaccagcggattacgtcgggtgtctatatgtgcgcggttgcaattgccatggggtatacggatatttacctgacgggcatcgatttttatcaggcctcggaagaaaattacgcgttcgataacaaaaagccaaatattattaggctgttaccagattttagaaaagaaaaaacgcttttttcgtatcatagcaaagatattgatttagaagcgctgtcgtttctgcagcaacattatcatgttaacttttattcgatttcgccgatgtcgccattatcgaaacattttccaattccgacggtcgaggatgattgcgaaacgacgtttgttgccccgctaaaagaaaactacattaacgatatattattaccaccacattttgtctatgaaaaactggggtag

SEQ ID NO: 56 - DNA, 1179 bp, Photobacterium damselae
atgaccgttgttgcaccgaccctggaagtttatattgatcatgcaagcctgccgagcctgcagcagctgattcatattattcaggccaaagatgaatatccgagcaatcagcgttttgttagctggaaacgtgttaccgttgatgcagataatgccaacaaactgaacattcatacctatccgctgaaaggcaataataccagtccggaaatggttgcagcaattgatgaatatgcacagagcaaaaatcgcctgaacatcgaattctataccaataccgcacacgtgtttaataacctgcctccgattattcagccgctgtataataacgagaaagtgaaaattagccacatcagcctgtatgatgatggtagcagcgaatatgttagcctgtatcagtggaaagataccccgaacaaaattgaaacactggaaggtgaagttagcctgctggcaaattatctggcaggcacctcaccggatgcaccgaaaggtatgggtaatcgttataattggcacaaactgtatgacaccgactattactttctgcgcgaagattatctggatgttgaagcaaatctgcatgatctgcgtgattacctgggtagcagtgcaaaacaaatgccgtgggatgaatttgcaaaactgagcgatagccagcagaccctgtttctggatattgtgggttttgataaagagcaattgcaacaacaatattcacaatccccactaccaaactttatttttaccggcacaacaacttgggctgggggggaaacgaaagagtattatgctcagcaacaagtaaatgtgattaataatgcgatcaatgaaactagcccttattatttaggtaaagactacgatctatttttcaaggggcatcctgctggtggcgttattaacgacatcattcttggaagcttccctgatatgatcaatattccagccaagatttcatttgaggtcttgatgatgacggatatgttgcctgatacagtagctggtattgcgagctctctgtacttcacaattcctgccgataaagttaattttattgtatttacttcatctgacactattactgatcgtgaagaggctcttaaatcaccattagtacaagtgatgctaacgttgggtattgttaaagaaaaagatgttctgttctgggcttaataa

SEQ ID NO: 57 - DNA, 1830 bp, Escherichia coli
atgtgtggaattgttggcgcgatcgcgcaacgtgatgtagcagaaatccttcttgaaggtttacgtcgtctggaataccgcggatatgactctgccggtctggccgttgttgatacagaaggtcatatgacccgcctgcgtcgcctcggtaaagtccagatgctggcacaggcagcggaagaacatcctctgcatggcggcactggtattgctcacactcgctgggcgacccacggtgaaccttcagaagtgaatgcgcatccgcatgtttctgaacacattgtggtggtgcataacggcatcatcgaaaaccatgaaccgctgcgtgaagagctaaaagcgcgtggctataccttcgtttctgaaaccgacaccgaagtgattgcccatctggtgaactgggagctgaaacaaggcgggactctgcgtgaggccgttctgcgtgctatcccgcagctgcgtggtgcgtacggtacagtgatcatggactcccgtcacccggataccctgctggcggcacgttctggtagtccgctggtgattggcctggggatgggcgaaaactttatcgcttctgaccagctggcgctgttgccggtgacccgtcgctttatcttccttgaagagggcgatattgcggaaatcactcgccgttcggtaaacatcttcgataaaactggcgcggaagtaaaacgtcaggatatcgaatccaatctgcaatatgacgcgggcgataaaggcatttactgtcactacatgcagaaagagatctacgaacagccgaacgcgatcaaaaacacccttaccggacgcatcagccacggtcaggttgatttaagcgagctgggaccgaacgccgacgaactgctgtcgaaggttgagcatattcagatcctcgcctgtggtacttcttataactccggtatggtttcccgctactggtttgaatcgctagcaggtattccgtgcgacgtcgaaatcgcctctgaattccgctatcgcaaatctgccgtgcgtcgtaacagcctgatgatcaccttgtcacagtctggcgaaaccgcggataccctggctggcctgcgtctgtcgaaagagctgggttaccttggttcactggcaatctgtaacgttccgggttcttctctggtgcgcgaatccgatctggcgctaatgaccaacgcgggtacagaaatcggcgtggcatccactaaagcattcaccactcagttaactgtgctgttgatgctggtggcgaagctgtctcgcctgaaaggtctggatgcctccattgaacatgacatcgtgcatggtctgcaggcgctgccgagccgtattgagcagatgctgtctcaggacaaacgcattgaagcgctggcagaagatttctctgacaaacatcacgcgctgttcctgagccgtggcgatcagtacccaatcgcgctggaaggcgcattgaagttgaaagagatctcttacattcacgctgaagcctacgctgctggcgaactgaaacacggtccgctggcgctaattgatgccgatatgccggttattgttgttgcaccgaacaacgaattgctggaaaaactgaaatccaacattgaagaagttcgcgcgcgtggcggtcagttgtatgtcttcgccgatcaggatgcgggttttgtaagtagcgataacatgcacatcatcgagatgccgcatgtggaagaggtgattgcaccgatcttctacaccgttccgctgcagctgctggcttaccatgtcgcgctgatcaaaggcaccgacgttgaccagccgcgtaacctggcaaaatcggttacggttgagtaa

SEQ ID NO: 58 - DNA, 479 bp, Saccharomyces cerevisiae
tgagcttacccgatggattttatataaggcgaatggaagagggggatttggaacaggtcactgaaacgctaaaggttttgaccaccgtgggcactattacccccgaatccttcagcaaactcataaaatactggaatgaagccacagtatggaatgataacgaagataaaaaaataatgcaatataaccccatggtgattgtggacaagcgcaccgaaacggttgccgctacggggaatatcatcatcgaaagaaagatcattcatgaactggggctatgtggccacatcgaggacattgcagtaaactccaagtatcagggccaaggtttgggcaagctcttgattgatcaattggtaactatcggctttgactacggttgttataagattattttagattgcgatgagaaaaatgtcaaattctatgaaaaatgtgggtttagcaacgcaggcgtggaaatgcaaattagaaaatag

SEQ ID NO: 59 - DNA, 1266 bp, Bacteroides ovatus
atggacagcaaaaacaatattggccatagcgcagatattagcctgaccgcagaactgccgattccgatttataacggtaataccatcatggatttcaaaaaactggccagcctgtataaagatgaactgctggataatgttctgccgttttggctggaacatagccaggatcatgaatatggtggttatttcacctgtctggatcgtgaaggtaaagtgtttgataccgataaattcatttggctgcagagccgtgaagtttggatgtttagcatgctgtataacaaagtcgaaaaacgccaagaatggctggattgtgcaattcagggtggtgaatttctgaaaaaatacggtcatgatggcaactataactggtattttagcctggatcgtagcggtcgtccgctggttgaaccgtataacatttttagctatacctttgccacaatggcatttggtcagctgagcctgaccaccggtaatcaagaatatgcagatattgccaaaaaaaccttcgacatcattctgagcaaagtggataatccgaaaggtcgttggaataaactgcatccgggtacacgtaatctgaaaaactttgcactgccgatgattctgtgtaatctggcactggaaattgaacatctgctggatgaaacctatctgcgtgaaacaatggatacctgcattcatgaagtgatggaagttttttatcgtccggaactgggtggtattattgtggaaaatgttgatattgacggcaacctggttgattgttttgaaggtcgtcaggttacaccgggtcatgccattgaagcaatgtggtttattatggatctgggcaaacgtctgaatcgccctgaactgattgaaaaagcaaaagaaaccacactgaccatgctgaattatggttgggataaacagtatggtggcatctactattttatggatcgtaatggttgtccgcctcagcagctggaatgggatcagaaactgtggtgggttcatattgaaaccctgattagcctgctgaaaggttatcagctgacaggtgacaaaaaatgcctggaatggtttgaaaaagtgcacgattatacctgggagcattttaaagataaagaatatccggaatggtatggctatctgaatcgtcgtggtgaagtgctgctgccgctgaaaggcggtaaatggaaaggttgttttcatgttccgcgtggtctgtatcagtgttggaaaaccctggaagaaatcaaaaacatcgtgagctaa

SEQ ID NO: 60 - DNA, 1044 bp, Campylobacter jejuni
atgaaagagattaagatccagaatattatcatcagcgaagagaaagcgccgctggtggtgccggaaattggcattaaccacaacggcagcctggaactggctaagattatggtggatgccgcgttcagcgccggtgcgaagatcattaaacatcagacgcatatcgttgaagatgagatgagcaaggcggcgaagaaggtgattcctggcaacgccaagattagcatctatgagatcatgcagaaatgcgcgcttgattataaagatgaactggcgctgaaagaatataccgagaagttaggtctggtctatctgtcgacgccattctcgcgcgcaggtgccaaccgtctggaagatatgggcgtgtctgccttcaagattggttccggtgaatgtaataattatccactgatcaagcatattgccgcattcaagaagccgatgattgtcagcaccggcatgaacagcattgaatctatcaaaccgaccgttaagattctgctggataatgagattccgttcgttctgatgcacaccaccaatctgtatccgacgccgcataacctggttcgcctgaacgcgatgctggagctgaagaaggagttctcctgtatggttggcctgagcgatcataccaccgataacctcgcctgtcttggcgcggtggttctcggcgcatgcgtgcttgaacgtcacttcaccgacagcatgcatcgcagcggtccggatatcgtctgctcgatggataccaaggcactgaaggaactgattattcagagcgagcagatggcgattattcgcggcaataacgaatccaagaaggccgccaagcaggaacaggtgaccatcgacttcgcgttcgcttcggtggtcagtattaaggacatcaagaaaggcgaagtgctgtcaatggacaacatctgggtgaagcgtccaggcttaggcggcatcagtgcggcagaattcgagaacattctcggtaagaaggctctgcgcgatattgagaatgatgcgcagctgagctatgaagacttcgcctgataa

SEQ ID NO: 61 - DNA, 666 bp, Campylobacter jejuni
atgtccctggcaatcatccccgctcgcgggggttccaaaggaatcaaaaacaagaacttggtattactgaacaacaagccgctgatttactatacgattaaggctgcattaaatgccaagtctatctctaaggttgtggtttcatccgactccgacgagattttgaactacgctaaaagccaaaatgtagatatcctgaagcgtcccatttctttggcccaggacgacacgacttctgataaagtcttactgcacgcacttaaattttacaaggattatgaagatgtggtatttcttcagcctacatcaccactgcgcaccaacattcacatcaatgaagccttcaatttatacaagaactcgaacgccaacgccttaattagcgtatccgagtgcgacaataagattctgaaggcgtttgtttgtaatgattgcggggatcttgccggtatctgcaatgacgaatacccttttatgccacgccagaaattgccaaagacctacatgtcaaatggagcaatctacatcttgaagattaaggaatttttgaacaacccaagttttttacaatccaagactaagcactttctgatggacgagtcttcatcgcttgacattgactgcctggaagatttgaaaaaggtggagcaaatttggaaaaagtga